(19) United States
(12) Patent Application Publication
(10) Pub. No.: US 2026/0345719 A1
(43) Pub. Date: Nov. 5, 2026

(54) COMPOSITIONS OF A DIPEPTIDYL PEPTIDASE-IV INHIBITOR AND AN ANTIOXIDANT

(71) Applicant: Merck Sharp & Dohme LLC, Rahway, NJ (US)

(72) Inventors: MARCO EUCLIDE ARMENANTE, SOMERSET, NJ (US); MATTHEW KYLE BURRIS, JERSEY CITY, NJ (US); ROBERT CLARK, YOUNGSVILLE, NC (US); MICHAEL B. GENTZLER, WAYNE, PA (US); ANDREW J. GMITTER, COLLEGEVILLE, PA (US); EVANGELIA SPARTALIS, ROSELAND, NJ (US); JINJIAN ZHENG, SCOTCH PLAINS, NJ (US)

(73) Assignee: Merck Sharp & Dohme LLC, Rahway, NJ (US)

(21) Appl. No.: 19/121,464

(22) PCT Filed: Oct. 19, 2023
(86) PCT No.: PCT/US2023/035481

§ 371 Date: Apr. 16, 2025

Related U.S. Application Data
(60) Provisional application No. 63/418,282, filed on Oct. 21, 2022.

(51) Int. Cl. A61K 38/05; A61K 9/28; A61K 31/085; A61K 31/235; A61K 47/02; A61K 47/24; A61K 47/38

(52) U.S. Cl. CPC A61K 38/05; A61K 9/2813; A61K 31/085; A61K 31/235; A61K 47/02; A61K 47/24; A61K 47/38

(57) ABSTRACT

Disclosed are compositions, including pharmaceutical compositions, comprising a combination of sitagliptin, or a pharmaceutically acceptable salt and/or hydrate thereof, and an antioxidant.

### BACKGROUND

Type 2 diabetes is a chronic and progressive disease arising from a complex pathophysiology involving the dual endocrine defects of insulin resistance and impaired insulin secretion. The treatment of Type 2 diabetes typically begins with diet and exercise, followed by oral or injectable anti-diabetic monotherapy. Many patients with type 2 diabetes are considered to be at high risk for coronary artery disease and associated co-morbidities. To reduce the risk of coronary artery disease, it is crucial to manage the entire risk spectrum.

Treatment with cholesterol synthesis inhibitors in patients with and without coronary heart disease, including coronary artery disease, reduces the risk of cardiovascular morbidity and mortality.

Dipeptidyl peptidase-IV (DPP-4) inhibitors represent a class of agents that are being developed for the treatment or improvement in glycemic control in patients with Type 2 diabetes. Specific DPP-4 inhibitors either already approved for marketing or under clinical development for the treatment of Type 2 diabetes include sitagliptin, vildagliptin, saxagliptin, melogliptin, alogliptin denagliptin, carmegliptin linagliptin, dutogliptin. P93/01 (Prosidion), Roche 0730699, TSO21 (Taisho), and E3024 (Eisai). For example, oral administration of sitagliptin, vildagliptin, alogliptin, and saxagliptin to human Type 2 diabetics has been found to reduce fasting glucose and postprandial glucose excursion in association with significantly reduced HbA_{1c} levels. For reviews on the application of DPP-4 inhibitors for the treatment of Type 2 diabetes, reference is made to the following publications: (1) A. H. Stonehouse. et al., “Management of Type 2 diabetes: the role of incretin mimetics, Exp. Opin. Pharmacother., 7: 2095-2105 (2006); (2) B. D. Green, et al., “Inhibition of dipeptidyl peptidase-IV activity as a therapy of Type 2 diabetes,” Exp. Opin. Emerging Drugs, 11: 525-539 (2006); (3) M. M. J. Combettes, “GLP-1 and Type 2 diabetes: physiology and new clinical advances,” Curr. Opin. Pharmacol., 6: 598-605 (2006); and R. K. Campbell. “Rationale for Dipeptidyl Peptidase 4 Inhibitors: A New Class of Oral Agents for the Treatment of Type 2 Diabetes Mellitus,” Ann. Pharmacother., 41: 51-60 (2007).

Sitagliptin phosphate having structural formula I below is the dihydrogenphosphate salt of (2R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine.

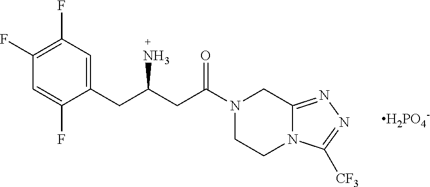

In one embodiment sitagliptin phosphate is in the form of a crystalline monohydrate. Sitagliptin free base and pharmaceutically acceptable salts thereof are disclosed in U.S. Pat. No. 6,699,871 the contents of which are hereby incorporated by reference in their entirety. Crystalline sitagliptin phosphate monohydrate is disclosed in U.S. Pat. No. 7,326,708, the contents of which are hereby incorporated by reference in their entirety. Sitagliptin phosphate has been approved for marketing in several countries, including the U.S., Europe, Canada, and Mexico, for the treatment of Type 2 diabetes and is branded as JANUVIA® as a monotherapy product, and as JANUMET® for the combination therapy product with metformin hydrochloride. For reviews, see D. Drucker, et al., “Sitagliptin.” Nature Reviews Drug Discovery. 6: 109-110 (2007): C. F. Deacon. “Dipeptidyl peptidase 4 inhibition with sitagliptin: a new therapy for Type 2 diabetes,” Exp. Opin. Invest. Drugs, 16: 533-545 (2007); K. A. Lyseng-Williamson, “Sitagliptin,” Drugs, 67: 587-597 (2007); and B. Gallwitz, “Sitagliptin: Profile of a Novel DPP-4 Inhibitor for the Treatment of Type 2 Diabetes (Update),” Drugs of Today, 43: 801-814 (2007).

N-Nitrosamines exist in low levels in water and foods, such as dairy products, meat, and vegetables. In foods, nitrosamine formation and suppression chemistry is typically due to the high temperatures used during cooking and smoking (N. P. Sen et al., “Inhibition of Nitrosamine Formation in Fried Bacon by Propyl Gallate and L-Ascorbyl Palmitate”, J. Agric. Food Chem., 24 (1976), 397: T. Rudlof et al., “Potential Nitrite Scavengers as Inhibitors of the Formation of N-Nitrosamines in Solution and Tobacco Matrix Systems”, J. Agric. Food Chen., 48 (2000), 4381). A preliminary control target of 37 ng/day exposure has been set by major health regulatory authorities.

Antioxidants are used to decrease oxidation, and the resulting oxidative degradation, of active pharmaceutical ingredients in pharmaceutical compositions and formulations.

The use of antioxidants to inhibit nitrosamine formation is discussed in the following: M. Homsak et al, “Assessment of a Diverse Array of Nitrite Scavengers in Solution and Solid State: A Study of Inhibitory Effect on the Formation of Alkyl-Aryl and Dialkyl N-Nitrosamine Derivatives”, Processes, 10 (2022), 2428; S. Pandit, V. H. Grassian, “Gas-Phase Nitrous Acid (HONO) Is Controlled by Surface Interactions of Adsorbed Nitrite (NO₂⁻) on Common Indoor Material Surfaces”, Environ. Sci. Technol., 56 (2022), 12045; I. W. Ashworth et al, “Potential for the Formation of N-Nitrosamines during the Manufacture of Active Pharmaceutical Ingredients: An Assessment of the Risk Posed by Trace Nitrite in Water”, Org. Process Res. Dev., 24 (2020), 1629.

Pharmaceutical compositions comprising combinations of sitagliptin dihydrogen phosphate and metformin hydrochloride are disclosed in WO 2007/078726, WO 2009/099734, and WO 2009/111200.

### SUMMARY

Provided are sitagliptin-antioxidant compositions for use in pharmaceutical compositions of sitagliptin.

Additionally provided are processes to prepare the sitagliptin-antioxidant compositions.

Processes to prepare the sitagliptin-antioxidant composition for use in pharmaceutical compositions and formulations of sitagliptin, or a salt and/or hydrate thereof, are also provided.

The present disclosure provides for sitagliptin-antioxidant compositions that reduce the amount of NTTP formed in formulations and pharmaceutical compositions comprising sitagliptin, or a salt and/or hydrate thereof.

Also provided are pharmaceutical compositions and formulations comprising sitagliptin-antioxidant composition alone or in combination with ertugliflozin, or a salt or co-crystal thereof. These pharmaceutical compositions and formulations may comprise additional excipients.

### BRIEF DESCRIPTION OF THE FIGURES

FIG. 1 is a graph showing NTTP reduction in JANUVIA® core tablets containing either sitagliptin dihydrogen phosphate monohydrate-propyl gallate complex powder (PG) or sitagliptin dihydrogen phosphate monohydrate-butylated hydroxyanisole complex powder (BHA) relative to JANUVIA® core tablets containing sitagliptin dihydrogen phosphate monohydrate powder without an antioxidant (control).

### DETAILED DESCRIPTION

N-Nitrosamines can also sometimes form in drug formulations via chemical reactions between nitrites and amines in the formulation during manufacturing formulation and storage.

It was unexpectedly found that there is a potential to form nitrosamines, in particular N-7-nitroso-3-(trifluoromethyl)-5,6,7,8-tetrahydro[1,2,4]triazolo-[4,3-a]pyrazine (NTTP), in sitagliptin formulations. NTTP may be formed due to nitrosation of 3-(trifluoromethyl)-5,6,7,8-tetrahydro-[1,2,4]triazolo-[4,3-a]pyrazine, formed by hydrolysis of sitagliptin, by trace nitrite species in the formulation, such as nitrous acid nitrosonium ions, nitric oxide and mixed nitrite salts.

There is a need for a sitagliptin formulation that reduces the amount of the N-Nitrosamine 7-nitroso-3-(trifluoromethyl)-5,6,7,8-tetrahydro-[1,2,4]triazolo[4,3-a]pyrazine (also known as NTTP) formed during manufacturing, formulation and storage.

It has been unexpectedly found that some antioxidants also work to reduce the formation and growth of N-nitrosamines, including NTTP, in the presence of nitrites in solution and in formulations or pharmaceutical compositions containing sitagliptin, or salts and/o rhydrates thereof. Without being limited to any particular theory it is proposed that the antioxidants may work by reacting with nitrite and with NO+, the nitrosating agent formed from nitrite, in these formulations and pharmaceutical compositions. It has been further unexpectedly found that antioxidants, including but not limited to butylated hydroxyanisole (BHA) and propyl gallate (PG), can react with nitrites to prevent the formation of NTTP in sitagliptin dihydrogen phosphate monohydrate solutions, formulations, and pharmaceutical compositions.

The present disclosure relates to antioxidant-containing compositions, pharmaceutical compositions, and formulations of sitagliptin and salts and/or hydrates thereof, that minimize or reduce NTTP formation. In particular, the present disclosure relates to antioxidant-containing compositions, pharmaceutical compositions and formulations of sitagliptin dihydrogen phosphate monohydrate that minimize or reduce NTTP formation. In one embodiment, the pharmaceutical compositions and formulations may be used in compressed tablets for oral administration. Additionally, the antioxidant containing compositions, pharmaceutical compositions and formulations of the present disclosure inhibit or reduce NTTP formation without increasing sitagliptin chemical degradation.

The present disclosure also relates to antioxidant-containing compositions, pharmaceutical compositions and formulations of sitagliptin, and salts and/or hydrates thereof in a fixed dose combination with ertugliflozin, or a salt or co-crystal thereof, that reduce NTTP formation. In particular, the present disclosure relates to antioxidant-containing compositions, pharmaceutical compositions and formulations of sitagliptin dihydrogen phosphate monohydrate in a fixed dose combination with ertugliflozin L-pyroglutamic acid co-crystal, that reduce NTTP formation. In one embodiment, the pharmaceutical compositions and formulations may be used in compressed tablets for oral administration. Additionally, the antioxidant containing compositions, pharmaceutical compositions, and formulations of the present disclosure inhibit or reduce NTTP formation without increasing sitagliptin chemical degradation.

It has been surprisingly found that compositions, pharmaceutical compositions formulations and tablets containing sitagliptin dihydrogen phosphate monohydrate and an antioxidant resulted in the inhibition of NTTP formation over time relative to compositions and pharmaceutical compositions, formulations and tablets without an antioxidant over an equivalent period of time. It has been further found that the use of antioxidant in sitagliptin compositions, pharmaceutical compositions, formulations and tablets reduces NTTP formation without increasing sitagliptin chemical degradation.

It has also surprisingly been found that compositions, pharmaceutical compositions, formulations and tablets containing sitagliptin dihydrogen phosphate monohydrate in combination with ertugliflozin L-pyroglutamic acid co-crystal, and an antioxidant resulted in the inhibition of NTTP formation over a period of time relative to compositions and pharmaceutical compositions, formulations and tablets without an antioxidant over an equivalent period of time. It was also found that the use of antioxidant in sitagliptin and ertugliflozin compositions, pharmaceutical compositions, formulations and tablets reduces NTTP formation without increasing sitagliptin chemical degradation.

It has also been unexpectedly found that the antioxidants are effective when added to compositions, pharmaceutical compositions and formulations of sitagliptin dihydrogen phosphate monohydrate, prior to tableting, through multiple routes, including: 1) formation of a sitagliptin antioxidant composition, including but not limited to, a sitagliptin dihydrogen phosphate monohydrate antioxidant composition, and addition of the sitagliptin antioxidant composition to the dry pharmaceutical composition ingredients prior to tablet formation; 2) addition of the antioxidant as a dry powder to the pharmaceutical composition of sitagliptin dihydrogen phosphate monohydrate prior to tablet formation; and 3) deposition of the antioxidant on an excipient and addition of the antioxidant excipient composition to the pharmaceutical composition of sitagliptin dihydrogen phosphate monohydrate prior to tablet formation.

Additionally, it was unexpectedly found that compositions, pharmaceutical compositions, formulations and tablets containing sitagliptin dihydrogen phosphate monohydrate, metformin and an antioxidant did not consistently result in the inhibition of NTTP formation over time relative to compositions and pharmaceutical compositions, formulations and tablets without an antioxidant over an equivalent period of time.

One aspect of the present disclosure is directed to a composition comprising sitagliptin, or a pharmaceutically acceptable salt and/or hydrate thereof, and an antioxidant.

The present disclosure provides for a pharmaceutical composition comprising a sitagliptin phosphate-antioxidant composition. In one embodiment, sitagliptin phosphate-antioxidant composition comprises sitagliptin dihydrogen phosphate and an antioxidant. In another embodiment, sitagliptin phosphate-antioxidant composition comprises sitagliptin dihydrogen phosphate monohydrate and an antioxidant. In another embodiment, the sitagliptin dihydrogen phosphate monohydrate and antioxidant composition is a solid. In another embodiment, the sitagliptin dihydrogen phosphate monohydrate and antioxidant composition is a crystalline solid.

In another embodiment, sitagliptin phosphate-antioxidant composition comprises sitagliptin dihydrogen phosphate and propyl gallate. In another embodiment, sitagliptin phosphate-antioxidant composition comprises sitagliptin dihydrogen phosphate monohydrate and propyl gallate. In another embodiment, the sitagliptin dihydrogen phosphate monohydrate and propyl gallate composition is a solid. In another embodiment, the sitagliptin dihydrogen phosphate monohydrate and propyl gallate composition is a crystalline solid.

In another embodiment, sitagliptin phosphate-antioxidant composition comprises sitagliptin dihydrogen phosphate and butylated hydroxyanisole. In another embodiment sitagliptin phosphate-antioxidant composition comprises sitagliptin dihydrogen phosphate monohydrate and butylated hydroxyanisole. In another embodiment, the sitagliptin dihydrogen phosphate monohydrate and butylated hydroxyanisole composition is a solid. In another embodiment, the sitagliptin dihydrogen phosphate monohydrate and butylated hydroxyanisole composition is a crystalline solid.

In another embodiment, the sitagliptin dihydrogen phosphate monohydrate-antioxidant composition is used in a pharmaceutical composition.

The present disclosure further provides for pharmaceutical compositions comprising sitagliptin dihydrogen phosphate monohydrate-antioxidant compositions for use in pharmaceutical compositions, formulations and tablets.

The present disclosure includes pharmaceutical compositions of sitagliptin, or a pharmaceutically acceptable salt thereof, and an antioxidant. Also included are pharmaceutical compositions of sitagliptin dihydrogen phosphate monohydrate and an antioxidant. The pharmaceutical compositions may include additional excipients, including but not limited to, microcrystalline cellulose, dicalcium phosphate, croscarmellose sodium, magnesium stearate, and/or sodium stearyl fumarate. The pharmaceutical compositions may be compressed into tablets and optionally film coated.

In one embodiment, the pharmaceutical composition contains 32.13 mg, 64.25 mg, or 128.5 mg of sitagliptin dihydrogen phosphate monohydrate, which is equivalent to 25 mg, 50 mg, or 100 mg, respectively, of the sitagliptin free base. In another embodiment, the pharmaceutical composition comprises one or more of the following ingredients: microcrystalline cellulose, anhydrous dibasic calcium phosphate, croscarmellose sodium, magnesium stearate, and sodium stearyl fumarate. In another embodiment, the pharmaceutical composition comprises: microcrystalline cellulose, anhydrous dibasic calcium phosphate, croscarmellose sodium, magnesium stearate, and sodium stearyl fumarate. In another embodiment, the pharmaceutical composition may be formed into a tablet. In another embodiment, the tablet may be covered with a film coating. In another embodiment, the film coating comprises: polyvinyl alcohol, polyethylene glycol, talc, titanium dioxide, red iron oxide, and yellow iron oxide.

In one embodiment, the pharmaceutical composition comprises:
a) 10-50 weight % sitagliptin dihydrogen phosphate monohydrate;
b) 0.001-1 weight % antioxidant:
c) 10-50 weight % microcrystalline cellulose;
d) 10-50 weight % anhydrous dibasic calcium phosphate;
e) 0.5-5 weight % croscarmellose sodium;
f) 0.5-5% weight % sodium stearyl fumarate; and
g) 0.5-2 weight % magnesium stearate.

In a class of this embodiment, the antioxidant is butylated hydroxyanisole. In a subclass of this class, the particle size of butylated hydroxyanisole is ≤400 microns. In another subclass of this class, the particle size of butylated hydroxyanisole is 1 to 400 microns. In another subclass of this class, the particle size of butylated hydroxyanisole is ≤375 microns. In another subclass of this class, the particle size of butylated hydroxyanisole is 1 to 375 microns. In another subclass of this class, the particle size of butylated hydroxyanisole is 22 to 375 microns. In another subclass of this class, the particle size of butylated hydroxanisole is ≤150 microns. In another subclass of this class, the particle size of butylated hydroxyanisole is 1 to 150 microns. In another subclass of this class, the particle size of butylated hydroxyanisole is ≤83 microns. In another subclass of this class, the particle size of butylated hydroxyanisole is 1 to 83 microns. In another subclass of this class, the particle size of butylated hydroxyanisole is ≤44 microns. In another subclass of this class, the particle size of butylated hydroxyanisole is 1 to 44 microns. In another subclass of this class, the particle size of butylated hydroxyanisole is ≤22 microns. In another subclass of this class, the particle size of butylated hydroxyanisole is 1 to 22 microns.

In another class of this embodiment, the antioxidant is propyl gallate. In a subclass of this class, the particle size of propyl gallate is ≤400 microns. In another subclass of this class, the particle size of propyl gallate is 1 to 400 microns. In another subclass of this class, the particle size of propyl gallate is ≤375 microns. In another subclass of this class, the particle size of propyl gallate is 1 to 375 microns. In another subclass of this class, the particle size of propyl gallate is 22 to 375 microns. In another subclass of this class, the particle size of propyl gallate is ≤150 microns. In another subclass of this class, the particle size of propyl gallate is 1 to 150 microns. In another subclass of this class, the particle size of propyl gallate is ≤83 microns. In another subclass of this class, the particle size of propyl gallate is 1 to 83 microns.

In another subclass of this class, the particle size of propyl gallate is ≤44 microns. In another subclass of this class, the particle size of propyl gallate is 1 to 44 microns. In another subclass of this class, the particle size of propyl gallate is ≤22 microns. In another subclass of this class, the particle size of propyl gallate is 1 to 22 microns.

In another class of this embodiment, the pharmaceutical composition is formed into a tablet. In a subclass of this class, the tablet is film coated. In another subclass, the tablet is film coated with Opadry 85F. In another subclass, the tablet is film coated with 2-8 weight % of Opadry 85F.

In one embodiment, the pharmaceutical composition comprises:
a) 21-50 weight % sitagliptin dihydrogen phosphate monohydrate;
b) 0.001-1 weight % antioxidant;
c) 10-50 weight % microcrystalline cellulose;
d) 10-50 weight % anhydrous dibasic calcium phosphate;
e) 0.5-5 weight % croscarmellose sodium;
f) 0.5-5% weight % sodium stearyl fumarate; and
g) 0.5-2 weight % magnesium stearate.

In a class of this embodiment, the antioxidant is butylated hydroxyanisole. In a subclass of this class, the particle size of butylated hydroxyanisole is ≤400 microns. In another subclass of this class, the particle size of butylated hydroxyanisole is 1 to 400 microns. In another subclass of this class, the particle size of butylated hydroxyanisole is ≤375 microns. In another subclass of this class, the particle size of butylated hydroxyanisole is 1 to 375 microns. In another subclass of this class, the particle size of butylated hydroxyanisole is 22 to 375 microns. In another subclass of this class, the particle size of butylated hydroxanisole is ≤150 microns. In another subclass of this class, the particle size of butylated hydroxyanisole is 1 to 150 microns. In another subclass of this class, the particle size of butylated hydroxyanisole is ≤83 microns. In another subclass of this class, the particle size of butylated hydroxyanisole is 1 to 83 microns. In another subclass of this class, the particle size of butylated hydroxyanisole is ≤44 microns. In another subclass of this class, the particle size of butylated hydroxyanisole is 1 to 44 microns. In another subclass of this class, the particle size of butylated hydroxyanisole is ≤22 microns. In another subclass of this class, the particle size of butylated hydroxyanisole is 1 to 22 microns.

In another class of this embodiment, the antioxidant is propyl gallate. In a subclass of this class, the particle size of propyl gallate is ≤400 microns. In another subclass of this class, the particle size of propyl gallate is 1 to 400 microns. In another subclass of this class, the particle size of propyl gallate is ≤375 microns. In another subclass of this class, the particle size of propyl gallate is 1 to 375 microns. In another subclass of this class, the particle size of propyl gallate is 22 to 375 microns. In another subclass of this class, the particle size of propyl gallate is ≤150 microns. In another subclass of this class, the particle size of propyl gallate is 1 to 150 microns. In another subclass of this class, the particle size of propyl gallate is ≤83 microns. In another subclass of this class, the particle size of propyl gallate is 1 to 83 microns.

In another subclass of this class, the particle size of propyl gallate is ≤44 microns. In another subclass of this class, the particle size of propyl gallate is 1 to 44 microns. In another subclass of this class, the particle size of propyl gallate is ≤22 microns. In another subclass of this class, the particle size of propyl gallate is 1 to 22 microns.

In another class of this embodiment, the pharmaceutical composition is formed into a tablet. In a subclass of this class, the tablet is film coated. In another subclass, the tablet is film coated with Opadry 85F. In another subclass, the tablet is film coated with 2-8 weight % of Opadry 85F.

In another embodiment, the pharmaceutical composition comprises:
a) 10-50 weight % sitagliptin dihydrogen phosphate monohydrate;
b) 0.001-1 weight % antioxidant;
c) 10-50 weight % microcrystalline cellulose;
d) 10-50 weight % anhydrous dibasic calcium phosphate:
e) 0.5-5 weight % croscarmellose sodium;
f) 0.5-5 weight % sodium stearyl fumarate; and
g) 0-2 weight % magnesium stearate.

In a class of this embodiment, the antioxidant is butylated hydroxyanisole. In a subclass of this class, the particle size of butylated hydroxyanisole is ≤400 microns. In another subclass of this class, the particle size of butylated hydroxyanisole is 1 to 400 microns.

In another subclass of this class, the particle size of butylated hydroxyanisole is ≤375 microns.

In another subclass of this class, the particle size of butylated hydroxyanisole is 1 to 375 microns. In another subclass of this class, the particle size of butylated hydroxyanisole is 22 to 375 microns. In another subclass of this class, the particle size of butylated hydroxyanisole is ≤150 microns. In another subclass of this class, the particle size of butylated hydroxyanisole is 1 to 150 microns. In another subclass of this class, the particle size of butylated hydroxyanisole is ≤83 microns. In another subclass of this class the particle size of butylated hydroxyanisole is 1 to 83 microns. In another subclass of this class, the particle size of butylated hydroxyanisole is ≤44 microns. In another subclass of this class, the particle size of butylated hydroxyanisole is 1 to 44 microns. In another subclass of this class, the particle size of butylated hydroxyanisole is ≤22 microns. In another subclass of this class, the particle size of butylated hydroxyanisole is 1 to 22 microns.

In another class of this embodiment, the antioxidant is propyl gallate. In a subclass of this class, the particle size of propyl gallate is ≤400 microns. In another subclass of this class the particle size of propyl gallate is 1 to 400 microns. In another subclass of this class the particle size of propyl gallate is ≤375 microns. In another subclass of this class, the particle size of propyl gallate is 1 to 375 microns. In another subclass of this class, the particle size of propyl gallate is 22 to 375 microns. In another subclass of this class, the particle size of propyl gallate is ≤150 microns. In another subclass of this class, the particle size of propyl gallate is 1 to 150 microns. In another subclass of this class, the particle size of propyl gallate is ≤83 microns. In another subclass of this class, the particle size of propyl gallate is 1 to 83 microns.

In another subclass of this class, the particle size of propyl gallate is ≤44 microns. In another subclass of this class, the particle size of propyl gallate is 1 to 44 microns. In another subclass of this class, the particle size of propyl gallate is ≤22 microns. In another subclass of this class, the particle size of propyl gallate is 1 to 22 microns.

In another class of this embodiment, the pharmaceutical composition is formed into a tablet. In a subclass of this class, the tablet is film coated. In another subclass, the tablet is film coated with Opadry 85F. In another subclass, the tablet is film coated with 2-8 weight % of Opadry 85F.

In another embodiment, the pharmaceutical composition comprises:
a) 21-50 weight % sitagliptin dihydrogen phosphate monohydrate;
b) 0.001-1 weight % antioxidant;
c) 10-50 weight % microcrystalline cellulose;
d) 10-50 weight % anhydrous dibasic calcium phosphate;
e) 0.5-5 weight % croscarmellose sodium:
f) 0.5-5 weight % sodium stearyl fumarate; and
g) 0-2 weight % magnesium stearate.

In a class of this embodiment, the antioxidant is butylated hydroxyanisole. In a subclass of this class, the particle size of butylated hydroxyanisole is ≤400 microns. In another subclass of this class, the particle size of butylated hydroxyanisole is 1 to 400 microns. In another subclass of this class, the particle size of butylated hydroxyanisole is ≤375 microns.

In another subclass of this class, the particle size of butylated hydroxyanisole is 1 to 375 microns. In another subclass of this class, the particle size of butylated hydroxyanisole is 22 to 375 microns. In another subclass of this class, the particle size of butylated hydroxyanisole is ≤150 microns. In another subclass of this class, the particle size of butylated hydroxyanisole is 1 to 150 microns. In another subclass of this class, the particle size of butylated hydroxyanisole is ≤83 microns. In another subclass of this class, the particle size of butylated hydroxyanisole is 1 to 83 microns. In another subclass of this class, the particle size of butylated hydroxyanisole is ≤44 microns. In another subclass of this class, the particle size of butylated hydroxyanisole is 1 to 44 microns. In another subclass of this class, the particle size of butylated hydroxyanisole is ≤22 microns. In another subclass of this class, the particle size of butylated hydroxyanisole is 1 to 22 microns.

In another class of this embodiment, the antioxidant is propyl gallate. In a subclass of this class, the particle size of propyl gallate is ≤400 microns. In another subclass of this class, the particle size of propyl gallate is 1 to 400 microns. In another subclass of this class, the particle size of propyl gallate is ≤375 microns. In another subclass of this class, the particle size of propyl gallate is 1 to 375 microns. In another subclass of this class, the particle size of propyl gallate is 22 to 375 microns. In another subclass of this class, the particle size of propyl gallate is ≤150 microns. In another subclass of this class, the particle size of propyl gallate is 1 to 150 microns. In another subclass of this class, the particle size of propyl gallate is ≤83 microns. In another subclass of this class, the particle size of propyl gallate is 1 to 83 microns.

In another subclass of this class, the particle size of propyl gallate is ≤44 microns. In another subclass of this class, the particle size of propyl gallate is 1 to 44 microns. In another subclass of this class, the particle size of propyl gallate is ≤22 microns. In another subclass of this class, the particle size of propyl gallate is 1 to 22 microns.

In another class of this embodiment, the pharmaceutical composition is formed into a tablet. In a subclass of this class, the tablet is film coated. In another subclass, the tablet is film coated with Opadry 85F. In another subclass, the tablet is film coated with 2-8 weight % of Opadry 85F.

In another embodiment, the sitagliptin dihydrogen phosphate monohydrate and antioxidant pharmaceutical composition is prepared by formation of a sitagliptin dihydrogen phosphate monohydrate-antioxidant composition and addition of the composition or complex to the dry pharmaceutical composition ingredients prior to tablet formation. In a class of this embodiment, the antioxidant is butylated hydroxyanisole. In another class of this embodiment, the antioxidant is propyl gallate.

In another embodiment, the sitagliptin dihydrogen phosphate monohydrate and antioxidant pharmaceutical composition is prepared by addition of the antioxidant as a dry powder to the pharmaceutical composition of sitagliptin dihydrogen phosphate monohydrate prior to tablet formation.

In a class of this embodiment, the antioxidant is butylated hydroxyanisole. In a subclass of this class, the particle size of butylated hydroxyanisole is ≤400 microns. In another subclass of this class, the particle size of butylated hydroxyanisole is 1 to 400 microns. In another subclass of this class, the particle size of butylated hydroxyanisole is ≤375 microns. In another subclass of this class, the particle size of butylated hydroxyanisole is 1 to 375 microns. In another subclass of this class, the particle size of butylated hydroxyanisole is 22 to 375 microns. In another subclass of this class, the particle size of butylated hydroxyanisole is ≤150 microns. In another subclass of this class, the particle size of butylated hydroxyanisole is 1 to 150 microns. In another subclass of this class, the particle size of butylated hydroxyanisole is ≤83 microns. In another subclass of this class, the particle size of butylated hydroxyanisole is 1 to 83 microns. In another subclass of this class, the particle size of butylated hydroxyanisole is ≤44 microns. In another subclass of this class, the particle size of butylated hydroxyanisole is 1 to 44 microns. In another subclass of this class, the particle size of butylated hydroxyanisole is ≤22 microns. In another subclass of this class, the particle size of butylated hydroxyanisole is 1 to 22 microns.

In another class of this embodiment, the antioxidant is propyl gallate. In a subclass of this class, the particle size of propyl gallate is ≤400 microns. In another subclass of this class, the particle size of propyl gallate is 1 to 400 microns. In another subclass of this class, the particle size of propyl gallate is ≤375 microns. In another subclass of this class, the particle size of propyl gallate is 1 to 375 microns. In another subclass of this class, the particle size of propyl gallate is 22 to 375 microns. In another subclass of this class, the particle size of propyl gallate is ≤150 microns. In another subclass of this class, the particle size of propyl gallate is 1 to 150 microns. In another subclass of this class, the particle size of propyl gallate is ≤83 microns. In another subclass of this class, the particle size of propyl gallate is 1 to 83 microns. In another subclass of this class, the particle size of propyl gallate is ≤44 microns. In another subclass of this class, the particle size of propyl gallate is 1 to 44 microns. In another subclass of this class, the particle size of propyl gallate is ≤22 microns. In another subclass of this class, the particle size of propyl gallate is 1 to 22 microns.

In another embodiment, the sitagliptin dihydrogen phosphate monohydrate and antioxidant pharmaceutical composition is prepared by deposition of the antioxidant on an excipient and addition of the antioxidant/excipient composition to the pharmaceutical composition of sitagliptin dihydrogen phosphate monohydrate prior to tablet formation. In a class of this embodiment, the antioxidant is butylated hydroxvanisole. In another class of this embodiment, the antioxidant is propyl gallate.

In another embodiment, the sitagliptin phosphate-antioxidant composition is used in the JANUVIA® pharmaceutical composition. In another embodiment, the sitagliptin dihydrogen phosphate monohydrate-antioxidant composition is used in the pharmaceutical composition for JANUVIA®. In another embodiment, the sitagliptin phosphate-antioxidant composition is used in JANUVIA® tablets. In another embodiment, the sitagliptin dihydrogen phosphate monohydrate-antioxidant composition is used in JANUVIA® tablets. In another embodiment, sitagliptin phosphate-antioxidant composition comprises sitagliptin dihydrogen phosphate and an antioxidant. In another embodiment, sitagliptin phosphate-antioxidant composition comprises sitagliptin dihydrogen phosphate and propyl gallate. In another embodiment, sitagliptin phosphate-antioxidant composition comprises sitagliptin dihydrogen phosphate monohydrate and propyl gallate. In another embodiment, the sitagliptin dihydrogen phosphate monohydrate-propyl gallate composition is used in JANUVIA® tablets. In another embodiment, sitagliptin phosphate-antioxidant composition comprises sitagliptin dihydrogen phosphate and butylated hydroxyanisole. In another embodiment, sitagliptin phosphate-antioxidant composition comprises sitagliptin dihydrogen phosphate monohydrate and butylated hydroxyanisole. In another embodiment, the sitagliptin dihydrogen phosphate monohydrate-butylated hydroxyanisole composition is used in JANUVIA® tablets.

In another embodiment, a core tablet comprising sitagliptin prepared by the addition of the sitagliptin dihydrogen phosphate monohydrate antioxidant composition wherein the NTTP level is less than or equal to 4 ppm. In a class of this embodiment, the core tablet prepared by the addition of the sitagliptin dihydrogen phosphate monohydrate antioxidant composition wherein the NTTP level is less than or equal to 4 ppm after storing at 70 °C for 2.5 days. In another class of this embodiment, the core tablet prepared by the addition of the sitagliptin dihydrogen phosphate monohydrate antioxidant composition wherein the NTTP level is less than or equal to 4 ppm after storing at 60 °C and 60% relative humidity for 1.5 weeks; after storing at 70 °C and 60% relative humidity for 2.5 days; after storing at 40 °C and 75% relative humidity for 1.5 months: or after storing at 40 °C and 65% relative humidity for 1.5 weeks. In another class of this embodiment, the antioxidant is propyl gallate or butylated hydroxyanisole.

In another class of this embodiment, the antioxidant is propyl gallate. In another class of this embodiment, the core tablet is a Januvia® 25 mg core tablet, a Januvia® 50 mg core tablet, or a Januvia® 100 mg core tablet. In another class of this embodiment, the core tablet further comprises a film coating.

In another embodiment, a core tablet comprising sitagliptin prepared by the addition of the sitagliptin dihydrogen phosphate monohydrate antioxidant composition wherein the NTTP level is less than or equal to 2 ppm. In a class of this embodiment, the core tablet prepared by the addition of the sitagliptin dihydrogen phosphate monohydrate antioxidant composition wherein the NTTP level is less than or equal to 2 ppm after storing at 70 °C for 2.5 days. In another class of this embodiment, the core tablet prepared by the addition of the sitagliptin dihydrogen phosphate monohydrate antioxidant composition wherein the NTTP level is less than or equal to 2 ppm after storing at 60 °C and 60% relative humidity for 1.5 weeks; after storing at 70 °C and 60% relative humidity for 2.5 days; after storing at 40 °C and 75% relative humidity for 1.5 months: or after storing at 40 °C and 65% relative humidity for 1.5 weeks. In another class of this embodiment, the antioxidant is propyl gallate or butylated hydroxyanisole. In another class of this embodiment, the antioxidant is propyl gallate. In another class of this embodiment, the core tablet is a Januvia® 25 mg_core tablet, a Januvia® 50 mg core tablet, or a Januvia® 100 mg core tablet. In another class of this embodiment, the core tablet further comprises a film coating.

In another embodiment, a core tablet comprising sitagliptin prepared by the addition of the sitagliptin dihydrogen phosphate monohydrate antioxidant composition wherein the NTTP level is less than or equal to 1 ppm. In a class of this embodiment, the core tablet prepared by the addition of the sitagliptin dihydrogen phosphate monohydrate antioxidant composition wherein the NTTP level is less than or equal to 1 ppm after storing at 70 °C for 2.5 days. In another class of this embodiment, the core tablet prepared by the addition of the sitagliptin dihydrogen phosphate monohydrate antioxidant composition wherein the NTTP level is less than or equal to 1 ppm after storing at 60 °C and 60% relative humidity for 1.5 weeks; after storing at 70 °C and 60% relative humidity for 2.5 days: after storing at 40 °C and 75% relative humidity for 1.5 months; or after storing at 40 °C and 65% relative humidity for 1.5 weeks. In another class of this embodiment, the antioxidant is propyl gallate or butylated hydroxyanisole. In another class of this embodiment, the antioxidant is propyl gallate. In another class of this embodiment, the core tablet is a Januvia® 25 mg core tablet, a Januvia® 50 mg core tablet, or a Januvia® 100 mg core tablet. In another class of this embodiment, the core tablet further comprises a film coating.

In another embodiment, a core tablet comprising sitagliptin prepared by the addition of the sitagliptin dihydrogen phosphate monohydrate antioxidant composition wherein the NTTP level is less than or equal to 0.37 ppm. In a class of this embodiment, the core tablet prepared by the addition of the sitagliptin dihydrogen phosphate monohydrate antioxidant composition wherein the NTTP level is less than or equal to 0.37 ppm after storing at 70 °C for 2.5 days. In another class of this embodiment, the core tablet prepared by the addition of the sitagliptin dihydrogen phosphate monohydrate antioxidant composition wherein the NTTP level is less than or equal to 0.37 ppm after storing at 60 °C and 60% relative humidity for 1.5 weeks: after storing at 70 °C and 60% relative humidity for 2.5 days; after storing at 40 °C and 75% relative humidity for 1.5 months; or after storing at 40 °C and 65% relative humidity for 1.5 weeks. In another class of this embodiment, the antioxidant is propyl gallate or butylated hydroxyanisole. In another class of this embodiment, the antioxidant is propyl gallate. In another class of this embodiment, the core tablet is a Januvia® 25 mg core tablet, a Januvia® 50 mg core tablet, or a Januvia® 100 mg core tablet. In another class of this embodiment, the core tablet further comprises a film coating.

In another embodiment, a core tablet comprising sitagliptin prepared by the addition of an antioxidant as a dry powder wherein the NTTP level is less than or equal to 4 ppm. In a class of this embodiment, the core tablet prepared by the addition of an antioxidant as a dry powder wherein the NTTP level is less than or equal to 4 ppm after storing at 70 °C for 2.5 days. In another class of this embodiment, the core tablet prepared by the addition of an antioxidant as a dry powder wherein the NTTP level is less than or equal to 4 ppm after storing at 60 °C and 60% relative humidity for 1.5 weeks; after storing at 70 °C and 60% relative humidity for 2.5 days; after storing at 40 °C and 75% relative humidity for 1.5 months; or after storing at 40 °C and 65% relative humidity for 1.5 weeks. In another class of this embodiment, the antioxidant is propyl gallate or butylated hydroxyanisole. In another class of this embodiment, the antioxidant is propyl gallate. In another class of this embodiment, the core tablet is a Januvia® 25 mg core tablet, a Januvia® 50 mg core tablet, or a Januvia® 100 mg core tablet. In another class of this embodiment, the core tablet further comprises a film coating. In another class of this embodiment, sitagliptin is sitagliptin dihydrogen phosphate. In another class of this embodiment, sitagliptin is sitagliptin dihydrogen phosphate monohydrate.

In another embodiment, a core tablet comprising sitagliptin prepared by the addition of an antioxidant as a dry powder wherein the NTTP level is less than or equal to 2 ppm. In a class of this embodiment, the core tablet prepared by the addition of an antioxidant as a dry powder wherein the NTTP level less than or equal to 2 ppm after storing at 70 °C for 2.5 days. In another class of this embodiment, the core tablet prepared by the addition of an antioxidant as a dry powder wherein the NTTP level is less than or equal to 2 ppm after storing at 60 °C and 60% relative humidity for 1.5 weeks; after storing at 70 °C and 60% relative humidity for 2.5 days; after storing at 40 °C and 75% relative humidity for 1.5 months; or after storing at 40 °C and 65% relative humidity for 1.5 weeks. In another class of this embodiment, the antioxidant is propyl gallate or butylated hydroxyanisole. In another class of this embodiment, the antioxidant is propyl gallate. In another class of this embodiment, the core tablet is a Januvia® 25 mg core tablet, a Januvia® 50 mg core tablet, or a Januvia® k 100 mg core tablet. In another class of this embodiment, the core tablet further comprises a film coating. In another class of this embodiment, sitagliptin is sitagliptin dihydrogen phosphate. In another class of this embodiment, sitagliptin is sitagliptin dihydrogen phosphate monohydrate.

In another embodiment, a core tablet comprising sitagliptin prepared by the addition of an antioxidant as a dry powder wherein the NTTP level is less than or equal to 1 ppm. In a class of this embodiment, the core tablet prepared by the addition of an antioxidant as a dry powder wherein the NTTP level is less than or equal to 1 ppm after storing at 70 °C for 2.5 days. In another class of this embodiment, the core tablet prepared by the addition of an antioxidant as a dry powder wherein the NTTP level is less than or equal to 1 ppm after storing at 60 °C and 60% relative humidity for 1.5 weeks; after storing at 70 °C and 60% relative humidity for 2.5 days; after storing at 40 °C and 75% relative humidity for 1.5 months; or after storing at 40 °C and 65% relative humidity for 1.5 weeks. In another class of this embodiment, the antioxidant is propyl gallate or butylated hydroxyanisole. In another class of this embodiment, the antioxidant is propyl gallate. In another class of this embodiment, the core tablet is a Januvia® 25 mg core tablet, a Januvia® 50 mg core tablet, or a Januvia® 100 mg core tablet. In another class of this embodiment, the core tablet further comprises a film coating. In another class of this embodiment, sitagliptin is sitagliptin dihydrogen phosphate. In another class of this embodiment, sitagliptin is sitagliptin dihydrogen phosphate monohydrate.

In another embodiment, a core tablet comprising sitagliptin prepared by the addition of an antioxidant as a dry powder wherein the NTTP level is less than or equal to 0.37 ppm. In a class of this embodiment, the core tablet prepared by the addition of an antioxidant as a dry powder wherein the NTTP level is less than or equal to 0.37 ppm after storing at 70 °C for 2.5 days. In another class of this embodiment, the core tablet prepared by the addition of an antioxidant as a dry powder wherein the NTTP level is less than or equal to 0.37 ppm after storing at 60 °C and 60% relative humidity for 1.5 weeks; after storing at 70 °C and 60% relative humidity for 2.5 days; after storing at 40 °C and 75% relative humidity for 1.5 months; or after storing at 40 °C and 65% relative humidity for 1.5 weeks. In another class of this embodiment, the antioxidant is propyl gallate or butylated hydroxyanisole. In another class of this embodiment, the antioxidant is propyl gallate. In another class of this embodiment, the core tablet is a Januvia® 25 mg core tablet, a Januvia® 50 mg core tablet, or a Januvia® 100 mg core tablet. In another class of this embodiment, the core tablet further comprises a film coating. In another class of this embodiment, sitagliptin is sitagliptin dihydrogen phosphate. In another class of this embodiment, sitagliptin is sitagliptin dihydrogen phosphate monohydrate.

In another embodiment, a core tablet comprising sitagliptin prepared by the addition of an antioxidant deposited on an excipient wherein the NTTP level is less than or equal to 4 ppm. In a class of this embodiment, the core tablet prepared by the addition of an antioxidant deposited on an excipient wherein the NTTP level is less than or equal to 4 ppm after storing at 70 °C for 2.5 days. In another class of this embodiment, the core tablet prepared by the addition of an antioxidant deposited on an excipient wherein the NTTP level is less than or equal to 4 ppm after storing at 60 °C and 60% relative humidity for 1.5 weeks; after storing at 70 °C and 60% relative humidity for 2.5 days; after storing at 40 °C and 75% relative humidity for 1.5 months; or after storing at 40 °C and 65% relative humidity for 1.5 weeks. In another class of this embodiment, the antioxidant is propyl gallate and the excipient is dicalcium phosphate. In another class of this embodiment, the core tablet is a Januvia® 25 mg core tablet, a Januvia® 50 mg core tablet, or a Januvia® 100 mg core tablet. In another class of this embodiment, the core tablet further comprises a film coating. In another class of this embodiment, sitagliptin is sitagliptin dihydrogen phosphate. In another class of this embodiment, sitagliptin is sitagliptin dihydrogen phosphate monohydrate.

In another embodiment, a core tablet comprising sitagliptin prepared by the addition of an antioxidant deposited on an excipient wherein the NTTP level is less than or equal to 2 ppm.

In a class of this embodiment, the core tablet prepared by the addition of an antioxidant deposited on an excipient wherein the NTTP level is less than or equal to 2 ppm after storing at 70 °C for 2.5 days. In another class of this embodiment, the core tablet prepared by the addition of an antioxidant deposited on an excipient wherein the NTTP level is less than or equal to 2 ppm after storing at 60 °C and 60% relative humidity for 1.5 weeks; after storing at 70 °C and 60% relative humidity for 2.5 days; after storing at 40 °C and 75% relative humidity for 1.5 months; or after storing at 40 °C and 65% relative humidity for 1.5 weeks. In another class of this embodiment, the antioxidant is propyl gallate and the excipient is dicalcium phosphate. In another class of this embodiment, the core tablet is a Januvia® 25 mg core tablet, a Januvia® 50 mg core tablet, or a Januvia® 100 mg core tablet. In another class of this embodiment, the core tablet further comprises a film coating. In another class of this embodiment, sitagliptin is sitagliptin dihydrogen phosphate. In another class of this embodiment, sitagliptin is sitagliptin dihydrogen phosphate monohydrate.

In another embodiment, a core tablet comprising sitagliptin prepared by the addition of an antioxidant deposited on an excipient wherein the NTTP level is less than or equal to 1 ppm. In a class of this embodiment, the core tablet prepared by the addition of an antioxidant deposited on an excipient wherein the NTTP level is less than or equal to 1 ppm after storing at 70 °C for 2.5 days. In another class of this embodiment, the core tablet prepared by the addition of an antioxidant deposited on an excipient wherein the NTTP level is less than or equal to 1 ppm after storing at 60 °C and 60% relative humidity for 1.5 weeks; after storing at 70 °C and 60% relative humidity for 2.5 days; after storing at 40 °C and 75% relative humidity for 1.5 months; or after storing at 40 °C and 65% relative humidity for 1.5 weeks. In another class of this embodiment, the antioxidant is propyl gallate and the excipient is dicalcium phosphate. In another class of this embodiment, the core tablet is a Januvia® 25 mg core tablet, a Januvia® 50 mg core tablet, or a Januvia® 100 mg core tablet. In another class of this embodiment, the core tablet further comprises a film coating. In another class of this embodiment, sitagliptin is sitagliptin dihydrogen phosphate. In another class of this embodiment, sitagliptin is sitagliptin dihydrogen phosphate monohydrate.

In another embodiment, a core tablet comprising sitagliptin prepared by the addition of an antioxidant deposited on an excipient wherein the NTTP level is less than or equal to 0.37 ppm. In a class of this embodiment, the core tablet prepared by the addition of an antioxidant deposited on an excipient wherein the NTTP level is less than or equal to 0.37 ppm after storing at 70 °C for 2.5 days. In another class of this embodiment, the core tablet prepared by the addition of an antioxidant deposited on an excipient wherein the NTTP level is less than or equal to 0.37 ppm after storing at 60 °C and 60% relative humidity for 1.5 weeks; after storing at 70 °C and 60% relative humidity for 2.5 days; after storing at 40 °C and 75% relative humidity for 1.5 months: or after storing at 40 °C and 65% relative humidity for 1.5 weeks. In another class of this embodiment, the antioxidant is propyl gallate and the excipient is dicalcium phosphate. In another class of this embodiment, the core tablet is a Januvia® 25 mg core tablet, a Januvia® 50 mg core tablet, or a Januvia® 100 mg core tablet. In another class of this embodiment, the core tablet further comprises a film coating. In another class of this embodiment, sitagliptin is sitagliptin dihydrogen phosphate. In another class of this embodiment, sitagliptin is sitagliptin dihydrogen phosphate monohydrate.

The present disclosure includes pharmaceutical compositions of sitagliptin, or a pharmaceutically acceptable salt thereof, ertugliflozin, or a salt or co-crystal thereof, and an antioxidant. The present disclosure also includes pharmaceutical compositions of sitagliptin dihydrogen phosphate monohydrate, ertugliflozin L-pyroglutamic acid co-crystal, and antioxidant. The pharmaceutical compositions may include additional excipients, including but not limited to, microcrystalline cellulose, dicalcium phosphate, croscarmellose sodium, magnesium stearate, and/or sodium stearyl fumarate. The pharmaceutical compositions may also include Opadry 20 and/or carnauba wax. The pharmaceutical compositions may be compressed into tablets and optionally film coated.

In one embodiment, the pharmaceutical composition comprises 6.48 mg ertugliflozin L-pyroglutamic acid and 128.5 mg of sitagliptin phosphate monohydrate which is equivalent to 5 mg of ertugliflozin and 100 mg of the sitagliptin free base. In another embodiment, the pharmaceutical composition comprises 19.43 mg ertugliflozin L-pyroglutamic acid and 128.5 mg of sitagliptin phosphate monohydrate, which is equivalent to 15 mg of ertugliflozin and 100 mg of the sitagliptin free base.

In another embodiment, the sitagliptin phosphate-antioxidant composition is used in the STEGLUJAN® pharmaceutical composition. In another embodiment, the sitagliptin dihydrogen phosphate monohydrate-antioxidant composition is used in the pharmaceutical composition for STEGLUJAN®. In another embodiment, the sitagliptin phosphate-antioxidant composition is used in STEGLUJAN® tablets. In another embodiment, the sitagliptin dihydrogen phosphate monohydrate-antioxidant composition is used in STEGLUJAN® tablets. In another embodiment, sitagliptin phosphate-antioxidant composition comprises sitagliptin dihydrogen phosphate and an antioxidant. In another embodiment, sitagliptin phosphate-antioxidant composition comprises sitagliptin dihydrogen phosphate and propyl gallate. In another embodiment, sitagliptin phosphate-antioxidant composition comprises sitagliptin dihydrogen phosphate monohydrate and propyl gallate. In another embodiment, the sitagliptin dihydrogen phosphate monohydrate-propyl gallate composition is used in STEGLUJAN® tablets. In another embodiment, sitagliptin phosphate-antioxidant composition comprises sitagliptin dihydrogen phosphate and butylated hydroxyanisole. In another embodiment, sitagliptin phosphate-antioxidant composition comprises sitagliptin dihydrogen phosphate monohydrate and butylated hydroxyanisole. In another embodiment, the sitagliptin dihydrogen phosphate monohydrate-butylated hydroxyanisole composition is used in STEGLUJAN® tablets.

In one embodiment, the pharmaceutical composition comprises:
a) 10-50 weight % of sitagliptin dihydrogen phosphate monohydrate;
b) 0.5-15 weight % ertugliflozin L-pyroglutamic acid;
c) 0.01-0.5 weight % antioxidant;
d) 10-50 weight % microcrystalline cellulose;
e) 10-50 weight % dibasic calcium phosphate anhydrous;
f) 0.5-5 weight % croscarmellose sodium;
g) 0.5-5 weight % sodium stearyl fumarate; and
h) 0.5-2 weight % magnesium stearate.

In a class of this embodiment, the antioxidant is butylated hydroxyanisole. In a subclass of this class, the particle size of butylated hydroxyanisole is ≤400 microns. In another subclass of this class, the particle size of butylated hydroxyanisole is 1 to 400 microns.

In another subclass of this class, the particle size of butylated hydroxyanisole is ≤375 microns.

In another subclass of this class, the particle size of butylated hydroxyanisole is 1 to 375 microns. In another subclass of this class, the particle size of butylated hydroxyanisole is 22 to 375 microns. In another subclass of this class, the particle size of butylated hydroxyanisole is ≤150 microns. In another subclass of this class, the particle size of butylated hydroxyanisole is 1 to 150 microns. In another subclass of this class, the particle size of butylated hydroxyanisole is ≤83 microns. In another subclass of this class the particle size of butylated hydroxyanisole is 1 to 83 microns. In another subclass of this class, the particle size of butylated hydroxyanisole is ≤44 microns. In another subclass of this class, the particle size of butylated hydroxyanisole is 1 to 44 microns. In another subclass of this class, the particle size of butylated hydroxyanisole is ≤22 microns. In another subclass of this class, the particle size of butylated hydroxyanisole is 1 to 22 microns.

In another class of this embodiment, the antioxidant is propyl gallate. In a subclass of this class, the particle size of propyl gallate is ≤400 microns. In another subclass of this class the particle size of propyl gallate is 1 to 400 microns. In another subclass of this class, the particle size of propyl gallate is ≤375 microns. In another subclass of this class, the particle size of propyl gallate is 1 to 375 microns. In another subclass of this class, the particle size of propyl gallate is 22 to 375 microns. In another subclass of this class, the particle size of propyl gallate is ≤150 microns. In another subclass of this class, the particle size of propyl gallate is 1 to 150 microns. In another subclass of this class, the particle size of propyl gallate is ≤83 microns. In another subclass of this class, the particle size of propyl gallate is 1 to 83 microns.

In another subclass of this class, the particle size of propyl gallate is ≤44 microns. In another subclass of this class, the particle size of propyl gallate is 1 to 44 microns. In another subclass of this class, the particle size of propyl gallate is ≤22 microns. In another subclass of this class, the particle size of propyl gallate is 1 to 22 microns.

In another class of this embodiment, the pharmaceutical composition is formed into a tablet. In a subclass of this class, the tablet is film coated.

In another embodiment, the pharmaceutical composition comprises:
a) 10-50 weight % of sitagliptin dihydrogen phosphate monohydrate;
b) 0.5-15 weight % ertugliflozin L-pyroglutamic acid;
c) 0.01-0.5 weight % antioxidant;
d) 10-50 weight % microcrystalline cellulose;
e) 10-50 weight % dibasic calcium phosphate anhydrous;
f) 0.5-5 weight % croscarmellose sodium;
g) 0.5-5 weight % sodium stearyl fumarate;
h) 0.5-2 weight % magnesium stearate; and
i) 1-6 weight % Opadry 20A.

In a class of this embodiment, the antioxidant is butylated hydroxyanisole. In a subclass of this class, the particle size of butylated hydroxyanisole is ≤400 microns. In another subclass of this class, the particle size of butylated hydroxyanisole is 1 to 400 microns. In another subclass of this class, the particle size of butylated hydroxyanisole is ≤375 microns. In another subclass of this class, the particle size of butylated hydroxyanisole is 1 to 375 microns. In another subclass of this class, the particle size of butylated hydroxyanisole is 22 to 375 microns. In another subclass of this class, the particle size of butylated hydroxyanisole is ≤150 microns. In another subclass of this class, the particle size of butylated hydroxyanisole is 1 to 150 microns. In another subclass of this class, the particle size of butylated hydroxyanisole is ≤83 microns. In another subclass of this class, the particle size of butylated hydroxyanisole is 1 to 83 microns. In another subclass of this class, the particle size of butylated hydroxyanisole is ≤44 microns. In another subclass of this class, the particle size of butylated hydroxyanisole is 1 to 44 microns. In another subclass of this class, the particle size of butylated hydroxyanisole is ≤22 microns. In another subclass of this class, the particle size of butylated hydroxyanisole is 1 to 22 microns.

In another class of this embodiment, the antioxidant is propyl gallate. In a subclass of this class, the particle size of propyl gallate is ≤400 microns. In another subclass of this class, the particle size of propyl gallate is 1 to 400 microns. In another subclass of this class, the particle size of propyl gallate is ≤375 microns. In another subclass of this class, the particle size of propyl gallate is 1 to 375 microns. In another subclass of this class, the particle size of propyl gallate is 22 to 375 microns. In another subclass of this class, the particle size of propyl gallate is ≤150 microns. In another subclass of this class, the particle size of propyl gallate is 1 to 150 microns. In another subclass of this class, the particle size of propyl gallate is ≤83 microns. In another subclass of this class, the particle size of propyl gallate is 1 to 83 microns. In another subclass of this class, the particle size of propyl gallate is ≤44 microns. In another subclass of this class, the particle size of propyl gallate is 1 to 44 microns. In another subclass of this class, the particle size of propyl gallate is ≤22 microns. In another subclass of this class, the particle size of propyl gallate is 1 to 22 microns.

In another class of this embodiment, the pharmaceutical composition is formed into a tablet. In a subclass of this class, the tablet is film coated.

In another embodiment, the pharmaceutical composition comprises:
a) 10-50 weight % of sitagliptin dihydrogen phosphate monohydrate;
b) 0.5-15 weight % ertugliflozin L-pyroglutamic acid;
c) 0.01-0.5 weight % antioxidant;
d) 10-50 weight % microcrystalline cellulose;
e) 10-50 weight % dibasic calcium phosphate anhydrous;
f) 0.5-5 weight % croscarmellose sodium;
g) 0.5-5 weight % sodium stearyl fumarate;
h) 0.5-2 weight % magnesium stearate;
i) 1-6 weight % Opadry 20A; and
j) 0.001-0.005 weight % carnauba wax.

In a class of this embodiment, the antioxidant is butylated hydroxyanisole. In a subclass of this class, the particle size of butylated hydroxyanisole is ≤400 microns. In another subclass of this class, the particle size of butylated hydroxyanisole is 1 to 400 microns. In another subclass of this class, the particle size of butylated hydroxyanisole is ≤375 microns. In another subclass of this class, the particle size of butylated hydroxyanisole is 1 to 375 microns. In another subclass of this class, the particle size of butylated hydroxyanisole is 22 to 375 microns. In another subclass of this class, the particle size of butylated hydroxyanisole is ≤150 microns. In another subclass of this class, the particle size of butylated hydroxyanisole is 1 to 150 microns. In another subclass of this class, the particle size of butylated hydroxyanisole is ≤83 microns. In another subclass of this class, the particle size of butylated hydroxyanisole is 1 to 83 microns. In another subclass of this class, the particle size of butylated hydroxyanisole is ≤44 microns. In another subclass of this class, the particle size of butylated hydroxyanisole is 1 to 44 microns. In another subclass of this class, the particle size of butylated hydroxyanisole is ≤22 microns. In another subclass of this class, the particle size of butylated hydroxyanisole is 1 to 22 microns.

In another class of this embodiment, the antioxidant is propyl gallate. In a subclass of this class, the particle size of propyl gallate is ≤400 microns. In another subclass of this class, the particle size of propyl gallate is 1 to 400 microns. In another subclass of this class, the particle size of propyl gallate is ≤375 microns. In another subclass of this class, the particle size of propyl gallate is 1 to 375 microns. In another subclass of this class, the particle size of propyl gallate is 22 to 375 microns. In another subclass of this class, the particle size of propyl gallate is ≤150 microns. In another subclass of this class, the particle size of propyl gallate is 1 to 150 microns. In another subclass of this class, the particle size of propyl gallate is ≤83 microns. In another subclass of this class, the particle size of propyl gallate is 1 to 83 microns. In another subclass of this class, the particle size of propyl gallate is ≤44 microns. In another subclass of this class, the particle size of propyl gallate is 1 to 44 microns. In another subclass of this class, the particle size of propyl gallate is ≤22 microns. In another subclass of this class, the particle size of propyl gallate is 1 to 22 microns.

In another class of this embodiment, the pharmaceutical composition is formed into a tablet. In a subclass of this class, the tablet is film coated.

In another embodiment, the sitagliptin dihydrogen phosphate monohydrate, ertugliflozin L-pyroglutamic acid, and antioxidant pharmaceutical composition is prepared by formation of a sitagliptin dihydrogen phosphate monohydrate-antioxidant composition or complex and addition of the composition or complex to the dry pharmaceutical composition ingredients prior to tablet formation. In a class of this embodiment, the antioxidant is butylated hydroxyanisole. In another class of this embodiment, the antioxidant is propyl gallate.

In another embodiment, the sitagliptin dihydrogen phosphate monohydrate, ertugliflozin L-pyroglutamic acid, and antioxidant pharmaceutical composition is prepared by addition of the antioxidant as a dry powder to the pharmaceutical composition of sitagliptin dihydrogen phosphate monohydrate prior to tablet formation.

In a class of this embodiment, the antioxidant is butylated hydroxyanisole. In a subclass of this class, the particle size of butylated hydroxyanisole is ≤400 microns. In another subclass of this class, the particle size of butylated hydroxyanisole is 1 to 400 microns. In another subclass of this class, the particle size of butylated hydroxyanisole is ≤375 microns. In another subclass of this class, the particle size of butylated hydroxyanisole is 1 to 375 microns. In another subclass of this class, the particle size of butylated hydroxyanisole is 22 to 375 microns. In another subclass of this class, the particle size of butylated hydroxyanisole is ≤150 microns. In another subclass of this class, the particle size of butylated hydroxyanisole is 1 to 150 microns. In another subclass of this class, the particle size of butylated hydroxyanisole is ≤83 microns. In another subclass of this class, the particle size of butylated hydroxyanisole is 1 to 83 microns. In another subclass of this class, the particle size of butylated hydroxyanisole is ≤44 microns. In another subclass of this class, the particle size of butylated hydroxyanisole is 1 to 44 microns. In another subclass of this class, the particle size of butylated hydroxyanisole is ≤22 microns. In another subclass of this class, the particle size of butylated hydroxyanisole is 1 to 22 microns.

In another class of this embodiment, the antioxidant is propyl gallate. In a subclass of this class, the particle size of propyl gallate is ≤400 microns. In another subclass of this class, the particle size of propyl gallate is 1 to 400 microns. In another subclass of this class, the particle size of propyl gallate is ≤375 microns. In another subclass of this class, the particle size of propyl gallate is 1 to 375 microns. In another subclass of this class, the particle size of propyl gallate is 22 to 375 microns. In another subclass of this class, the particle size of propyl gallate is ≤150 microns. In another subclass of this class, the particle size of propyl gallate is 1 to 150 microns. In another subclass of this class, the particle size of propyl gallate is ≤83 microns. In another subclass of this class, the particle size of propyl gallate is 1 to 83 microns. In another subclass of this class, the particle size of propyl gallate is ≤44 microns. In another subclass of this class, the particle size of propyl gallate is 1 to 44 microns. In another subclass of this class, the particle size of propyl gallate is ≤22 microns. In another subclass of this class, the particle size of propyl gallate is 1 to 22 microns.

In another embodiment, the sitagliptin dihydrogen phosphate monohydrate ertugliflozin L-pyroglutamic acid, and antioxidant pharmaceutical composition is prepared by deposition of the antioxidant on an excipient and addition of the antioxidant/excipient composition to the pharmaceutical composition of sitagliptin dihydrogen phosphate monohydrate prior to tablet formation. In a class of this embodiment, the antioxidant is butylated hydroxyanisole. In another class of this embodiment, the antioxidant is propyl gallate.

In another embodiment, a core tablet comprising sitagliptin and ertugliflozin prepared by the addition of the sitagliptin dihydrogen phosphate monohydrate antioxidant composition wherein the NTTP level is less than or equal to 4 ppm. In a class of this embodiment, the core tablet prepared by the addition of the sitagliptin dihydrogen phosphate monohydrate antioxidant composition wherein the NTTP level is less than or equal to 4 ppm after storing at 70 °C for 2.5 days. In another class of this embodiment, the core tablet prepared by the addition of the sitagliptin dihydrogen phosphate monohydrate antioxidant composition wherein the NTTP level is less than or equal to 4 ppm after storing at 70 °C for 2.5 days, or after storing at 60 °C and 30% relative humidity for 1.5 weeks. In a class of this embodiment, the antioxidant is propyl gallate or butylated hydroxyanisole. In another class of this embodiment, the antioxidant is propyl gallate. In another class of this embodiment, the core tablet is a Steglujan® 5 mg/100 mg core tablet, or a Steglujan® 15 mg/100 mg core tablet. In another class of this embodiment, the core tablet further comprises a film coating. In another class of this embodiment, ertugliflozin is ertugliflozin L-pyroglutamic acid.

In another embodiment, a core tablet comprising sitagliptin and ertugliflozin prepared by the addition of the sitagliptin dihydrogen phosphate monohydrate antioxidant composition wherein the NTTP level is less than or equal to 2 ppm. In a class of this embodiment, the core tablet prepared by the addition of the sitagliptin dihydrogen phosphate monohydrate antioxidant composition wherein the NTTP level is less than or equal to 2 ppm after storing at 70 °C for 2.5 days. In another class of this embodiment, the core tablet prepared by the addition of the sitagliptin dihydrogen phosphate monohydrate antioxidant composition wherein the NTTP level is less than or equal to 2 ppm after storing at 70 °C for 2.5 days, or after storing at 60 °C and 30% relative humidity for 1.5 weeks. In a class of this embodiment, the antioxidant is propyl gallate or butylated hydroxyanisole. In another class of this embodiment, the antioxidant is propyl gallate. In another class of this embodiment, the core tablet is a Steglujan® 5 mg/100 mg core tablet, or a Steglujan® 15 mg/100 mg core tablet. In another class of this embodiment, the core tablet further comprises a film coating. In another class of this embodiment, ertugliflozin is ertugliflozin L-pyroglutamic acid.

In another embodiment, a core tablet comprising sitagliptin and ertugliflozin prepared by the addition of the sitagliptin dihydrogen phosphate monohydrate antioxidant composition wherein the NTTP level is less than or equal to 1 ppm. In a class of this embodiment, the core tablet prepared by the addition of the sitagliptin dihydrogen phosphate monohydrate antioxidant composition wherein the NTTP level is less than or equal to 1 ppm after storing at 70 °C for 2.5 days. In another class of this embodiment, the core tablet prepared by the addition of the sitagliptin dihydrogen phosphate monohydrate antioxidant composition wherein the NTTP level is less than or equal to 1 ppm after storing at 70 °C for 2.5 days, or after storing at 60 °C and 30% relative humidity for 1.5 weeks. In a class of this embodiment, the antioxidant is propyl gallate or butylated hydroxyanisole. In another class of this embodiment, the antioxidant is propyl gallate. In another class of this embodiment, the core tablet is a Steglujan® 5 mg/100 mg core tablet, or a Steglujan® 15 mg/100 mg core tablet. In another class of this embodiment, the core tablet further comprises a film coating. In another class of this embodiment, ertugliflozin is ertugliflozin L-pyroglutamic acid.

In another embodiment, a core tablet comprising sitagliptin and ertugliflozin prepared by the addition of the sitagliptin dihydrogen phosphate monohydrate antioxidant composition wherein the NTTP level is less than or equal to 0.37 ppm. In a class of this embodiment, the core tablet prepared by the addition of the sitagliptin dihydrogen phosphate monohydrate antioxidant composition wherein the NTTP level is less than or equal to 0.37 ppm after storing at 70 °C for 2.5 days. In another class of this embodiment, the core tablet prepared by the addition of the sitagliptin dihydrogen phosphate monohydrate antioxidant composition wherein the NTTP level is less than or equal to 0.37 ppm after storing at 70 °C for 2.5 days, or after storing at 60 °C and 30% relative humidity for 1.5 weeks. In a class of this embodiment, the antioxidant is propyl gallate or butylated hydroxyanisole. In another class of this embodiment, the antioxidant is propyl gallate. In another class of this embodiment, the core tablet is a Steglujan® 5 mg/100 mg core tablet, or a Steglujan® 15 mg/100 mg core tablet. In another class of this embodiment, the core tablet further comprises a film coating. In another class of this embodiment, ertugliflozin is ertugliflozin L-pyroglutamic acid.

In another embodiment, a core tablet comprising sitagliptin and ertugliflozin prepared by the addition of an antioxidant deposited on an excipient wherein the NTTP level is less than or equal to 4 ppm. In a class of this embodiment, the core tablet prepared by the addition of an antioxidant deposited on an excipient wherein the NTTP level is less than or equal to 4 ppm after storing at 70 °C for 2.5 days. In another class of this embodiment, the core tablet prepared by the addition of an antioxidant deposited on an excipient, wherein the NTTP level is less than or equal to 4 ppm after storing at 70 °C for 2.5 days, or after storing at 60 °C and 30% relative humidity for 1.5 weeks. In another class of this embodiment, the antioxidant is propyl gallate and the excipient is dicalcium phosphate. In another class of this embodiment, the core tablet is a Steglujan® 5 mg/100 mg core tablet, or a Steglujan® 15 mg/100 mg core tablet. In another class of this embodiment, the core tablet further comprises a film coating. In another class of this embodiment, sitagliptin is sitagliptin dihydrogen phosphate. In another class of this embodiment, sitagliptin is sitagliptin dihydrogen phosphate monohydrate. In another class of this embodiment, ertugliflozin is ertugliflozin L-pyroglutamic acid.

In another embodiment, a core tablet comprising sitagliptin and ertugliflozin prepared by the addition of an antioxidant deposited on an excipient wherein the NTTP level is less than or equal to 2 ppm. In a class of this embodiment, the core tablet prepared by the addition of an antioxidant deposited on an excipient wherein the NTTP level is less than or equal to 2 ppm after storing at 70 °C for 2.5 days. In another class of this embodiment, the core tablet prepared by the addition of an antioxidant deposited on an excipient, wherein the NTTP level is less than or equal to 2 ppm after storing at 70 °C for 2.5 days, or after storing at 60 °C and 30% relative humidity for 1.5 weeks. In another class of this embodiment, the antioxidant is propyl gallate and the excipient is dicalcium phosphate. In another class of this embodiment, the core tablet is a Steglujan® 5 mg/100 mg core tablet, or a Steglujan® 15 mg/100 mg core tablet. In another class of this embodiment, the core tablet further comprises a film coating. In another class of this embodiment, sitagliptin is sitagliptin dihydrogen phosphate. In another class of this embodiment, sitagliptin is sitagliptin dihydrogen phosphate monohydrate. In another class of this embodiment, ertugliflozin is ertugliflozin L-pyroglutamic acid.

In another embodiment, a core tablet comprising sitagliptin and ertugliflozin prepared by the addition of an antioxidant deposited on an excipient wherein the NTTP level is less than or equal to 1 ppm. In a class of this embodiment, the core tablet prepared by the addition of an antioxidant deposited on an excipient wherein the NTTP level is less than or equal to 1 ppm after storing at 70 °C for 2.5 days. In another class of this embodiment, the core tablet prepared by the addition of an antioxidant deposited on an excipient, wherein the NTTP level is less than or equal to 1 ppm after storing at 70 °C for 2.5 days, or after storing at 60 °C and 30% relative humidity for 1.5 weeks. In another class of this embodiment, the antioxidant is propyl gallate and the excipient is dicalcium phosphate. In another class of this embodiment, the core tablet is a Steglujan® 5 mg/100 mg core tablet, or a Steglujan® 15 mg/100 mg core tablet. In another class of this embodiment, the core tablet further comprises a film coating. In another class of this embodiment, sitagliptin is sitagliptin dihydrogen phosphate. In another class of this embodiment, sitagliptin is sitagliptin dihydrogen phosphate monohydrate. In another class of this embodiment, ertugliflozin is ertugliflozin L-pyroglutamic acid.

In another embodiment, a core tablet comprising sitagliptin and ertugliflozin prepared by the addition of an antioxidant deposited on an excipient wherein the NTTP level is less than or equal to 0.37 ppm. In a class of this embodiment, the core tablet prepared by the addition of an antioxidant deposited on an excipient wherein the NTTP level is less than or equal to 0.37 ppm after storing at 70 °C for 2.5 days. In another class of this embodiment, the core tablet prepared by the addition of an antioxidant deposited on an excipient wherein the NTTP level is less than or equal to 0.37 ppm after storing at 70 °C for 2.5 days, or after storing at 60 °C and 30% relative humidity for 1.5 weeks. In another class of this embodiment, the antioxidant is propyl gallate and the excipient is dicalcium phosphate. In another class of this embodiment, the core tablet is a Steglujan® 5 mg/100 mg core tablet, or a Steglujan® 15 mg/100 mg core tablet. In another class of this embodiment, the core tablet further comprises a film coating. In another class of this embodiment, sitagliptin is sitagliptin dihydrogen phosphate. In another class of this embodiment, sitagliptin is sitagliptin dihydrogen phosphate monohydrate. In another class of this embodiment, ertugliflozin is ertugliflozin L-pyroglutamic acid.

In another embodiment, a core tablet comprising sitagliptin and ertugliflozin prepared by the addition of an antioxidant as a dry powder wherein the NTTP level is less than or equal to 4 ppm. In a class of this embodiment, the core tablet prepared by the addition of an antioxidant as a dry powder wherein the NTTP level is less than or equal to 4 ppm after storing at 70 °C for 2.5 days. In another class of this embodiment, the core tablet prepared by the addition of an antioxidant as a dry powder wherein the NTTP level is less than or equal to 4 ppm after storing at 70 °C for 2.5 days, or after storing at 60 °C and 30% relative humidity for 1.5 weeks. In another class of this embodiment, the antioxidant is propyl gallate or butylated hydroxyanisole. In another class of this embodiment, the antioxidant is propyl gallate. In another class of this embodiment, the core tablet is a Steglujan® 5 mg/100 mg core tablet, or a Steglujan® 15 mg/100 mg core tablet. In another class of this embodiment, the core tablet further comprises a film coating. In another class of this embodiment, sitagliptin is sitagliptin dihydrogen phosphate. In another class of this embodiment, sitagliptin is sitagliptin dihydrogen phosphate monohydrate. In another class of this embodiment, ertugliflozin is ertugliflozin L-pyroglutamic acid.

In another embodiment, a core tablet comprising sitagliptin and ertugliflozin prepared by the addition of an antioxidant as a dry powder wherein the NTTP level is less than or equal to 2 ppm. In a class of this embodiment, the core tablet prepared by the addition of an antioxidant as a dry powder wherein the NTTP level is less than or equal to 2 ppm after storing at 70 °C for 2.5 days. In another class of this embodiment, the core tablet prepared by the addition of an antioxidant as a dry powder wherein the NTTP level is less than or equal to 2 ppm after storing at 70 °C for 2.5 days, or after storing at 60 °C and 30% relative humidity for 1.5 weeks. In another class of this embodiment, the antioxidant is propyl gallate or butylated hydroxyanisole. In another class of this embodiment, the antioxidant is propyl gallate. In another class of this embodiment, the core tablet is a Steglujan® 5 mg/100 mg core tablet, or a Steglujan® 15 mg/100 mg core tablet. In another class of this embodiment, the core tablet further comprises a film coating. In another class of this embodiment, sitagliptin is sitagliptin dihydrogen phosphate. In another class of this embodiment, sitagliptin is sitagliptin dihydrogen phosphate monohydrate. In another class of this embodiment, ertugliflozin is ertugliflozin L-pyroglutamic acid.

In another embodiment, a core tablet comprising sitagliptin and ertugliflozin prepared by the addition of an antioxidant as a dry powder wherein the NTTP level is less than or equal to 1 ppm. In a class of this embodiment, the core tablet prepared by the addition of an antioxidant as a dry powder wherein the NTTP level is less than or equal to 1 ppm after storing at 70 °C for 2.5 days. In another class of this embodiment, the core tablet prepared by the addition of an antioxidant as a dry powder wherein the NTTP level is less than or equal to 1 ppm after storing at 70 °C for 2.5 days, or after storing at 60 °C and 30% relative humidity for 1.5 weeks. In another class of this embodiment, the antioxidant is propyl gallate or butylated hydroxyanisole. In another class of this embodiment, the antioxidant is propyl gallate. In another class of this embodiment, the core tablet is a Steglujan® 5 mg/100 mg core tablet, or a Steglujan® 15 mg/100 mg core tablet. In another class of this embodiment, the core tablet further comprises a film coating. In another class of this embodiment, sitagliptin is sitagliptin dihydrogen phosphate. In another class of this embodiment, sitagliptin is sitagliptin dihydrogen phosphate monohydrate. In another class of this embodiment, ertugliflozin is ertugliflozin L-pyroglutamic acid.

In another embodiment, a core tablet comprising sitagliptin and ertugliflozin prepared by the addition of an antioxidant as a dry powder wherein the NTTP level is less than or equal to 0.37 ppm. In a class of this embodiment, the core tablet prepared by the addition of an antioxidant as a dry powder wherein the NTTP level is less than or equal to 0.37 ppm after storing at 70 °C for 2.5 days. In another class of this embodiment, the core tablet prepared by the addition of an antioxidant as a dry powder wherein the NTTP level is less than or equal to 0.37 ppm after storing at 70 °C for 2.5 days, or after storing at 60 °C and 30% relative humidity for 1.5 weeks. In another class of this embodiment, the antioxidant is propyl gallate or butylated hydroxyanisole. In another class of this embodiment, the antioxidant is propyl gallate. In another class of this embodiment, the core tablet is a Steglujan® 5 mg/100 mg core tablet, or a Steglujan® 15 mg/100 mg core tablet. In another class of this embodiment, the core tablet further comprises a film coating. In another class of this embodiment, sitagliptin is sitagliptin dihydrogen phosphate. In another class of this embodiment, sitagliptin is sitagliptin dihydrogen phosphate monohydrate. In another class of this embodiment, ertugliflozin is ertugliflozin L-pyroglutamic acid.

In another embodiment, a pharmaceutical composition comprising sitagliptin, microcrystalline cellulose, and an antioxidant wherein the NTTP level is less than or equal to 4 ppm. In a class of this embodiment, the pharmaceutical composition comprising sitagliptin, microcrystalline cellulose, and an antioxidant wherein the NTTP level is less than or equal to 4 ppm after storing at 70 °C for 2.5 days. In another class of this embodiment, the pharmaceutical composition comprising sitagliptin, microcrystalline cellulose, and an antioxidant wherein the NTTP level is less than or equal to 4 ppm after storing at 70 °C for 2.5 days; after storing at 60 °C and 60% relative humidity for 1.5 weeks; after storing at 70 °C and 60% relative humidity for 2.5 days; after storing at 40 °C and 75% relative humidity for 1.5 months; or after storing at 40 °C and 65% relative humidity for 1.5 weeks. In another class of this embodiment, the antioxidant is propyl gallate or butylated hydroxyanisole. In another class of this embodiment, the antioxidant is propyl gallate. In another class of this embodiment, the pharmaceutical composition is prepared by 1) the addition of the sitagliptin dihydrogen phosphate monohydrate antioxidant composition; 2) the addition of an antioxidant as a dry powder; or 3) the addition of an antioxidant deposited on an excipient. In a subclass of this class, the antioxidant is propyl gallate, and the excipient is dicalcium phosphate. In another class of this embodiment, sitagliptin is sitagliptin dihydrogen phosphate. In another class of this embodiment, sitagliptin is sitagliptin dihydrogen phosphate monohydrate. In another class of this embodiment, the pharmaceutical composition further comprises ertugliflozin. In another class of this embodiment, the pharmaceutical composition further comprises ertugliflozin L-pyroglutamic acid.

In another embodiment, a pharmaceutical composition comprising sitagliptin, microcrystalline cellulose, and an antioxidant wherein the NTTP level is less than or equal to 1 ppm. In a class of this embodiment, the pharmaceutical composition comprising sitagliptin, microcrystalline cellulose, and an antioxidant wherein the NTTP level is less than or equal to 1 ppm after storing at 70 °C for 2.5 days. In another class of this embodiment, the pharmaceutical composition comprising sitagliptin, microcrystalline cellulose, and an antioxidant wherein the NTTP level is less than or equal to 1 ppm after storing at 70 °C for 2.5 days; after storing at 60 °C and 60% relative humidity for 1.5 weeks; after storing at 70 °C and 60% relative humidity for 2.5 days; after storing at 40 °C and 75% relative humidity for 1.5 months; or after storing at 40 °C and 65% relative humidity for 1.5 weeks. In another class of this embodiment, the antioxidant is propyl gallate or butylated hydroxyanisole. In another class of this embodiment, the antioxidant is propyl gallate. In another class of this embodiment, the pharmaceutical composition is prepared by 1) the addition of the sitagliptin dihydrogen phosphate monohydrate antioxidant composition; 2) the addition of an antioxidant as a dry powder; or 3) the addition of an antioxidant deposited on an excipient. In a subclass of this class, the antioxidant is propyl gallate, and the excipient is dicalcium phosphate. In another class of this embodiment, sitagliptin is sitagliptin dihydrogen phosphate. In another class of this embodiment, sitagliptin is sitagliptin dihydrogen phosphate monohydrate. In another class of this embodiment, the pharmaceutical composition further comprises ertugliflozin. In another class of this embodiment, the pharmaceutical composition further comprises ertugliflozin L-pyroglutamic acid.

In another embodiment, a pharmaceutical composition comprising sitagliptin, microcrystalline cellulose, and an antioxidant wherein the NTTP level is less than or equal to 0.37 ppm. In a class of this embodiment, the pharmaceutical composition comprising sitagliptin, microcrystalline cellulose, and an antioxidant wherein the NTTP level is less than or equal to 0.37 ppm after storing at 70 °C for 2.5 days. In another class of this embodiment, the pharmaceutical composition comprising sitagliptin, microcrystalline cellulose, and an antioxidant wherein the NTTP level is less than or equal to 0.37 ppm after storing at 70 °C for 2.5 days; after storing at 60 °C and 60% relative humidity for 1.5 weeks; after storing at 70 °C and 60% relative humidity for 2.5 days; after storing at 40 °C and 75% relative humidity for 1.5 months; or after storing at 40 °C and 65% relative humidity for 1.5 weeks. In another class of this embodiment, the antioxidant is propyl gallate or butylated hydroxyanisole. In another class of this embodiment, the antioxidant is propyl gallate. In another class of this embodiment, The pharmaceutical composition is prepared by 1) the addition of the sitagliptin dihydrogen phosphate monohydrate antioxidant composition; 2) the addition of an antioxidant as a dry powder; or 3) the addition of an antioxidant deposited on an excipient. In a subclass of this class, the antioxidant is propyl gallate, and the excipient is dicalcium phosphate. In another class of this embodiment, sitagliptin is sitagliptin dihydrogen phosphate. In another class of this embodiment, sitagliptin is sitagliptin dihydrogen phosphate monohydrate. In another class of this embodiment, the pharmaceutical composition further comprises ertugliflozin. In another class of this embodiment, the pharmaceutical composition further comprises ertugliflozin L-pyroglutamic acid.

In another embodiment, a pharmaceutical composition comprising sitagliptin, dicalcium phosphate, and an antioxidant wherein the NTTP level is less than or equal to 4 ppm. In a class of this embodiment, the pharmaceutical composition comprising sitagliptin, dicalcium phosphate, and an antioxidant wherein the NTTP level is less than or equal to 4 ppm after storing at 70 °C for 2.5 days. In another class of this embodiment, the pharmaceutical composition comprising sitagliptin, dicalcium phosphate, and an antioxidant wherein the NTTP level is less than or equal to 4 ppm after storing at 70 °C for 2.5 days; after storing at 60 °C and 60% relative humidity for 1.5 weeks; after storing at 70 °C and 60% relative humidity for 2.5 days; after storing at 40 °C and 75% relative humidity for 1.5 months; or after storing at 40 °C and 65% relative humidity for 1.5 weeks. In another class of this embodiment, the antioxidant is propyl gallate or butylated hydroxyanisole. In another class of this embodiment, the antioxidant is propyl gallate. In another class of this embodiment, the pharmaceutical composition is prepared by 1) the addition of the sitagliptin dihydrogen phosphate monohydrate antioxidant composition; 2) the addition of an antioxidant as a dry powder; or 3) the addition of an antioxidant deposited on an excipient. In a subclass of this class, the antioxidant is propyl gallate, and the excipient is dicalcium phosphate. In another class of this embodiment, sitagliptin is sitagliptin dihydrogen phosphate. In another class of this embodiment, sitagliptin is sitagliptin dihydrogen phosphate monohydrate. In another class of this embodiment, the pharmaceutical composition further comprises ertugliflozin. In another class of this embodiment, the pharmaceutical composition further comprises ertugliflozin L-pyroglutamic acid.

In another embodiment, a pharmaceutical composition comprising sitagliptin, dicalcium phosphate, and an antioxidant wherein the NTTP level is less than or equal to 1 ppm. In a class of this embodiment, the pharmaceutical composition comprising sitagliptin, dicalcium phosphate, and an antioxidant wherein the NTTP level is less than or equal to 1 ppm after storing at 70 °C for 2.5 days. In another class of this embodiment, the pharmaceutical composition comprising sitagliptin, dicalcium phosphate, and an antioxidant wherein the NTTP level is less than or equal to 1 ppm after storing at 70 °C for 2.5 days; after storing at 60 °C and 60% relative humidity for 1.5 weeks; after storing at 70 °C and 60% relative humidity for 2.5 days; after storing at 40 °C and 75% relative humidity for 1.5 months; or after storing at 40 °C and 65% relative humidity for 1.5 weeks. In another class of this embodiment, the antioxidant is propyl gallate or butylated hydroxyanisole. In another class of this embodiment, the antioxidant is propyl gallate. In another class of this embodiment, the pharmaceutical composition is prepared by 1) the addition of the sitagliptin dihydrogen phosphate monohydrate antioxidant composition; 2) the addition of an antioxidant as a dry powder; or 3) the addition of an antioxidant deposited on an excipient. In a subclass of this class, the antioxidant is propyl gallate, and the excipient is dicalcium phosphate. In another class of this embodiment, sitagliptin is sitagliptin dihydrogen phosphate. In another class of this embodiment, sitagliptin is sitagliptin dihydrogen phosphate monohydrate. In another class of this embodiment, the pharmaceutical composition further comprises ertugliflozin. In another class of this embodiment, the pharmaceutical composition further comprises ertugliflozin L-pyroglutamic acid.

In another embodiment, a pharmaceutical composition comprising sitagliptin, dicalcium phosphate, and an antioxidant wherein the NTTP level is less than or equal to 0.37 ppm. In a class of this embodiment, the pharmaceutical composition comprising sitagliptin, dicalcium phosphate, and an antioxidant wherein the NTTP level is less than or equal to 0.37 ppm after storing at 70 °C for 2.5 days. In another class of this embodiment, the pharmaceutical composition comprising sitagliptin, dicalcium phosphate, and an antioxidant wherein the NTTP level is less than or equal to 0.37 ppm after storing at 70 °C for 2.5 days; after storing at 60 °C and 60% relative humidity for 1.5 weeks; after storing at 70 °C and 60% relative humidity for 2.5 days; after storing at 40 °C and 75% relative humidity for 1.5 months; or after storing at 40 °C and 65% relative humidity for 1.5 weeks. In another class of this embodiment, the antioxidant is propyl gallate or butylated hydroxyanisole. In another class of this embodiment, the antioxidant is propyl gallate. In another class of this embodiment, the pharmaceutical composition is prepared by 1) the addition of the sitagliptin dihydrogen phosphate monohydrate antioxidant composition; 2) the addition of an antioxidant as a dry powder; or 3) the addition of an antioxidant deposited on an excipient. In a subclass of this class, the antioxidant is propyl gallate and the excipient is dicalcium phosphate. In another class of this embodiment, sitagliptin is sitagliptin dihydrogen phosphate. In another class of this embodiment, sitagliptin is sitagliptin dihydrogen phosphate monohydrate. In another class of this embodiment, the pharmaceutical composition further comprises ertugliflozin. In another class of this embodiment, the pharmaceutical composition further comprises ertugliflozin L-pyroglutamic acid.

Specific embodiments of the sitagliptin-antioxidant composition include but are not limited to:

A composition comprising:
(a) sitagliptin, or a pharmaceutically acceptable salt and/or hydrate thereof; and
(b) an antioxidant.

In one embodiment, the pharmaceutically acceptable salt of sitagliptin is the dihydrogen phosphate salt of structural formula I above (sitagliptin phosphate). In another embodiment, the pharmaceutically acceptable hydrate of sitagliptin is the monohydrate. In another embodiment the pharmaceutically acceptable salt and hydrate of sitagliptin is the dihydrogen phosphate monohydrate. In another embodiment, the form of the dihydrogen phosphate salt is the crystalline monohydrate disclosed in U.S. Pat. No. 7,326,708, the contents of which are hereby incorporated by reference in their entirety. In another embodiment, the pharmaceutically acceptable salt and hydrate of sitagliptin is sitagliptin dihydrogen phosphate monohydrate salt. In another embodiment, the pharmaceutically acceptable salt and hydrate of sitagliptin is the crystalline sitagliptin dihydrogen phosphate monohydrate.

In one embodiment, the antioxidant is selected from the group: ascorbic acid butylated hydroxytoluene, methyl gallate, ethyl gallate, propyl gallate, butyl gallate, pentyl gallate octyl gallate, dodecyl (lauryl) gallate, butylated hydroxyanisole, thymol, tocopherol, and vitamin E. In another embodiment, the antioxidant is selected from the group: ascorbic acid, butylated hydroxytoluene, propyl gallate, butyl gallate, butylated hydroxyanisole, thymol, tocopherol, and vitamin E. In another embodiment, the antioxidant is selected from: propyl gallate and butylated hydroxyanisole. In another embodiment, the antioxidant is propyl gallate. In another embodiment, the antioxidant is butylated hydroxyanisole.

A composition consisting essentially of:
(a) sitagliptin, or a pharmaceutically acceptable salt and/or hydrate thereof; and
(b) an antioxidant.

In one embodiment, the pharmaceutically acceptable salt of sitagliptin is the dihydrogen phosphate salt of structural formula I above (sitagliptin phosphate). In another embodiment, the pharmaceutically acceptable hydrate of sitagliptin is the monohydrate. In another embodiment the pharmaceutically acceptable salt and hydrate of sitagliptin is the dihydrogen phosphate monohydrate. In another embodiment, the form of the dihydrogen phosphate salt is the crystalline monohydrate disclosed in U.S. Pat. No. 7,326,708, the contents of which are hereby incorporated by reference in their entirety. In another embodiment, the pharmaceutically acceptable salt and hydrate of sitagliptin is sitagliptin dihydrogen phosphate monohydrate salt. In another embodiment, the pharmaceutically acceptable salt and hydrate of sitagliptin is the crystalline sitagliptin dihydrogen phosphate monohydrate.

In one embodiment, the antioxidant is selected from the group: ascorbic acid butylated hydroxytoluene, methyl gallate, ethyl gallate, propyl gallate, butyl gallate, pentyl gallate, octyl gallate, dodecyl (lauryl) gallate, butylated hydroxyanisole, thymol, tocopherol, and vitamin E. In another embodiment, the antioxidant is selected from the group: ascorbic acid, butylated hydroxytoluene, propyl gallate, butyl gallate, butylated hydroxyanisole, thymol, tocopherol, and vitamin E. In another embodiment, the antioxidant is selected from: propyl gallate and butylated hydroxyanisole. In another embodiment, the antioxidant is propyl gallate. In another embodiment, the antioxidant is butylated hydroxyanisole.

A composition comprising:
(a) about 90% to 99.99% by weight of a sitagliptin, or a pharmaceutically acceptable salt and/or hydrate thereof; and
(b) about 0.01% to 10% by weight of an antioxidant.

In one embodiment, the pharmaceutically acceptable salt of sitagliptin is the dihydrogen phosphate salt of structural formula I above (sitagliptin phosphate). In another embodiment, the pharmaceutically acceptable hydrate of sitagliptin is the monohydrate. In another embodiment the pharmaceutically acceptable salt and hydrate of sitagliptin is the dihydrogen phosphate monohydrate. In another embodiment, the form of the dihydrogen phosphate salt is the crystalline monohydrate disclosed in U.S. Pat. No. 7,326,708, the contents of which are hereby incorporated by reference in their entirety. In another embodiment, the pharmaceutically acceptable salt and hydrate of sitagliptin is sitagliptin dihydrogen phosphate monohydrate salt. In another embodiment, the pharmaceutically acceptable salt and hydrate of sitagliptin is the crystalline sitagliptin dihydrogen phosphate monohydrate.

In one embodiment, the antioxidant is selected from the group: ascorbic acid, butylated hydroxytoluene, methyl gallate, ethyl gallate, propyl gallate, butyl gallate, pentyl gallate, octyl gallate, dodecyl (lauryl) gallate, butylated hydroxyanisole, thymol, tocopherol, and vitamin E. In another embodiment, the antioxidant is selected from the group: ascorbic acid, butylated hydroxytoluene, propyl gallate, butyl gallate, butylated hydroxyanisole, thymol, tocopherol, and vitamin E. In another embodiment, the antioxidant is selected from: propyl gallate and butylated hydroxyanisole. In another embodiment, the antioxidant is propyl gallate. In another embodiment, the antioxidant is butylated hydroxyanisole.

A composition consisting essentially of
(a) about 90% to 99.99% by weight of a sitagliptin, or a pharmaceutically acceptable salt and/or hydrate thereof; and
(b) about 0.01% to 10% by weight of an antioxidant.

In one embodiment, the pharmaceutically acceptable salt of sitagliptin is the dihydrogen phosphate salt of structural formula I above (sitagliptin phosphate). In another embodiment, the pharmaceutically acceptable hydrate of sitagliptin is the monohydrate. In another embodiment the pharmaceutically acceptable salt and hydrate of sitagliptin is the dihydrogen phosphate monohydrate. In another embodiment, the form of the dihydrogen phosphate salt is the crystalline monohydrate disclosed in U.S. Pat. No. 7,326,708, the contents of which are hereby incorporated by reference in their entirety. In another embodiment, the pharmaceutically acceptable salt and hydrate of sitagliptin is sitagliptin dihydrogen phosphate monohydrate salt. In another embodiment, the pharmaceutically acceptable salt and hydrate of sitagliptin is the crystalline sitagliptin dihydrogen phosphate monohydrate.

In one embodiment, the antioxidant is selected from the group: ascorbic acid, butylated hydroxytoluene, methyl gallate, ethyl gallate, propyl gallate, butyl gallate, pentyl gallate octyl gallate dodecyl (lauryl) gallate, butylated hydroxyanisole, thymol, tocopherol, and vitamin E. In another embodiment, the antioxidant is selected from the group: ascorbic acid, butylated hydroxytoluene, propyl gallate, butyl gallate, butylated hydroxyanisole, thymol, tocopherol, and vitamin E. In another embodiment, the antioxidant is selected from: propyl gallate and butylated hydroxyanisole. In another embodiment, the antioxidant is propyl gallate. In another embodiment, the antioxidant is butylated hydroxyanisole.

A composition comprising:
(a) about 90% to 99.95% by weight of a sitagliptin, or a pharmaceutically acceptable salt and/or hydrate thereof; and
(b) about 0.05% to 10% by weight of an antioxidant.

In one embodiment, the pharmaceutically acceptable salt of sitagliptin is the dihydrogen phosphate salt of structural formula I above (sitagliptin phosphate). In another embodiment, the pharmaceutically acceptable hydrate of sitagliptin is the monohydrate. In another embodiment the pharmaceutically acceptable salt and hydrate of sitagliptin is the dihydrogen phosphate monohydrate. In another embodiment, the form of the dihydrogen phosphate salt is the crystalline monohydrate disclosed in U.S. Pat. No. 7,326,708, the contents of which are hereby incorporated by reference in their entirety. In another embodiment, the pharmaceutically acceptable salt and hydrate of sitagliptin is sitagliptin dihydrogen phosphate monohydrate salt. In another embodiment, the pharmaceutically acceptable salt and hydrate of sitagliptin is the crystalline sitagliptin dihydrogen phosphate monohydrate.

In one embodiment, the antioxidant is selected from the group: ascorbic acid butylated hydroxytoluene, methyl gallate, ethyl gallate, propyl gallate, butyl gallate, pentyl gallate octyl gallate, dodecyl (lauryl) gallate, butylated hydroxyanisole, thymol, tocopherol, and vitamin E. In another embodiment, the antioxidant is selected from the group: ascorbic acid, butylated hydroxytoluene, propyl gallate, butyl gallate, butylated hydroxyanisole, thymol, tocopherol, and vitamin E. In another embodiment, the antioxidant is selected from: propyl gallate and butylated hydroxyanisole. In another embodiment, the antioxidant is propyl gallate. In another embodiment, the antioxidant is butylated hydroxyanisole.

A composition consisting essentially of
(a) about 90% to 99.95% by weight of a sitagliptin, or a pharmaceutically acceptable salt and/or hydrate thereof; and
(b) about 0.05% to 10% by weight of an antioxidant.

In one embodiment, the pharmaceutically acceptable salt of sitagliptin is the dihydrogen phosphate salt of structural formula I above (sitagliptin phosphate). In another embodiment, the pharmaceutically acceptable hydrate of sitagliptin is the monohydrate. In another embodiment the pharmaceutically acceptable salt and hydrate of sitagliptin is the dihydrogen phosphate monohydrate. In another embodiment, the form of the dihydrogen phosphate salt is the crystalline monohydrate disclosed in U.S. Pat. No. 7,326,708, the contents of which are hereby incorporated by reference in their entirety. In another embodiment, the pharmaceutically acceptable salt and hydrate of sitagliptin is sitagliptin dihydrogen phosphate monohydrate salt. In another embodiment, the pharmaceutically acceptable salt and hydrate of sitagliptin is the crystalline sitagliptin dihydrogen phosphate monohydrate.

In one embodiment, the antioxidant is selected from the group: ascorbic acid, butylated hydroxytoluene, methyl gallate, ethyl gallate, propyl gallate, butyl gallate, pentyl gallate, octyl gallate, dodecyl (lauryl) gallate, butylated hydroxyanisole, thymol, tocopherol, and vitamin E. In another embodiment, the antioxidant is selected from the group: ascorbic acid, butylated hydroxytoluene, propyl gallate, butyl gallate, butylated hydroxyanisole, thymol, tocopherol, and vitamin E. In another embodiment, the antioxidant is selected from: propyl gallate and butylated hydroxyanisole. In another embodiment, the antioxidant is propyl gallate. In another embodiment, the antioxidant is butylated hydroxyanisole.

A composition comprising:
(a) about 90% to 99.9% by weight of a sitagliptin, or a pharmaceutically acceptable salt and/or hydrate thereof; and
(b) about 0.1% to 10% by weight of an antioxidant.

In one embodiment, the pharmaceutically acceptable salt of sitagliptin is the dihydrogen phosphate salt of structural formula I above (sitagliptin phosphate). In another embodiment, the pharmaceutically acceptable hydrate of sitagliptin is the monohydrate. In another embodiment the pharmaceutically acceptable salt and hydrate of sitagliptin is the dihydrogen phosphate monohydrate. In another embodiment, the form of the dihydrogen phosphate salt is the crystalline monohydrate disclosed in U.S. Pat. No. 7,326,708, the contents of which are hereby incorporated by reference in their entirety. In another embodiment, the pharmaceutically acceptable salt and hydrate of sitagliptin is sitagliptin dihydrogen phosphate monohydrate salt. In another embodiment, the pharmaceutically acceptable salt and hydrate of sitagliptin is the crystalline sitagliptin dihydrogen phosphate monohydrate.

In one embodiment, the antioxidant is selected from the group: ascorbic acid, butylated hydroxytoluene, methyl gallate, ethyl gallate, propyl gallate, butyl gallate, pentyl gallate octyl gallate dodecyl (lauryl) gallate, butylated hydroxyanisole, thymol, tocopherol, and vitamin E. In another embodiment, the antioxidant is selected from the group: ascorbic acid, butylated hydroxytoluene, propyl gallate, butyl gallate, butylated hydroxyanisole, thymol, tocopherol, and vitamin E. In another embodiment, the antioxidant is selected from: propyl gallate and butylated hydroxyanisole. In another embodiment, the antioxidant is propyl gallate. In another embodiment, the antioxidant is butylated hydroxyanisole.

A composition consisting essentially of:
(a) about 90% to 99.9% by weight of a sitagliptin, or a pharmaceutically acceptable salt and/or hydrate thereof; and
(b) about 0.1% to 10% by weight of an antioxidant.

In one embodiment, the pharmaceutically acceptable salt of sitagliptin is the dihydrogen phosphate salt of structural formula I above (sitagliptin phosphate). In another embodiment, the pharmaceutically acceptable hydrate of sitagliptin is the monohydrate. In another embodiment the pharmaceutically acceptable salt and hydrate of sitagliptin is the dihydrogen phosphate monohydrate. In another embodiment, the form of the dihydrogen phosphate salt is the crystalline monohydrate disclosed in U.S. Pat. No. 7,326,708, the contents of which are hereby incorporated by reference in their entirety. In another embodiment, the pharmaceutically acceptable salt and hydrate of sitagliptin is sitagliptin dihydrogen phosphate monohydrate salt. In another embodiment, the pharmaceutically acceptable salt and hydrate of sitagliptin is the crystalline sitagliptin dihydrogen phosphate monohydrate.

In one embodiment, the antioxidant is selected from the group: ascorbic acid butylated hydroxytoluene, methyl gallate, ethyl gallate, propyl gallate, butyl gallate, pentyl gallate octyl gallate, dodecyl (lauryl) gallate, butylated hydroxyanisole, thymol, tocopherol, and vitamin E. In another embodiment, the antioxidant is selected from the group: ascorbic acid, butylated hydroxytoluene, propyl gallate, butyl gallate, butylated hydroxyanisole, thymol, tocopherol, and vitamin E. In another embodiment, the antioxidant is selected from: propyl gallate and butylated hydroxyanisole. In another embodiment, the antioxidant is propyl gallate. In another embodiment, the antioxidant is butylated hydroxyanisole.

A composition comprising:
(a) about 95% to 99.99% by weight of a sitagliptin, or a pharmaceutically acceptable salt and/or hydrate thereof; and
(b) about 0.01% to 5% by weight of an antioxidant.

In one embodiment, the pharmaceutically acceptable salt of sitagliptin is the dihydrogen phosphate salt of structural formula I above (sitagliptin phosphate). In another embodiment, the pharmaceutically acceptable hydrate of sitagliptin is the monohydrate. In another embodiment the pharmaceutically acceptable salt and hydrate of sitagliptin is the dihydrogen phosphate monohydrate. In another embodiment, the form of the dihydrogen phosphate salt is the crystalline monohydrate disclosed in U.S. Pat. No. 7,326,708, the contents of which are hereby incorporated by reference in their entirety. In another embodiment, the pharmaceutically acceptable salt and hydrate of sitagliptin is sitagliptin dihydrogen phosphate monohydrate salt. In another embodiment, the pharmaceutically acceptable salt and hydrate of sitagliptin is the crystalline sitagliptin dihydrogen phosphate monohydrate.

In one embodiment, the antioxidant is selected from the group: ascorbic acid, butylated hydroxytoluene, methyl gallate, ethyl gallate, propyl gallate, butyl gallate, pentyl gallate, octyl gallate, dodecyl (lauryl) gallate, butylated hydroxyanisole, thymol, tocopherol, and vitamin E. In another embodiment, the antioxidant is selected from the group: ascorbic acid, butylated hydroxytoluene, propyl gallate, butyl gallate, butylated hydroxyanisole, thymol, tocopherol, and vitamin E. In another embodiment, the antioxidant is selected from: propyl gallate and butylated hydroxyanisole. In another embodiment, the antioxidant is propyl gallate. In another embodiment, the antioxidant is butylated hydroxyanisole.

A composition consisting essentially of
(a) about 95% to 99.99% by weight of a sitagliptin, or a pharmaceutically acceptable salt and/or hydrate thereof; and
(b) about 0.01% to 5% by weight of an antioxidant.

In one embodiment, the pharmaceutically acceptable salt of sitagliptin is the dihydrogen phosphate salt of structural formula I above (sitagliptin phosphate). In another embodiment, the pharmaceutically acceptable hydrate of sitagliptin is the monohydrate. In another embodiment the pharmaceutically acceptable salt and hydrate of sitagliptin is the dihydrogen phosphate monohydrate. In another embodiment, the form of the dihydrogen phosphate salt is the crystalline monohydrate disclosed in U.S. Pat. No. 7,326,708, the contents of which are hereby incorporated by reference in their entirety. In another embodiment, the pharmaceutically acceptable salt and hydrate of sitagliptin is sitagliptin dihydrogen phosphate monohydrate salt. In another embodiment, the pharmaceutically acceptable salt and hydrate of sitagliptin is the crystalline sitagliptin dihydrogen phosphate monohydrate.

In one embodiment, the antioxidant is selected from the group: ascorbic acid, butylated hydroxytoluene, methyl gallate, ethyl gallate, propyl gallate, butyl gallate, pentyl gallate octyl gallate dodecyl (lauryl) gallate, butylated hydroxyanisole, thymol, tocopherol, and vitamin E. In another embodiment, the antioxidant is selected from the group: ascorbic acid, butylated hydroxytoluene, propyl gallate, butyl gallate, butylated hydroxyanisole, thymol, tocopherol, and vitamin E. In another embodiment, the antioxidant is selected from: propyl gallate and butylated hydroxyanisole. In another embodiment, the antioxidant is propyl gallate. In another embodiment, the antioxidant is butylated hydroxyanisole.

A composition comprising:
(a) about 95% to 99.95% by weight of a sitagliptin, or a pharmaceutically acceptable salt and/or hydrate thereof; and
(b) about 0.05% to 5% by weight of an antioxidant.

In one embodiment, the pharmaceutically acceptable salt of sitagliptin is the dihydrogen phosphate salt of structural formula I above (sitagliptin phosphate). In another embodiment, the pharmaceutically acceptable hydrate of sitagliptin is the monohydrate. In another embodiment the pharmaceutically acceptable salt and hydrate of sitagliptin is the dihydrogen phosphate monohydrate. In another embodiment, the form of the dihydrogen phosphate salt is the crystalline monohydrate disclosed in U.S. Pat. No. 7,326,708, the contents of which are hereby incorporated by reference in their entirety. In another embodiment, the pharmaceutically acceptable salt and hydrate of sitagliptin is sitagliptin dihydrogen phosphate monohydrate salt. In another embodiment, the pharmaceutically acceptable salt and hydrate of sitagliptin is the crystalline sitagliptin dihydrogen phosphate monohydrate.

In one embodiment, the antioxidant is selected from the group: ascorbic acid butylated hydroxytoluene, methyl gallate, ethyl gallate, propyl gallate, butyl gallate, pentyl gallate octyl gallate, dodecyl (lauryl) gallate, butylated hydroxyanisole, thymol, tocopherol, and vitamin E. In another embodiment, the antioxidant is selected from the group: ascorbic acid, butylated hydroxytoluene, propyl gallate, butyl gallate, butylated hydroxyanisole, thymol, tocopherol, and vitamin E. In another embodiment, the antioxidant is selected from: propyl gallate and butylated hydroxyanisole. In another embodiment, the antioxidant is propyl gallate. In another embodiment, the antioxidant is butylated hydroxyanisole.

A composition consisting essentially of
(a) about 95% to 99.95% by weight of a sitagliptin, or a pharmaceutically acceptable salt and/or hydrate thereof; and
(b) about 0.05% to 5% by weight of an antioxidant.

In one embodiment, the pharmaceutically acceptable salt of sitagliptin is the dihydrogen phosphate salt of structural formula I above (sitagliptin phosphate). In another embodiment, the pharmaceutically acceptable hydrate of sitagliptin is the monohydrate. In another embodiment the pharmaceutically acceptable salt and hydrate of sitagliptin is the dihydrogen phosphate monohydrate. In another embodiment, the form of the dihydrogen phosphate salt is the crystalline monohydrate disclosed in U.S. Pat. No. 7,326,708, the contents of which are hereby incorporated by reference in their entirety. In another embodiment, the pharmaceutically acceptable salt and hydrate of sitagliptin is sitagliptin dihydrogen phosphate monohydrate salt. In another embodiment, the pharmaceutically acceptable salt and hydrate of sitagliptin is the crystalline sitagliptin dihydrogen phosphate monohydrate.

In one embodiment, the antioxidant is selected from the group: ascorbic acid, butylated hydroxytoluene, methyl gallate, ethyl gallate, propyl gallate, butyl gallate, pentyl gallate, octyl gallate, dodecyl (lauryl) gallate, butylated hydroxyanisole, thymol, tocopherol, and vitamin E. In another embodiment, the antioxidant is selected from the group: ascorbic acid, butylated hydroxytoluene, propyl gallate, butyl gallate, butylated hydroxyanisole, thymol, tocopherol, and vitamin E. In another embodiment, the antioxidant is selected from: propyl gallate and butylated hydroxyanisole. In another embodiment, the antioxidant is propyl gallate. In another embodiment, the antioxidant is butylated hydroxyanisole.

A composition comprising:
(a) about 95% to 99.9% by weight of a sitagliptin, or a pharmaceutically acceptable salt and/or hydrate thereof; and
(b) about 0.1% to 5% by weight of an antioxidant.

In one embodiment, the pharmaceutically acceptable salt of sitagliptin is the dihydrogen phosphate salt of structural formula I above (sitagliptin phosphate). In another embodiment, the pharmaceutically acceptable hydrate of sitagliptin is the monohydrate. In another embodiment the pharmaceutically acceptable salt and hydrate of sitagliptin is the dihydrogen phosphate monohydrate. In another embodiment, the form of the dihydrogen phosphate salt is the crystalline monohydrate disclosed in U.S. Pat. No. 7,326,708, the contents of which are hereby incorporated by reference in their entirety. In another embodiment, the pharmaceutically acceptable salt and hydrate of sitagliptin is sitagliptin dihydrogen phosphate monohydrate salt. In another embodiment, the pharmaceutically acceptable salt and hydrate of sitagliptin is the crystalline sitagliptin dihydrogen phosphate monohydrate.

In one embodiment, the antioxidant is selected from the group: ascorbic acid, butylated hydroxytoluene, methyl gallate, ethyl gallate, propyl gallate, butyl gallate, pentyl gallate octyl gallate dodecyl (lauryl) gallate, butylated hydroxyanisole, thymol, tocopherol, and vitamin E. In another embodiment, the antioxidant is selected from the group: ascorbic acid, butylated hydroxytoluene, propyl gallate, butyl gallate, butylated hydroxyanisole, thymol, tocopherol, and vitamin E. In another embodiment, the antioxidant is selected from: propyl gallate and butylated hydroxyanisole. In another embodiment, the antioxidant is propyl gallate. In another embodiment, the antioxidant is butylated hydroxyanisole.

A composition consisting essentially of
(a) about 95% to 99.9% by weight of a sitagliptin, or a pharmaceutically acceptable salt and/or hydrate thereof; and
(b) about 0.1% to 5% by weight of an antioxidant.

In one embodiment, the pharmaceutically acceptable salt of sitagliptin is the dihydrogen phosphate salt of structural formula I above (sitagliptin phosphate). In another embodiment, the pharmaceutically acceptable hydrate of sitagliptin is the monohydrate. In another embodiment the pharmaceutically acceptable salt and hydrate of sitagliptin is the dihydrogen phosphate monohydrate. In another embodiment, the form of the dihydrogen phosphate salt is the crystalline monohydrate disclosed in U.S. Pat. No. 7,326,708, the contents of which are hereby incorporated by reference in their entirety. In another embodiment, the pharmaceutically acceptable salt and hydrate of sitagliptin is sitagliptin dihydrogen phosphate monohydrate salt. In another embodiment, the pharmaceutically acceptable salt and hydrate of sitagliptin is the crystalline sitagliptin dihydrogen phosphate monohydrate.

In one embodiment, the antioxidant is selected from the group: ascorbic acid butylated hydroxytoluene, methyl gallate, ethyl gallate, propyl gallate, butyl gallate, pentyl gallate octyl gallate, dodecyl (lauryl) gallate, butylated hydroxyanisole, thymol, tocopherol, and vitamin E. In another embodiment, the antioxidant is selected from the group: ascorbic acid, butylated hydroxytoluene, propyl gallate, butyl gallate, butylated hydroxyanisole, thymol, tocopherol, and vitamin E. In another embodiment, the antioxidant is selected from: propyl gallate and butylated hydroxyanisole. In another embodiment, the antioxidant is propyl gallate. In another embodiment, the antioxidant is butylated hydroxyanisole.

A composition comprising:
(a) about 98% to 99.99% by weight of a sitagliptin, or a pharmaceutically acceptable salt and/or hydrate thereof; and
(b) about 0.01% to 2% by weight of an antioxidant.

In one embodiment, the pharmaceutically acceptable salt of sitagliptin is the dihydrogen phosphate salt of structural formula I above (sitagliptin phosphate). In another embodiment, the pharmaceutically acceptable hydrate of sitagliptin is the monohydrate. In another embodiment the pharmaceutically acceptable salt and hydrate of sitagliptin is the dihydrogen phosphate monohydrate. In another embodiment, the form of the dihydrogen phosphate salt is the crystalline monohydrate disclosed in U.S. Pat. No. 7,326,708, the contents of which are hereby incorporated by reference in their entirety. In another embodiment, the pharmaceutically acceptable salt and hydrate of sitagliptin is sitagliptin dihydrogen phosphate monohydrate salt. In another embodiment, the pharmaceutically acceptable salt and hydrate of sitagliptin is the crystalline sitagliptin dihydrogen phosphate monohydrate.

In one embodiment, the antioxidant is selected from the group: ascorbic acid, butylated hydroxytoluene, methyl gallate, ethyl gallate, propyl gallate, butyl gallate, pentyl gallate, octyl gallate, dodecyl (lauryl) gallate, butylated hydroxyanisole, thymol, tocopherol, and vitamin E. In another embodiment, the antioxidant is selected from the group: ascorbic acid, butylated hydroxytoluene, propyl gallate, butyl gallate, butylated hydroxyanisole, thymol, tocopherol, and vitamin E. In another embodiment, the antioxidant is selected from: propyl gallate and butylated hydroxyanisole. In another embodiment, the antioxidant is propyl gallate. In another embodiment, the antioxidant is butylated hydroxyanisole.

A composition consisting essentially of
(a) about 98% to 99.99% by weight of a sitagliptin, or a pharmaceutically acceptable salt and/or hydrate thereof; and
(b) about 0.01% to 2% by weight of an antioxidant.

In one embodiment, the pharmaceutically acceptable salt of sitagliptin is the dihydrogen phosphate salt of structural formula I above (sitagliptin phosphate). In another embodiment, the pharmaceutically acceptable hydrate of sitagliptin is the monohydrate. In another embodiment the pharmaceutically acceptable salt and hydrate of sitagliptin is the dihydrogen phosphate monohydrate. In another embodiment, the form of the dihydrogen phosphate salt is the crystalline monohydrate disclosed in U.S. Pat. No. 7,326,708, the contents of which are hereby incorporated by reference in their entirety. In another embodiment, the pharmaceutically acceptable salt and hydrate of sitagliptin is sitagliptin dihydrogen phosphate monohydrate salt. In another embodiment, the pharmaceutically acceptable salt and hydrate of sitagliptin is the crystalline sitagliptin dihydrogen phosphate monohydrate.

In one embodiment, the antioxidant is selected from the group: ascorbic acid, butylated hydroxytoluene, methyl gallate, ethyl gallate, propyl gallate, butyl gallate, pentyl gallate octyl gallate dodecyl (lauryl) gallate, butylated hydroxyanisole, thymol, tocopherol, and vitamin E. In another embodiment, the antioxidant is selected from the group: ascorbic acid, butylated hydroxytoluene, propyl gallate, butyl gallate, butylated hydroxyanisole, thymol, tocopherol, and vitamin E. In another embodiment, the antioxidant is selected from: propyl gallate and butylated hydroxyanisole. In another embodiment, the antioxidant is propyl gallate. In another embodiment, the antioxidant is butylated hydroxyanisole.

A composition comprising:
(a) about 98% to 99.9% by weight of a sitagliptin, or a pharmaceutically acceptable salt and/or hydrate thereof; and
(b) about 0.1% to 2% by weight of an antioxidant.

In one embodiment, the pharmaceutically acceptable salt of sitagliptin is the dihydrogen phosphate salt of structural formula I above (sitagliptin phosphate). In another embodiment, the pharmaceutically acceptable hydrate of sitagliptin is the monohydrate. In another embodiment the pharmaceutically acceptable salt and hydrate of sitagliptin is the dihydrogen phosphate monohydrate. In another embodiment, the form of the dihydrogen phosphate salt is the crystalline monohydrate disclosed in U.S. Pat. No. 7,326,708, the contents of which are hereby incorporated by reference in their entirety. In another embodiment, the pharmaceutically acceptable salt and hydrate of sitagliptin is sitagliptin dihydrogen phosphate monohydrate salt. In another embodiment, the pharmaceutically acceptable salt and hydrate of sitagliptin is the crystalline sitagliptin dihydrogen phosphate monohydrate.

In one embodiment, the antioxidant is selected from the group: ascorbic acid butylated hydroxytoluene, methyl gallate, ethyl gallate, propyl gallate, butyl gallate, pentyl gallate octyl gallate, dodecyl (lauryl) gallate, butylated hydroxyanisole, thymol, tocopherol, and vitamin E. In another embodiment, the antioxidant is selected from the group: ascorbic acid, butylated hydroxytoluene, propyl gallate, butyl gallate, butylated hydroxyanisole, thymol, tocopherol, and vitamin E. In another embodiment, the antioxidant is selected from: propyl gallate and butylated hydroxyanisole. In another embodiment, the antioxidant is propyl gallate. In another embodiment, the antioxidant is butylated hydroxyanisole.

A composition consisting essentially of
(a) about 98% to 99.9% by weight of a sitagliptin, or a pharmaceutically acceptable salt and/or hydrate thereof; and
(b) about 0.1% to 2% by weight of an antioxidant.

In one embodiment, the pharmaceutically acceptable salt of sitagliptin is the dihydrogen phosphate salt of structural formula I above (sitagliptin phosphate). In another embodiment, the pharmaceutically acceptable hydrate of sitagliptin is the monohydrate. In another embodiment the pharmaceutically acceptable salt and hydrate of sitagliptin is the dihydrogen phosphate monohydrate. In another embodiment, the form of the dihydrogen phosphate salt is the crystalline monohydrate disclosed in U.S. Pat. No. 7,326,708, the contents of which are hereby incorporated by reference in their entirety. In another embodiment, the pharmaceutically acceptable salt and hydrate of sitagliptin is sitagliptin dihydrogen phosphate monohydrate salt. In another embodiment, the pharmaceutically acceptable salt and hydrate of sitagliptin is the crystalline sitagliptin dihydrogen phosphate monohydrate.

In one embodiment, the antioxidant is selected from the group: ascorbic acid, butylated hydroxytoluene, methyl gallate, ethyl gallate, propyl gallate, butyl gallate, pentyl gallate, octyl gallate, dodecyl (lauryl) gallate, butylated hydroxyanisole, thymol, tocopherol, and vitamin E. In another embodiment, the antioxidant is selected from the group: ascorbic acid, butylated hydroxytoluene, propyl gallate, butyl gallate, butylated hydroxyanisole, thymol, tocopherol, and vitamin E. In another embodiment, the antioxidant is selected from: propyl gallate and butylated hydroxyanisole. In another embodiment, the antioxidant is propyl gallate. In another embodiment, the antioxidant is butylated hydroxyanisole.

A composition comprising:
(a) about 99% to 99.99% by weight of a sitagliptin, or a pharmaceutically acceptable salt and/or hydrate thereof; and
(b) about 0.01% to 1% by weight of an antioxidant.

In one embodiment, the pharmaceutically acceptable salt of sitagliptin is the dihydrogen phosphate salt of structural formula I above (sitagliptin phosphate). In another embodiment, the pharmaceutically acceptable hydrate of sitagliptin is the monohydrate. In another embodiment the pharmaceutically acceptable salt and hydrate of sitagliptin is the dihydrogen phosphate monohydrate. In another embodiment, the form of the dihydrogen phosphate salt is the crystalline monohydrate disclosed in U.S. Pat. No. 7,326,708, the contents of which are hereby incorporated by reference in their entirety. In another embodiment, the pharmaceutically acceptable salt and hydrate of sitagliptin is sitagliptin dihydrogen phosphate monohydrate salt. In another embodiment, the pharmaceutically acceptable salt and hydrate of sitagliptin is the crystalline sitagliptin dihydrogen phosphate monohydrate.

In one embodiment, the antioxidant is selected from the group: ascorbic acid, butylated hydroxytoluene, methyl gallate, ethyl gallate, propyl gallate, butyl gallate, pentyl gallate octyl gallate dodecyl (lauryl) gallate, butylated hydroxyanisole, thymol, tocopherol, and vitamin E. In another embodiment, the antioxidant is selected from the group: ascorbic acid, butylated hydroxytoluene, propyl gallate, butyl gallate, butylated hydroxyanisole, thymol, tocopherol, and vitamin E. In another embodiment, the antioxidant is selected from: propyl gallate and butylated hydroxyanisole. In another embodiment, the antioxidant is propyl gallate. In another embodiment, the antioxidant is butylated hydroxyanisole.

A composition consisting essentially of
(a) about 99% to 99.99% by weight of a sitagliptin, or a pharmaceutically acceptable salt and/or hydrate thereof; and
(b) about 0.01% to 1% by weight of an antioxidant.

In one embodiment, the pharmaceutically acceptable salt of sitagliptin is the dihydrogen phosphate salt of structural formula I above (sitagliptin phosphate). In another embodiment, the pharmaceutically acceptable hydrate of sitagliptin is the monohydrate. In another embodiment the pharmaceutically acceptable salt and hydrate of sitagliptin is the dihydrogen phosphate monohydrate. In another embodiment, the form of the dihydrogen phosphate salt is the crystalline monohydrate disclosed in U.S. Pat. No. 7,326,708, the contents of which are hereby incorporated by reference in their entirety. In another embodiment, the pharmaceutically acceptable salt and hydrate of sitagliptin is sitagliptin dihydrogen phosphate monohydrate salt. In another embodiment, the pharmaceutically acceptable salt and hydrate of sitagliptin is the crystalline sitagliptin dihydrogen phosphate monohydrate.

In one embodiment, the antioxidant is selected from the group: ascorbic acid butylated hydroxytoluene, methyl gallate, ethyl gallate, propyl gallate, butyl gallate, pentyl gallate octyl gallate, dodecyl (lauryl) gallate, butylated hydroxyanisole, thymol, tocopherol, and vitamin E. In another embodiment, the antioxidant is selected from the group: ascorbic acid, butylated hydroxytoluene, propyl gallate, butyl gallate, butylated hydroxyanisole, thymol, tocopherol, and vitamin E. In another embodiment, the antioxidant is selected from: propyl gallate and butylated hydroxyanisole. In another embodiment, the antioxidant is propyl gallate. In another embodiment, the antioxidant is butylated hydroxyanisole.

A composition comprising:
(a) about 99.3% to 99.8% by weight of a sitagliptin, or a pharmaceutically acceptable salt and/or hydrate thereof; and
(b) about 0.2% to 0.7% by weight of an antioxidant.

In one embodiment, the pharmaceutically acceptable salt of sitagliptin is the dihydrogen phosphate salt of structural formula I above (sitagliptin phosphate). In another embodiment, the pharmaceutically acceptable hydrate of sitagliptin is the monohydrate. In another embodiment the pharmaceutically acceptable salt and hydrate of sitagliptin is the dihydrogen phosphate monohydrate. In another embodiment, the form of the dihydrogen phosphate salt is the crystalline monohydrate disclosed in U.S. Pat. No. 7,326,708, the contents of which are hereby incorporated by reference in their entirety. In another embodiment, the pharmaceutically acceptable salt and hydrate of sitagliptin is sitagliptin dihydrogen phosphate monohydrate salt. In another embodiment, the pharmaceutically acceptable salt and hydrate of sitagliptin is the crystalline sitagliptin dihydrogen phosphate monohydrate.

In one embodiment, the antioxidant is selected from the group: ascorbic acid, butylated hydroxytoluene, methyl gallate, ethyl gallate, propyl gallate, butyl gallate, pentyl gallate, octyl gallate, dodecyl (lauryl) gallate, butylated hydroxyanisole, thymol, tocopherol, and vitamin E. In another embodiment, the antioxidant is selected from the group: ascorbic acid, butylated hydroxytoluene, propyl gallate, butyl gallate, butylated hydroxyanisole, thymol, tocopherol, and vitamin E. In another embodiment, the antioxidant is selected from: propyl gallate and butylated hydroxyanisole. In another embodiment, the antioxidant is propyl gallate. In another embodiment, the antioxidant is butylated hydroxyanisole.

A composition consisting essentially of
(a) about 99.3% to 99.8% by weight of a sitagliptin, or a pharmaceutically acceptable salt and/or hydrate thereof; and
(b) about 0.2% to 0.7% by weight of an antioxidant.

In one embodiment, the pharmaceutically acceptable salt of sitagliptin is the dihydrogen phosphate salt of structural formula I above (sitagliptin phosphate). In another embodiment, the pharmaceutically acceptable hydrate of sitagliptin is the monohydrate. In another embodiment the pharmaceutically acceptable salt and hydrate of sitagliptin is the dihydrogen phosphate monohydrate. In another embodiment, the form of the dihydrogen phosphate salt is the crystalline monohydrate disclosed in U.S. Pat. No. 7,326,708, the contents of which are hereby incorporated by reference in their entirety. In another embodiment, the pharmaceutically acceptable salt and hydrate of sitagliptin is sitagliptin dihydrogen phosphate monohydrate salt. In another embodiment, the pharmaceutically acceptable salt and hydrate of sitagliptin is the crystalline sitagliptin dihydrogen phosphate monohydrate.

In one embodiment, the antioxidant is selected from the group: ascorbic acid, butylated hydroxytoluene, methyl gallate, ethyl gallate, propyl gallate, butyl gallate, pentyl gallate octyl gallate dodecyl (lauryl) gallate, butylated hydroxyanisole, thymol, tocopherol, and vitamin E. In another embodiment, the antioxidant is selected from the group: ascorbic acid, butylated hydroxytoluene, propyl gallate, butyl gallate, butylated hydroxyanisole, thymol, tocopherol, and vitamin E. In another embodiment, the antioxidant is selected from: propyl gallate and butylated hydroxyanisole. In another embodiment, the antioxidant is propyl gallate. In another embodiment, the antioxidant is butylated hydroxyanisole.

A composition comprising:
(a) about 99.60% to 99.75% by weight of a sitagliptin or a pharmaceutically acceptable salt and/or hydrate thereof; and
(b) about 0.25% to 0.40% by weight of an antioxidant.

In one embodiment, the pharmaceutically acceptable salt of sitagliptin is the dihydrogen phosphate salt of structural formula I above (sitagliptin phosphate). In another embodiment, the pharmaceutically acceptable hydrate of sitagliptin is the monohydrate. In another embodiment the pharmaceutically acceptable salt and hydrate of sitagliptin is the dihydrogen phosphate monohydrate. In another embodiment, the form of the dihydrogen phosphate salt is the crystalline monohydrate disclosed in U.S. Pat. No. 7,326,708, the contents of which are hereby incorporated by reference in their entirety. In another embodiment, the pharmaceutically acceptable salt and hydrate of sitagliptin is sitagliptin dihydrogen phosphate monohydrate salt. In another embodiment, the pharmaceutically acceptable salt and hydrate of sitagliptin is the crystalline sitagliptin dihydrogen phosphate monohydrate.

In one embodiment, the antioxidant is selected from the group: ascorbic acid butylated hydroxytoluene, methyl gallate, ethyl gallate, propyl gallate, butyl gallate, pentyl gallate octyl gallate, dodecyl (lauryl) gallate, butylated hydroxyanisole, thymol, tocopherol, and vitamin E. In another embodiment, the antioxidant is selected from the group: ascorbic acid, butylated hydroxytoluene, propyl gallate, butyl gallate, butylated hydroxyanisole, thymol, tocopherol, and vitamin E. In another embodiment, the antioxidant is selected from: propyl gallate and butylated hydroxyanisole. In another embodiment, the antioxidant is propyl gallate. In another embodiment, the antioxidant is butylated hydroxyanisole.

A composition consisting essentially of
(a) about 99.60% to 99.75% by weight of a sitagliptin, or a pharmaceutically acceptable salt and/or hydrate thereof; and
(b) about 0.25% to 0.40% by weight of an antioxidant.

In one embodiment, the pharmaceutically acceptable salt of sitagliptin is the dihydrogen phosphate salt of structural formula I above (sitagliptin phosphate). In another embodiment, the pharmaceutically acceptable hydrate of sitagliptin is the monohydrate. In another embodiment the pharmaceutically acceptable salt and hydrate of sitagliptin is the dihydrogen phosphate monohydrate. In another embodiment, the form of the dihydrogen phosphate salt is the crystalline monohydrate disclosed in U.S. Pat. No. 7,326,708, the contents of which are hereby incorporated by reference in their entirety. In another embodiment, the pharmaceutically acceptable salt and hydrate of sitagliptin is sitagliptin dihydrogen phosphate monohydrate salt. In another embodiment, the pharmaceutically acceptable salt and hydrate of sitagliptin is the crystalline sitagliptin dihydrogen phosphate monohydrate.

In one embodiment, the antioxidant is selected from the group: ascorbic acid, butylated hydroxytoluene, methyl gallate, ethyl gallate, propyl gallate, butyl gallate, pentyl gallate, octyl gallate, dodecyl (lauryl) gallate, butylated hydroxyanisole, thymol, tocopherol, and vitamin E. In another embodiment, the antioxidant is selected from the group: ascorbic acid, butylated hydroxytoluene, propyl gallate, butyl gallate, butylated hydroxyanisole, thymol, tocopherol, and vitamin E. In another embodiment, the antioxidant is selected from: propyl gallate and butylated hydroxyanisole. In another embodiment, the antioxidant is propyl gallate. In another embodiment, the antioxidant is butylated hydroxyanisole.

A composition comprising:
(a) about 99% to 99.9% by weight of a sitagliptin, or a pharmaceutically acceptable salt and/or hydrate thereof; and
(b) about 0.1% to 1% by weight of an antioxidant.

In one embodiment, the pharmaceutically acceptable salt of sitagliptin is the dihydrogen phosphate salt of structural formula I above (sitagliptin phosphate). In another embodiment, the pharmaceutically acceptable hydrate of sitagliptin is the monohydrate. In another embodiment the pharmaceutically acceptable salt and hydrate of sitagliptin is the dihydrogen phosphate monohydrate. In another embodiment, the form of the dihydrogen phosphate salt is the crystalline monohydrate disclosed in U.S. Pat. No. 7,326,708, the contents of which are hereby incorporated by reference in their entirety. In another embodiment, the pharmaceutically acceptable salt and hydrate of sitagliptin is sitagliptin dihydrogen phosphate monohydrate salt. In another embodiment, the pharmaceutically acceptable salt and hydrate of sitagliptin is the crystalline sitagliptin dihydrogen phosphate monohydrate.

In one embodiment, the antioxidant is selected from the group: ascorbic acid, butylated hydroxytoluene, methyl gallate, ethyl gallate, propyl gallate, butyl gallate, pentyl gallate octyl gallate dodecyl (lauryl) gallate, butylated hydroxyanisole, thymol, tocopherol, and vitamin E. In another embodiment, the antioxidant is selected from the group: ascorbic acid, butylated hydroxytoluene, propyl gallate, butyl gallate, butylated hydroxyanisole, thymol, tocopherol, and vitamin E. In another embodiment, the antioxidant is selected from: propyl gallate and butylated hydroxyanisole. In another embodiment, the antioxidant is propyl gallate. In another embodiment, the antioxidant is butylated hydroxyanisole.

A composition consisting essentially of
(a) about 99% to 99.9% by weight of a sitagliptin, or a pharmaceutically acceptable salt and/or hydrate thereof; and
(b) about 0.1% to 1% by weight of an antioxidant.

In one embodiment, the pharmaceutically acceptable salt of sitagliptin is the dihydrogen phosphate salt of structural formula I above (sitagliptin phosphate). In another embodiment, the pharmaceutically acceptable hydrate of sitagliptin is the monohydrate. In another embodiment the pharmaceutically acceptable salt and hydrate of sitagliptin is the dihydrogen phosphate monohydrate. In another embodiment, the form of the dihydrogen phosphate salt is the crystalline monohydrate disclosed in U.S. Pat. No. 7,326,708, the contents of which are hereby incorporated by reference in their entirety. In another embodiment, the pharmaceutically acceptable salt and hydrate of sitagliptin is sitagliptin dihydrogen phosphate monohydrate salt. In another embodiment, the pharmaceutically acceptable salt and hydrate of sitagliptin is the crystalline sitagliptin dihydrogen phosphate monohydrate.

In one embodiment, the antioxidant is selected from the group: ascorbic acid butylated hydroxytoluene, methyl gallate, ethyl gallate, propyl gallate, butyl gallate, pentyl gallate octyl gallate, dodecyl (lauryl) gallate, butylated hydroxyanisole, thymol, tocopherol, and vitamin E. In another embodiment, the antioxidant is selected from the group: ascorbic acid, butylated hydroxytoluene, propyl gallate, butyl gallate, butylated hydroxyanisole, thymol, tocopherol, and vitamin E. In another embodiment, the antioxidant is selected from: propyl gallate and butylated hydroxyanisole. In another embodiment, the antioxidant is propyl gallate. In another embodiment, the antioxidant is butylated hydroxyanisole.

A composition comprising:
(a) about 99.2% to 99.9% by weight of a sitagliptin, or a pharmaceutically acceptable salt and/or hydrate thereof; and
(b) about 0.1% to 0.8% by weight of an antioxidant.

In one embodiment, the pharmaceutically acceptable salt of sitagliptin is the dihydrogen phosphate salt of structural formula I above (sitagliptin phosphate). In another embodiment, the pharmaceutically acceptable hydrate of sitagliptin is the monohydrate. In another embodiment the pharmaceutically acceptable salt and hydrate of sitagliptin is the dihydrogen phosphate monohydrate. In another embodiment, the form of the dihydrogen phosphate salt is the crystalline monohydrate disclosed in U.S. Pat. No. 7,326,708, the contents of which are hereby incorporated by reference in their entirety. In another embodiment, the pharmaceutically acceptable salt and hydrate of sitagliptin is sitagliptin dihydrogen phosphate monohydrate salt. In another embodiment, the pharmaceutically acceptable salt and hydrate of sitagliptin is the crystalline sitagliptin dihydrogen phosphate monohydrate.

In one embodiment, the antioxidant is selected from the group: ascorbic acid, butylated hydroxytoluene, methyl gallate, ethyl gallate, propyl gallate, butyl gallate, pentyl gallate, octyl gallate, dodecyl (lauryl) gallate, butylated hydroxyanisole, thymol, tocopherol, and vitamin E. In another embodiment, the antioxidant is selected from the group: ascorbic acid, butylated hydroxytoluene, propyl gallate, butyl gallate, butylated hydroxyanisole, thymol, tocopherol, and vitamin E. In another embodiment, the antioxidant is selected from: propyl gallate and butylated hydroxyanisole. In another embodiment, the antioxidant is propyl gallate. In another embodiment, the antioxidant is butylated hydroxyanisole.

A composition consisting essentially of
(a) about 99.2% to 99.9% by weight of a sitagliptin, or a pharmaceutically acceptable salt and/or hydrate thereof; and
(b) about 0.1% to 0.8% by weight of an antioxidant.

In one embodiment, the pharmaceutically acceptable salt of sitagliptin is the dihydrogen phosphate salt of structural formula I above (sitagliptin phosphate). In another embodiment, the pharmaceutically acceptable hydrate of sitagliptin is the monohydrate. In another embodiment the pharmaceutically acceptable salt and hydrate of sitagliptin is the dihydrogen phosphate monohydrate. In another embodiment, the form of the dihydrogen phosphate salt is the crystalline monohydrate disclosed in U.S. Pat. No. 7,326,708, the contents of which are hereby incorporated by reference in their entirety. In another embodiment, the pharmaceutically acceptable salt and hydrate of sitagliptin is sitagliptin dihydrogen phosphate monohydrate salt. In another embodiment, the pharmaceutically acceptable salt and hydrate of sitagliptin is the crystalline sitagliptin dihydrogen phosphate monohydrate.

In one embodiment, the antioxidant is selected from the group: ascorbic acid, butylated hydroxytoluene, methyl gallate, ethyl gallate, propyl gallate, butyl gallate, pentyl gallate octyl gallate dodecyl (lauryl) gallate, butylated hydroxyanisole, thymol, tocopherol, and vitamin E. In another embodiment, the antioxidant is selected from the group: ascorbic acid, butylated hydroxytoluene, propyl gallate, butyl gallate, butylated hydroxyanisole, thymol, tocopherol, and vitamin E. In another embodiment, the antioxidant is selected from: propyl gallate and butylated hydroxyanisole. In another embodiment, the antioxidant is propyl gallate. In another embodiment, the antioxidant is butylated hydroxyanisole.

A composition comprising:
(a) about 90% by weight of a sitagliptin, or a pharmaceutically acceptable salt and/or hydrate thereof; and
(b) about 10% by weight of an antioxidant.

In one embodiment, the pharmaceutically acceptable salt of sitagliptin is the dihydrogen phosphate salt of structural formula I above (sitagliptin phosphate). In another embodiment, the pharmaceutically acceptable hydrate of sitagliptin is the monohydrate. In another embodiment the pharmaceutically acceptable salt and hydrate of sitagliptin is the dihydrogen phosphate monohydrate. In another embodiment, the form of the dihydrogen phosphate salt is the crystalline monohydrate disclosed in U.S. Pat. No. 7,326,708, the contents of which are hereby incorporated by reference in their entirety. In another embodiment, the pharmaceutically acceptable salt and hydrate of sitagliptin is sitagliptin dihydrogen phosphate monohydrate salt. In another embodiment, the pharmaceutically acceptable salt and hydrate of sitagliptin is the crystalline sitagliptin dihydrogen phosphate monohydrate.

In one embodiment, the antioxidant is selected from the group: ascorbic acid butylated hydroxytoluene, methyl gallate, ethyl gallate, propyl gallate, butyl gallate, pentyl gallate, octyl gallate, dodecyl (lauryl) gallate, butylated hydroxyanisole, thymol, tocopherol, and vitamin E. In another embodiment, the antioxidant is selected from the group: ascorbic acid, butylated hydroxytoluene, propyl gallate, butyl gallate, butylated hydroxyanisole, thymol, tocopherol, and vitamin E. In another embodiment, the antioxidant is selected from: propyl gallate and butylated hydroxyanisole. In another embodiment, the antioxidant is propyl gallate. In another embodiment, the antioxidant is butylated hydroxyanisole.

A composition consisting essentially of
(a) about 90% by weight of a sitagliptin, or a pharmaceutically acceptable salt and/or hydrate thereof; and
(b) about 10% by weight of an antioxidant.

In one embodiment, the pharmaceutically acceptable salt of sitagliptin is the dihydrogen phosphate salt of structural formula I above (sitagliptin phosphate). In another embodiment, the pharmaceutically acceptable hydrate of sitagliptin is the monohydrate. In another embodiment the pharmaceutically acceptable salt and hydrate of sitagliptin is the dihydrogen phosphate monohydrate. In another embodiment, the form of the dihydrogen phosphate salt is the crystalline monohydrate disclosed in U.S. Pat. No. 7,326,708, the contents of which are hereby incorporated by reference in their entirety. In another embodiment, the pharmaceutically acceptable salt and hydrate of sitagliptin is sitagliptin dihydrogen phosphate monohydrate salt. In another embodiment, the pharmaceutically acceptable salt and hydrate of sitagliptin is the crystalline sitagliptin dihydrogen phosphate monohydrate.

In one embodiment, the antioxidant is selected from the group: ascorbic acid, butylated hydroxytoluene, methyl gallate, ethyl gallate, propyl gallate, butyl gallate, pentyl gallate, octyl gallate, dodecyl (lauryl) gallate, butylated hydroxyanisole, thymol, tocopherol, and vitamin E. In another embodiment, the antioxidant is selected from the group: ascorbic acid, butylated hydroxytoluene, propyl gallate, butyl gallate, butylated hydroxyanisole, thymol, tocopherol, and vitamin E. In another embodiment, the antioxidant is selected from: propyl gallate and butylated hydroxyanisole. In another embodiment, the antioxidant is propyl gallate. In another embodiment, the antioxidant is butylated hydroxyanisole.

A composition comprising:
(a) about 99% by weight of a sitagliptin, or a pharmaceutically acceptable salt and/or hydrate thereof; and
(b) about 1% by weight of an antioxidant.

In one embodiment, the pharmaceutically acceptable salt of sitagliptin is the dihydrogen phosphate salt of structural formula I above (sitagliptin phosphate). In another embodiment, the pharmaceutically acceptable hydrate of sitagliptin is the monohydrate. In another embodiment the pharmaceutically acceptable salt and hydrate of sitagliptin is the dihydrogen phosphate monohydrate. In another embodiment, the form of the dihydrogen phosphate salt is the crystalline monohydrate disclosed in U.S. Pat. No. 7,326,708, the contents of which are hereby incorporated by reference in their entirety. In another embodiment, the pharmaceutically acceptable salt and hydrate of sitagliptin is sitagliptin dihydrogen phosphate monohydrate salt. In another embodiment, the pharmaceutically acceptable salt and hydrate of sitagliptin is the crystalline sitagliptin dihydrogen phosphate monohydrate.

In one embodiment, the antioxidant is selected from the group: ascorbic acid, butylated hydroxytoluene, methyl gallate, ethyl gallate, propyl gallate, butyl gallate, pentyl gallate, octyl gallate, dodecyl (lauryl) gallate, butylated hydroxyanisole, thymol, tocopherol, and vitamin E. In another embodiment, the antioxidant is selected from the group: ascorbic acid, butylated hydroxytoluene, propyl gallate, butyl gallate, butylated hydroxyanisole, thymol, tocopherol, and vitamin E. In another embodiment, the antioxidant is selected from: propyl gallate and butylated hydroxyanisole. In another embodiment, the antioxidant is propyl gallate. In another embodiment, the antioxidant is butylated hydroxyanisole.

A composition consisting essentially of
(a) about 99% by weight of a sitagliptin, or a pharmaceutically acceptable salt and/or hydrate thereof; and
(b) about 1% by weight of an antioxidant.

In one embodiment, the pharmaceutically acceptable salt of sitagliptin is the dihydrogen phosphate salt of structural formula I above (sitagliptin phosphate). In another embodiment, the pharmaceutically acceptable hydrate of sitagliptin is the monohydrate. In another embodiment the pharmaceutically acceptable salt and hydrate of sitagliptin is the dihydrogen phosphate monohydrate. In another embodiment, the form of the dihydrogen phosphate salt is the crystalline monohydrate disclosed in U.S. Pat. No. 7,326,708, the contents of which are hereby incorporated by reference in their entirety. In another embodiment, the pharmaceutically acceptable salt and hydrate of sitagliptin is sitagliptin dihydrogen phosphate monohydrate salt. In another embodiment, the pharmaceutically acceptable salt and hydrate of sitagliptin is the crystalline sitagliptin dihydrogen phosphate monohydrate.

In one embodiment, the antioxidant is selected from the group: ascorbic acid, butylated hydroxytoluene, methyl gallate, ethyl gallate, propyl gallate, butyl gallate, pentyl gallate, octyl gallate, dodecyl (lauryl) gallate, butylated hydroxyanisole, thymol, tocopherol, and vitamin E. In another embodiment, the antioxidant is selected from the group: ascorbic acid, butylated hydroxytoluene, propyl gallate, butyl gallate, butylated hydroxyanisole, thymol, tocopherol, and vitamin E. In another embodiment, the antioxidant is selected from: propyl gallate and butylated hydroxyanisole. In another embodiment, the antioxidant is propyl gallate. In another embodiment, the antioxidant is butylated hydroxyanisole.

A composition comprising:
(a) about 99.2% by weight of a sitagliptin, or a pharmaceutically acceptable salt and/or hydrate thereof; and
(b) about 0.8% by weight of an antioxidant.

In one embodiment, the pharmaceutically acceptable salt of sitagliptin is the dihydrogen phosphate salt of structural formula I above (sitagliptin phosphate). In another embodiment, the pharmaceutically acceptable hydrate of sitagliptin is the monohydrate. In another embodiment the pharmaceutically acceptable salt and hydrate of sitagliptin is the dihydrogen phosphate monohydrate. In another embodiment, the form of the dihydrogen phosphate salt is the crystalline monohydrate disclosed in U.S. Pat. No. 7,326,708, the contents of which are hereby incorporated by reference in their entirety. In another embodiment, the pharmaceutically acceptable salt and hydrate of sitagliptin is sitagliptin dihydrogen phosphate monohydrate salt. In another embodiment, the pharmaceutically acceptable salt and hydrate of sitagliptin is the crystalline sitagliptin dihydrogen phosphate monohydrate.

In one embodiment, the antioxidant is selected from the group: ascorbic acid, butylated hydroxytoluene, methyl gallate, ethyl gallate, propyl gallate, butyl gallate, pentyl gallate, octyl gallate, dodecyl (lauryl) gallate, butylated hydroxyanisole, thymol, tocopherol, and vitamin E. In another embodiment, the antioxidant is selected from the group: ascorbic acid, butylated hydroxytoluene, propyl gallate, butyl gallate, butylated hydroxyanisole, thymol, tocopherol, and vitamin E. In another embodiment, the antioxidant is selected from: propyl gallate and butylated hydroxyanisole. In another embodiment, the antioxidant is propyl gallate. In another embodiment, the antioxidant is butylated hydroxyanisole.

A composition consisting essentially of
(a) about 99.2% by weight of a sitagliptin, or a pharmaceutically acceptable salt and/or hydrate thereof; and
(b) about 0.8% by weight of an antioxidant.

In one embodiment, the pharmaceutically acceptable salt of sitagliptin is the dihydrogen phosphate salt of structural formula I above (sitagliptin phosphate). In another embodiment, the pharmaceutically acceptable hydrate of sitagliptin is the monohydrate. In another embodiment the pharmaceutically acceptable salt and hydrate of sitagliptin is the dihydrogen phosphate monohydrate. In another embodiment, the form of the dihydrogen phosphate salt is the crystalline monohydrate disclosed in U.S. Pat. No. 7,326,708, the contents of which are hereby incorporated by reference in their entirety. In another embodiment, the pharmaceutically acceptable salt and hydrate of sitagliptin is sitagliptin dihydrogen phosphate monohydrate salt. In another embodiment, the pharmaceutically acceptable salt and hydrate of sitagliptin is the crystalline sitagliptin dihydrogen phosphate monohydrate.

In one embodiment, the antioxidant is selected from the group: ascorbic acid, butylated hydroxytoluene, methyl gallate, ethyl gallate, propyl gallate, butyl gallate, pentyl gallate, octyl gallate, dodecyl (lauryl) gallate, butylated hydroxyanisole, thymol, tocopherol, and vitamin E. In another embodiment, the antioxidant is selected from the group: ascorbic acid, butylated hydroxytoluene, propyl gallate, butyl gallate, butylated hydroxyanisole, thymol, tocopherol, and vitamin E. In another embodiment, the antioxidant is selected from: propyl gallate and butylated hydroxyanisole. In another embodiment, the antioxidant is propyl gallate. In another embodiment, the antioxidant is butylated hydroxyanisole.

A composition comprising:
(a) about 99.5% by weight of a sitagliptin, or a pharmaceutically acceptable salt and/or hydrate thereof; and
(b) about 0.5% by weight of an antioxidant.

In one embodiment, the pharmaceutically acceptable salt of sitagliptin is the dihydrogen phosphate salt of structural formula I above (sitagliptin phosphate). In another embodiment, the pharmaceutically acceptable hydrate of sitagliptin is the monohydrate. In another embodiment the pharmaceutically acceptable salt and hydrate of sitagliptin is the dihydrogen phosphate monohydrate. In another embodiment, the form of the dihydrogen phosphate salt is the crystalline monohydrate disclosed in U.S. Pat. No. 7,326,708, the contents of which are hereby incorporated by reference in their entirety. In another embodiment, the pharmaceutically acceptable salt and hydrate of sitagliptin is sitagliptin dihydrogen phosphate monohydrate salt. In another embodiment, the pharmaceutically acceptable salt and hydrate of sitagliptin is the crystalline sitagliptin dihydrogen phosphate monohydrate.

In one embodiment, the antioxidant is selected from the group: ascorbic acid, butylated hydroxytoluene, methyl gallate, ethyl gallate, propyl gallate, butyl gallate, pentyl gallate, octyl gallate, dodecyl (lauryl) gallate, butylated hydroxyanisole, thymol, tocopherol, and vitamin E. In another embodiment, the antioxidant is selected from the group: ascorbic acid, butylated hydroxytoluene, propyl gallate, butyl gallate, butylated hydroxyanisole, thymol, tocopherol, and vitamin E. In another embodiment, the antioxidant is selected from: propyl gallate and butylated hydroxyanisole. In another embodiment, the antioxidant is propyl gallate. In another embodiment, the antioxidant is butylated hydroxyanisole.

A composition consisting essentially of
(a) about 99.5% by weight of a sitagliptin, or a pharmaceutically acceptable salt and/or hydrate thereof; and
(b) about 0.5% by weight of an antioxidant.

In one embodiment, the pharmaceutically acceptable salt of sitagliptin is the dihydrogen phosphate salt of structural formula I above (sitagliptin phosphate). In another embodiment, the pharmaceutically acceptable hydrate of sitagliptin is the monohydrate. In another embodiment the pharmaceutically acceptable salt and hydrate of sitagliptin is the dihydrogen phosphate monohydrate. In another embodiment, the form of the dihydrogen phosphate salt is the crystalline monohydrate disclosed in U.S. Pat. No. 7,326,708, the contents of which are hereby incorporated by reference in their entirety. In another embodiment, the pharmaceutically acceptable salt and hydrate of sitagliptin is sitagliptin dihydrogen phosphate monohydrate salt. In another embodiment, the pharmaceutically acceptable salt and hydrate of sitagliptin is the crystalline sitagliptin dihydrogen phosphate monohydrate.

In one embodiment, the antioxidant is selected from the group: ascorbic acid, butylated hydroxytoluene, methyl gallate, ethyl gallate, propyl gallate, butyl gallate, pentyl gallate, octyl gallate, dodecyl (lauryl) gallate, butylated hydroxyanisole, thymol, tocopherol, and vitamin E. In another embodiment, the antioxidant is selected from the group: ascorbic acid, butylated hydroxytoluene, propyl gallate, butyl gallate, butylated hydroxyanisole, thymol, tocopherol, and vitamin E. In another embodiment, the antioxidant is selected from: propyl gallate and butylated hydroxyanisole. In another embodiment, the antioxidant is propyl gallate. In another embodiment, the antioxidant is butylated hydroxyanisole.

A composition comprising:
(a) about 99.65% by weight of a sitagliptin, or a pharmaceutically acceptable salt and/or hydrate thereof; and
(b) about 0.35% by weight of an antioxidant.

In one embodiment, the pharmaceutically acceptable salt of sitagliptin is the dihydrogen phosphate salt of structural formula I above (sitagliptin phosphate). In another embodiment, the pharmaceutically acceptable hydrate of sitagliptin is the monohydrate. In another embodiment the pharmaceutically acceptable salt and hydrate of sitagliptin is the dihydrogen phosphate monohydrate. In another embodiment, the form of the dihydrogen phosphate salt is the crystalline monohydrate disclosed in U.S. Pat. No. 7,326,708, the contents of which are hereby incorporated by reference in their entirety. In another embodiment, the pharmaceutically acceptable salt and hydrate of sitagliptin is sitagliptin dihydrogen phosphate monohydrate salt. In another embodiment, the pharmaceutically acceptable salt and hydrate of sitagliptin is the crystalline sitagliptin dihydrogen phosphate monohydrate.

In one embodiment, the antioxidant is selected from the group: ascorbic acid, butylated hydroxytoluene, methyl gallate, ethyl gallate, propyl gallate, butyl gallate, pentyl gallate, octyl gallate, dodecyl (lauryl) gallate, butylated hydroxyanisole, thymol, tocopherol, and vitamin E. In another embodiment, the antioxidant is selected from the group: ascorbic acid, butylated hydroxytoluene, propyl gallate, butyl gallate, butylated hydroxyanisole, thymol, tocopherol, and vitamin E. In another embodiment, the antioxidant is selected from: propyl gallate and butylated hydroxyanisole. In another embodiment, the antioxidant is propyl gallate. In another embodiment, the antioxidant is butylated hydroxyanisole.

A composition consisting essentially of
(a) about 99.65% by weight of a sitagliptin, or a pharmaceutically acceptable salt and/or hydrate thereof; and
(b) about 0.35% by weight of an antioxidant.

In one embodiment, the pharmaceutically acceptable salt of sitagliptin is the dihydrogen phosphate salt of structural formula I above (sitagliptin phosphate). In another embodiment, the pharmaceutically acceptable hydrate of sitagliptin is the monohydrate. In another embodiment the pharmaceutically acceptable salt and hydrate of sitagliptin is the dihydrogen phosphate monohydrate. In another embodiment, the form of the dihydrogen phosphate salt is the crystalline monohydrate disclosed in U.S. Pat. No. 7,326,708, the contents of which are hereby incorporated by reference in their entirety. In another embodiment, the pharmaceutically acceptable salt and hydrate of sitagliptin is sitagliptin dihydrogen phosphate monohydrate salt. In another embodiment, the pharmaceutically acceptable salt and hydrate of sitagliptin is the crystalline sitagliptin dihydrogen phosphate monohydrate.

In one embodiment, the antioxidant is selected from the group: ascorbic acid, butylated hydroxytoluene, methyl gallate, ethyl gallate, propyl gallate, butyl gallate, pentyl gallate, octyl gallate, dodecyl (lauryl) gallate, butylated hydroxyanisole, thymol, tocopherol, and vitamin E. In another embodiment, the antioxidant is selected from the group: ascorbic acid, butylated hydroxytoluene, propyl gallate, butyl gallate, butylated hydroxyanisole, thymol, tocopherol, and vitamin E. In another embodiment, the antioxidant is selected from: propyl gallate and butylated hydroxyanisole. In another embodiment, the antioxidant is propyl gallate. In another embodiment, the antioxidant is butylated hydroxyanisole.

A composition comprising:
(a) about 99.70% by weight of a sitagliptin, or a pharmaceutically acceptable salt and/or hydrate thereof; and
(b) about 0.30% by weight of an antioxidant.

In one embodiment, the pharmaceutically acceptable salt of sitagliptin is the dihydrogen phosphate salt of structural formula I above (sitagliptin phosphate). In another embodiment, the pharmaceutically acceptable hydrate of sitagliptin is the monohydrate. In another embodiment the pharmaceutically acceptable salt and hydrate of sitagliptin is the dihydrogen phosphate monohydrate. In another embodiment, the form of the dihydrogen phosphate salt is the crystalline monohydrate disclosed in U.S. Pat. No. 7,326,708, the contents of which are hereby incorporated by reference in their entirety. In another embodiment, the pharmaceutically acceptable salt and hydrate of sitagliptin is sitagliptin dihydrogen phosphate monohydrate salt. In another embodiment, the pharmaceutically acceptable salt and hydrate of sitagliptin is the crystalline sitagliptin dihydrogen phosphate monohydrate.

In one embodiment, the antioxidant is selected from the group: ascorbic acid, butylated hydroxytoluene, methyl gallate, ethyl gallate, propyl gallate, butyl gallate, pentyl gallate, octyl gallate, dodecyl (lauryl) gallate, butylated hydroxyanisole, thymol, tocopherol, and vitamin E. In another embodiment, the antioxidant is selected from the group: ascorbic acid, butylated hydroxytoluene, propyl gallate, butyl gallate, butylated hydroxyanisole, thymol, tocopherol, and vitamin E. In another embodiment, the antioxidant is selected from: propyl gallate and butylated hydroxyanisole. In another embodiment, the antioxidant is propyl gallate. In another embodiment, the antioxidant is butylated hydroxyanisole.

A composition consisting essentially of
(a) about 99.70% by weight of a sitagliptin, or a pharmaceutically acceptable salt and/or hydrate thereof; and
(b) about 0.30% by weight of an antioxidant.

In one embodiment, the pharmaceutically acceptable salt of sitagliptin is the dihydrogen phosphate salt of structural formula I above (sitagliptin phosphate). In another embodiment, the pharmaceutically acceptable hydrate of sitagliptin is the monohydrate. In another embodiment the pharmaceutically acceptable salt and hydrate of sitagliptin is the dihydrogen phosphate monohydrate. In another embodiment, the form of the dihydrogen phosphate salt is the crystalline monohydrate disclosed in U.S. Pat. No. 7,326,708, the contents of which are hereby incorporated by reference in their entirety. In another embodiment, the pharmaceutically acceptable salt and hydrate of sitagliptin is sitagliptin dihydrogen phosphate monohydrate salt. In another embodiment, the pharmaceutically acceptable salt and hydrate of sitagliptin is the crystalline sitagliptin dihydrogen phosphate monohydrate.

In one embodiment, the antioxidant is selected from the group: ascorbic acid, butylated hydroxytoluene, methyl gallate, ethyl gallate, propyl gallate, butyl gallate, pentyl gallate, octyl gallate, dodecyl (lauryl) gallate, butylated hydroxyanisole, thymol, tocopherol, and vitamin E. In another embodiment, the antioxidant is selected from the group: ascorbic acid, butylated hydroxytoluene, propyl gallate, butyl gallate, butylated hydroxyanisole, thymol, tocopherol, and vitamin E. In another embodiment, the antioxidant is selected from: propyl gallate and butylated hydroxyanisole. In another embodiment, the antioxidant is propyl gallate. In another embodiment, the antioxidant is butylated hydroxyanisole.

A composition comprising:
(a) about 99.85% by weight of a sitagliptin, or a pharmaceutically acceptable salt and/or hydrate thereof; and
(b) about 0.15% by weight of an antioxidant.

In one embodiment, the pharmaceutically acceptable salt of sitagliptin is the dihydrogen phosphate salt of structural formula I above (sitagliptin phosphate). In another embodiment, the pharmaceutically acceptable hydrate of sitagliptin is the monohydrate. In another embodiment the pharmaceutically acceptable salt and hydrate of sitagliptin is the dihydrogen phosphate monohydrate. In another embodiment, the form of the dihydrogen phosphate salt is the crystalline monohydrate disclosed in U.S. Pat. No. 7,326,708, the contents of which are hereby incorporated by reference in their entirety. In another embodiment, the pharmaceutically acceptable salt and hydrate of sitagliptin is sitagliptin dihydrogen phosphate monohydrate salt. In another embodiment, the pharmaceutically acceptable salt and hydrate of sitagliptin is the crystalline sitagliptin dihydrogen phosphate monohydrate.

In one embodiment, the antioxidant is selected from the group: ascorbic acid, butylated hydroxytoluene, methyl gallate, ethyl gallate, propyl gallate, butyl gallate, pentyl gallate, octyl gallate, dodecyl (lauryl) gallate, butylated hydroxyanisole, thymol, tocopherol, and vitamin E. In another embodiment, the antioxidant is selected from the group: ascorbic acid, butylated hydroxytoluene, propyl gallate, butyl gallate, butylated hydroxyanisole, thymol, tocopherol, and vitamin E. In another embodiment, the antioxidant is selected from: propyl gallate and butylated hydroxyanisole. In another embodiment, the antioxidant is propyl gallate. In another embodiment, the antioxidant is butylated hydroxyanisole.

A composition consisting essentially of
(a) about 99.85% by weight of a sitagliptin, or a pharmaceutically acceptable salt and/or hydrate thereof; and
(b) about 0.15% by weight of an antioxidant.

In one embodiment, the pharmaceutically acceptable salt of sitagliptin is the dihydrogen phosphate salt of structural formula I above (sitagliptin phosphate). In another embodiment, the pharmaceutically acceptable hydrate of sitagliptin is the monohydrate. In another embodiment the pharmaceutically acceptable salt and hydrate of sitagliptin is the dihydrogen phosphate monohydrate. In another embodiment, the form of the dihydrogen phosphate salt is the crystalline monohydrate disclosed in U.S. Pat. No. 7,326,708, the contents of which are hereby incorporated by reference in their entirety. In another embodiment, the pharmaceutically acceptable salt and hydrate of sitagliptin is sitagliptin dihydrogen phosphate monohydrate salt. In another embodiment, the pharmaceutically acceptable salt and hydrate of sitagliptin is the crystalline sitagliptin dihydrogen phosphate monohydrate.

In one embodiment, the antioxidant is selected from the group: ascorbic acid, butylated hydroxytoluene, methyl gallate, ethyl gallate, propyl gallate, butyl gallate, pentyl gallate, octyl gallate, dodecyl (lauryl) gallate, butylated hydroxyanisole, thymol, tocopherol, and vitamin E. In another embodiment, the antioxidant is selected from the group: ascorbic acid, butylated hydroxytoluene, propyl gallate, butyl gallate, butylated hydroxyanisole, thymol, tocopherol, and vitamin E. In another embodiment, the antioxidant is selected from: propyl gallate and butylated hydroxyanisole. In another embodiment, the antioxidant is propyl gallate. In another embodiment, the antioxidant is butylated hydroxyanisole.

A composition comprising:
(a) about 99.88% by weight of a sitagliptin, or a pharmaceutically acceptable salt and/or hydrate thereof; and
(b) about 0.12% by weight of an antioxidant.

In one embodiment, the pharmaceutically acceptable salt of sitagliptin is the dihydrogen phosphate salt of structural formula I above (sitagliptin phosphate). In another embodiment, the pharmaceutically acceptable hydrate of sitagliptin is the monohydrate. In another embodiment the pharmaceutically acceptable salt and hydrate of sitagliptin is the dihydrogen phosphate monohydrate. In another embodiment, the form of the dihydrogen phosphate salt is the crystalline monohydrate disclosed in U.S. Pat. No. 7,326,708, the contents of which are hereby incorporated by reference in their entirety. In another embodiment, the pharmaceutically acceptable salt and hydrate of sitagliptin is sitagliptin dihydrogen phosphate monohydrate salt. In another embodiment, the pharmaceutically acceptable salt and hydrate of sitagliptin is the crystalline sitagliptin dihydrogen phosphate monohydrate.

In one embodiment, the antioxidant is selected from the group: ascorbic acid, butylated hydroxytoluene, methyl gallate, ethyl gallate, propyl gallate, butyl gallate, pentyl gallate, octyl gallate, dodecyl (lauryl) gallate, butylated hydroxyanisole, thymol, tocopherol, and vitamin E. In another embodiment, the antioxidant is selected from the group: ascorbic acid, butylated hydroxytoluene, propyl gallate, butyl gallate, butylated hydroxyanisole, thymol, tocopherol, and vitamin E. In another embodiment, the antioxidant is selected from: propyl gallate and butylated hydroxyanisole. In another embodiment, the antioxidant is propyl gallate. In another embodiment, the antioxidant is butylated hydroxyanisole.

A composition consisting essentially of
(a) about 99.88% by weight of a sitagliptin, or a pharmaceutically acceptable salt and/or hydrate thereof; and
(b) about 0.12% by weight of an antioxidant.

In one embodiment, the pharmaceutically acceptable salt of sitagliptin is the dihydrogen phosphate salt of structural formula I above (sitagliptin phosphate). In another embodiment, the pharmaceutically acceptable hydrate of sitagliptin is the monohydrate. In another embodiment the pharmaceutically acceptable salt and hydrate of sitagliptin is the dihydrogen phosphate monohydrate. In another embodiment, the form of the dihydrogen phosphate salt is the crystalline monohydrate disclosed in U.S. Pat. No. 7,326,708, the contents of which are hereby incorporated by reference in their entirety. In another embodiment, the pharmaceutically acceptable salt and hydrate of sitagliptin is sitagliptin dihydrogen phosphate monohydrate salt. In another embodiment, the pharmaceutically acceptable salt and hydrate of sitagliptin is the crystalline sitagliptin dihydrogen phosphate monohydrate.

In one embodiment, the antioxidant is selected from the group: ascorbic acid, butylated hydroxytoluene, methyl gallate, ethyl gallate, propyl gallate, butyl gallate, pentyl gallate, octyl gallate, dodecyl (lauryl) gallate, butylated hydroxyanisole, thymol, tocopherol, and vitamin E. In another embodiment, the antioxidant is selected from the group: ascorbic acid, butylated hydroxytoluene, propyl gallate, butyl gallate, butylated hydroxyanisole, thymol, tocopherol, and vitamin E. In another embodiment, the antioxidant is selected from: propyl gallate and butylated hydroxyanisole. In another embodiment, the antioxidant is propyl gallate. In another embodiment, the antioxidant is butylated hydroxyanisole.

A composition comprising:
(a) about 99.9% by weight of a sitagliptin, or a pharmaceutically acceptable salt and/or hydrate thereof; and
(b) about 0.1% by weight of an antioxidant.

In one embodiment, the pharmaceutically acceptable salt of sitagliptin is the dihydrogen phosphate salt of structural formula I above (sitagliptin phosphate). In another embodiment, the pharmaceutically acceptable hydrate of sitagliptin is the monohydrate. In another embodiment the pharmaceutically acceptable salt and hydrate of sitagliptin is the dihydrogen phosphate monohydrate. In another embodiment, the form of the dihydrogen phosphate salt is the crystalline monohydrate disclosed in U.S. Pat. No. 7,326,708, the contents of which are hereby incorporated by reference in their entirety. In another embodiment, the pharmaceutically acceptable salt and hydrate of sitagliptin is sitagliptin dihydrogen phosphate monohydrate salt. In another embodiment, the pharmaceutically acceptable salt and hydrate of sitagliptin is the crystalline sitagliptin dihydrogen phosphate monohydrate.

In one embodiment, the antioxidant is selected from the group: ascorbic acid, butylated hydroxytoluene, methyl gallate, ethyl gallate, propyl gallate, butyl gallate, pentyl gallate, octyl gallate, dodecyl (lauryl) gallate, butylated hydroxyanisole, thymol, tocopherol, and vitamin E. In another embodiment, the antioxidant is selected from the group: ascorbic acid, butylated hydroxytoluene, propyl gallate, butyl gallate, butylated hydroxyanisole, thymol, tocopherol, and vitamin E. In another embodiment, the antioxidant is selected from: propyl gallate and butylated hydroxyanisole. In another embodiment, the antioxidant is propyl gallate. In another embodiment, the antioxidant is butylated hydroxyanisole.

A composition consisting essentially of
(a) about 99.9% by weight of a sitagliptin, or a pharmaceutically acceptable salt and/or hydrate thereof; and
(b) about 0.1% by weight of an antioxidant.

In one embodiment, the pharmaceutically acceptable salt of sitagliptin is the dihydrogen phosphate salt of structural formula I above (sitagliptin phosphate). In another embodiment, the pharmaceutically acceptable hydrate of sitagliptin is the monohydrate. In another embodiment the pharmaceutically acceptable salt and hydrate of sitagliptin is the dihydrogen phosphate monohydrate. In another embodiment, the form of the dihydrogen phosphate salt is the crystalline monohydrate disclosed in U.S. Pat. No. 7,326,708, the contents of which are hereby incorporated by reference in their entirety. In another embodiment, the pharmaceutically acceptable salt and hydrate of sitagliptin is sitagliptin dihydrogen phosphate monohydrate salt. In another embodiment, the pharmaceutically acceptable salt and hydrate of sitagliptin is the crystalline sitagliptin dihydrogen phosphate monohydrate.

In one embodiment, the antioxidant is selected from the group: ascorbic acid, butylated hydroxytoluene, methyl gallate, ethyl gallate, propyl gallate, butyl gallate, pentyl gallate, octyl gallate, dodecyl (lauryl) gallate, butylated hydroxyanisole, thymol, tocopherol, and vitamin E. In another embodiment, the antioxidant is selected from the group: ascorbic acid, butylated hydroxytoluene, propyl gallate, butyl gallate, butylated hydroxyanisole, thymol, tocopherol, and vitamin E. In another embodiment, the antioxidant is selected from: propyl gallate and butylated hydroxyanisole. In another embodiment, the antioxidant is propyl gallate. In another embodiment, the antioxidant is butylated hydroxyanisole.

The preparation of sitagliptin, and pharmaceutically acceptable salts thereof, is disclosed in U.S. Pat. No. 6,699,871, the contents of which are herein incorporated by reference in their entirety. The preparation of sitagliptin phosphate monohydrate is disclosed in U.S. Pat. No. 7,326,708, the contents of which are hereby incorporated by reference in their entirety.

Also provided are processes to prepare the formulation of sitagliptin dihydrogen phosphate monohydrate and an antioxidant.

Provided is a process of preparing a sitagliptin and antioxidant complex or composition comprising the steps of
1) dissolving sitagliptin dihydrogen phosphate monohydrate in a suitable solvent at suitable temperature to provide a solution;
2) adding sitagliptin dihydrogen phosphate monohydrate seed to the solution of Step 1; 3) adding anti-solvent to the solution of Step 2 to form a mixture;
4) adding an antioxidant to the mixture of Step 3 to form a mixture;
5) cooling the mixture of Step 4; and
6) filtering the mixture of Step 5 to isolate the solid sitagliptin dihydrogen phosphate monohydrate-antioxidant complex or composition.

In one embodiment, the suitable solvent in Step 1 is a mixture of water and isopropanol, and the suitable temperature in Step 1 is about 65 °C to 80 °C.

In another embodiment, the suitable solvent in Step 1 is a mixture of a 79:21 of isopropanol:water, and the suitable temperature in Step 1 is about 70 °C to 80 °C.

In another embodiment, the process further comprises cooling the solution of Step 1 to about 65 °C to 67 °C. In a class of this embodiment, the solution of Step 1 is cooled before addition of the sitagliptin dihydrogen phosphate monohydrate seed.

In another embodiment, the sitagliptin dihydrogen phosphate monohydrate seed of Step 2 is milled.

In another embodiment, the process further comprises cooling the solution of Step 2 to about 15 °C to 25 °C.

In another embodiment, the anti-solvent of Step 3 is isopropanol.

In another embodiment, the mixture of Step 5 is cooled to about −10 to 0 °C.

In another embodiment, the process further comprises drying the solid sitagliptin dihydrogen phosphate monohydrate-antioxidant complex or composition of Step 6.

In another embodiment, the process further comprises de-lumping and blending the dried sitagliptin dihydrogen phosphate monohydrate-antioxidant complex or composition of Step 6.

In another embodiment, the process further comprises washing the wet sitagliptin dihydrogen phosphate monohydrate antioxidant complex or composition with a cold wash solution comprising propyl gallate in aqueous isopropanol after filtering in Step 6.

In another embodiment, the antioxidant of Step 4 is propyl gallate; and the sitagliptin dihydrogen phosphate monohydrate antioxidant complex or composition of Step 6 is the sitagliptin dihydrogen phosphate monohydrate propyl gallate complex or composition.

In another embodiment, the antioxidant of Step 4 is butylated hydroxyanisole; and the sitagliptin dihydrogen phosphate monohydrate antioxidant complex or composition of Step 6 is the sitagliptin dihydrogen phosphate monohydrate butylated hydroxyanisole complex or composition.

In another embodiment, the dried solid sitagliptin dihydrogen phosphate monohydrate-antioxidant complex or composition of Step 6 is crystalline.

Also provided is a process of preparing a sitagliptin and antioxidant complex or composition comprising the steps of:
1) dissolving sitagliptin dihydrogen phosphate monohydrate in a suitable solvent at suitable temperature to provide a solution;
2) adding sitagliptin dihydrogen phosphate monohydrate seed to the solution of Step 2;
3) cooling the solution of Step 2;
4) adding anti-solvent to the solution of Step 4 to form a mixture;
5) adding an antioxidant to the mixture of Step 5 to form a mixture;
6) cooling the mixture of Step 5; and
7) filtering the mixture of Step 6 to isolate the solid sitagliptin dihydrogen phosphate monohydrate-antioxidant complex or composition.

In one embodiment, the suitable solvent in Step 1 is a mixture of water and isopropanol, and the suitable temperature in Step 1 is about 65 °C to 80 °C.

In another embodiment, the suitable solvent in Step 1 is a mixture of a 79:21 of isopropanol:water, and the suitable temperature in Step 1 is about 70 °C to 80 °C.

In another embodiment, the process further comprises cooling the solution of Step 1 to about 65 °C to 67 °C.

In another embodiment, the process further comprises cooling the solution of Step 2 to about 15 °C to 25 °C.

In another embodiment, the sitagliptin dihydrogen phosphate monohydrate seed of Step 2 is milled.

In another embodiment, the anti-solvent of Step 4 is isopropanol.

In another embodiment, the mixture of Step 5 is cooled to about −10 to 0 °C.

In another embodiment, the process further comprises drying the solid sitagliptin dihydrogen phosphate monohydrate-antioxidant complex or composition of Step 7.

In another embodiment, the process further comprises de-lumping and blending the dried sitagliptin dihydrogen phosphate monohydrate-antioxidant complex or composition of Step 7.

In another embodiment, the process further comprises washing the wet sitagliptin dihydrogen phosphate monohydrate antioxidant complex or composition with a cold wash solution comprising propyl gallate in aqueous isopropanol after filtering in Step 7.

In another embodiment, the antioxidant of Step 5 is propyl gallate; and the sitagliptin dihydrogen phosphate monohydrate antioxidant complex or composition of Step 7 is the sitagliptin dihydrogen phosphate monohydrate propyl gallate complex or composition.

In another embodiment, the antioxidant of Step 5 is butylated hydroxyanisole; and the sitagliptin dihydrogen phosphate monohydrate antioxidant complex or composition of Step 7 is the sitagliptin dihydrogen phosphate monohydrate butylated hydroxyanisole complex or composition.

In another embodiment, the dried solid sitagliptin dihydrogen phosphate monohydrate-antioxidant complex or composition of Step 7 is crystalline.

Also provided is a process of preparing a sitagliptin and antioxidant complex or composition comprising the steps of
1) dissolving sitagliptin dihydrogen phosphate monohydrate in a suitable solvent at suitable temperature to provide a solution;
2) cooling the solution of Step 1;
3) adding sitagliptin dihydrogen phosphate monohydrate seed to the solution of Step 2;
4) cooling the solution of Step 3;
5) adding anti-solvent to the solution of Step 4 to form a mixture;
6) adding an antioxidant to the mixture of Step 5 to form a mixture;
7) cooling the mixture of Step 6; and
8) filtering the mixture of Step 7 to isolate the solid sitagliptin dihydrogen phosphate monohydrate-antioxidant complex or composition.

In one embodiment, the suitable solvent in Step 1 is a mixture of water and isopropanol, and the suitable temperature in Step 1 is about 65 °C to 80 °C.

In another embodiment, the suitable solvent in Step 1 is a mixture of a 79:21 of isopropanol:water, and the suitable temperature in Step 1 is about 70 °C to 80 °C.

In another embodiment, the process further comprises cooling the solution of Step 1 to about 65 °C to 67 °C.

In another embodiment, the sitagliptin dihydrogen phosphate monohydrate seed of Step 3 is milled.

In another embodiment, the process further comprises cooling the solution of Step 3 to about 15 °C to 25 °C.

In another embodiment, the anti-solvent of Step 5 is isopropanol.

In another embodiment, the mixture of Step 6 is cooled to about −10 to 0 °C.

In another embodiment, the process further comprises drying the solid sitagliptin dihydrogen phosphate monohydrate-antioxidant complex or composition of Step 8.

In another embodiment, the process further comprises de-lumping and blending the dried sitagliptin dihydrogen phosphate monohydrate-antioxidant complex or composition of Step 8.

In another embodiment, the process further comprises washing the wet sitagliptin dihydrogen phosphate monohydrate antioxidant complex or composition with a cold wash solution comprising propyl gallate in aqueous isopropanol after filtering in Step 8.

In another embodiment, the antioxidant of Step 6 is propyl gallate; and the sitagliptin dihydrogen phosphate monohydrate antioxidant complex or composition of Step 8 is the sitagliptin dihydrogen phosphate monohydrate propyl gallate complex or composition.

In another embodiment, the antioxidant of Step 6 is butylated hydroxyanisole; and the sitagliptin dihydrogen phosphate monohydrate antioxidant complex or composition of Step 8 is the sitagliptin dihydrogen phosphate monohydrate butylated hydroxyanisole complex or composition.

In another embodiment, the dried solid sitagliptin dihydrogen phosphate monohydrate-antioxidant complex or composition of Step 8 is crystalline.

Additionally provided is a process of preparing a sitagliptin and antioxidant complex or composition comprising the steps of
1) dissolving sitagliptin dihydrogen phosphate monohydrate in a suitable solvent at suitable temperature to provide a solution;
2) cooling the solution of Step 1 to the seed point temperature;
3) adding sitagliptin dihydrogen phosphate monohydrate milled seed to the solution of Step 2;
4) cooling the solution of Step 3 to about 15 °C to 25 °C:
5) adding anti-solvent to the solution of Step 4 to form a mixture;
6) adding an antioxidant to the mixture of Step 5 to form a mixture;
7) cooling the mixture of Step 6 to about −10 to 0 °C;
8) filtering the mixture of Step 7 to isolate the solid sitagliptin dihydrogen phosphate monohydrate-antioxidant composition; and
9) drying the solid sitagliptin dihydrogen phosphate monohydrate-antioxidant composition.

In one embodiment, the process further comprises de-lumping and blending the sitagliptin dihydrogen phosphate monohydrate-antioxidant composition of Step 9.

In another embodiment, the process further comprises washing the wet sitagliptin dihydrogen phosphate monohydrate antioxidant composition with a cold wash solution comprising propyl gallate in aqueous isopropanol after filtering in Step 8.

In another embodiment, the suitable solvent in Step 1 is a mixture of water and isopropanol, and the suitable temperature in Step 1 is about 65 °C to 80 °C.

In another embodiment, the suitable solvent in Step 1 is a mixture of a 79:21 of isopropanol:water, and the suitable temperature in Step 1 is about 70 °C to 80 °C.

In another embodiment, the temperature in Step 2 is about 65 °C to 67 °C.

In another embodiment, the antisolvent of Step 4 is isopropanol.

In another embodiment, the antioxidant of Step 6 is propyl gallate; and the sitagliptin dihydrogen phosphate monohydrate antioxidant composition of Step 9 is the sitagliptin dihydrogen phosphate monohydrate propyl gallate composition.

In another embodiment, the antioxidant of Step 6 is butylated hydroxyanisole; and the sitagliptin dihydrogen phosphate monohydrate antioxidant composition of Step 9 is the sitagliptin dihydrogen phosphate monohydrate butylated hydroxyanisole composition.

In another embodiment, the solid sitagliptin dihydrogen phosphate monohydrate-antioxidant composition of Steps 8 and 9 is crystalline.

Additionally provided are methods for the treatment of Type 2 diabetes by administering to a host in need of such treatment a pharmaceutical composition comprising the sitagliptin dihydrogen phosphate monohydrate antioxidant complex or composition. In one embodiment, the pharmaceutical composition contains a therapeutically effective amount of sitagliptin dihydrogen phosphate monohydrate in the sitagliptin dihydrogen phosphate monohydrate antioxidant complex or composition. In another embodiment, the antioxidant is propyl gallate. In another embodiment, the antioxidant is butylated hydroxyanisole.

These and other aspects of the disclosure will become readily apparent from the detailed description which follows.

The following schemes, procedures, processes and examples further describe and demonstrate embodiments within the scope of the present disclosure. The examples are given solely for the purpose of illustration and are not intended to be construed as limitations as many variations thereof are possible without departing from the spirit and scope of the present disclosure.

Abbreviations: ≤ is less than or equal to; BHA is butylated hydroxyanisole; DI is deionized water; dibasic calcium phosphate is equivalent to dicalcium phosphate and dibasic calcium phosphate anhydrous: hr is hour(s); IPA is isopropanol or 2-propanol; MPa is megapascal; ng is nanogram; NTTP is 7-nitroso-3-(trifluoromethyl)-5,6,7,8-tetrahydro-[1,2,4]triazolo[4,3-a]pyrazine; PG is propyl gallate; ppm is parts per million; RH is relative humidity; and w/v is weight by weight or weight for weight.

37 ng of NTTP in a tablet comprising 100 mg of sitagliptin free base is equivalent to 0.37 ppm NTTP. 18.5 ng of NTTP in a tablet comprising 50 mg of sitagliptin free base is equivalent to 0.37 ppm NTTP. 9.25 ng of NTTP in a tablet comprising 25 mg of sitagliptin free base is equivalent to 0.37 ppm NTTP. 4.625 ng of NTTP in a tablet comprising 12.5 mg of sitagliptin free base is equivalent to 0.37 ppm NTTP.

The term “wt %”. “weight %” and “weight percent” means the weight of one component divided by the total weight of all of the components in the mixture or solution. For example, for a two-component system containing components A and B, the weight percent of component A is (A/(A+B))×100.

Sitagliptin antioxidant complex is equivalent to sitagliptin antioxidant composition.

Sitagliptin phosphate antioxidant complex is equivalent to sitagliptin phosphate antioxidant composition. Sitagliptin phosphate monohydrate antioxidant complex is equivalent to sitagliptin phosphate monohydrate antioxidant composition. Sitagliptin dihydrogen phosphate monohydrate antioxidant complex is equivalent to sitagliptin dihydrogen phosphate monohydrate antioxidant composition.

Sitagliptin propyl gallate complex is equivalent to sitagliptin propyl gallate composition. Sitagliptin phosphate propyl gallate complex is equivalent to sitagliptin phosphate propyl gallate composition. Sitagliptin phosphate monohydrate propyl gallate complex is equivalent to sitagliptin phosphate monohydrate propyl gallate composition.

Sitagliptin dihydrogen phosphate monohydrate propyl gallate complex is equivalent to sitagliptin dihydrogen phosphate monohydrate propyl gallate composition.

Sitagliptin butylated hydroxyanisole complex is equivalent to sitagliptin butylated hydroxyanisole composition. Sitagliptin phosphate butylated hydroxyanisole complex is equivalent to sitagliptin phosphate butylated hydroxyanisole composition. Sitagliptin phosphate monohydrate butylated hydroxyanisole complex is equivalent to sitagliptin phosphate monohydrate butylated hydroxvanisole composition. Sitagliptin dihydrogen phosphate monohydrate butylated hydroxyanisole complex is equivalent to sitagliptin dihydrogen phosphate monohydrate butylated hydroxyanisole composition.

In the sitagliptin antioxidant complex or composition, the sitagliptin phosphate antioxidant complex or composition, the sitagliptin phosphate monohydrate antioxidant complex or composition, and the sitagliptin dihydrogen phosphate monohydrate antioxidant complex or composition, the antioxidant, including propyl gallate and butylated hydroxyanisole, is deposited on, or associated with, sitagliptin before and/or after drying. In one embodiment, the antioxidant, including propyl gallate and butylated hydroxyanisole, is deposited on the sitagliptin dihydrogen phosphate monohydrate solid before and/or after drying. In another embodiment, the antioxidant, including propyl gallate and butylated hydroxyanisole, is deposited on the sitagliptin dihydrogen phosphate monohydrate solid before and/or after drying. In another embodiment, the antioxidant, including propyl gallate and butylated hydroxyanisole, is deposited on sitagliptin dihydrogen phosphate monohydrate crystals before and/or after drying. In another embodiment, the antioxidant, including propyl gallate and butylated hydroxyanisole, is deposited on the dried sitagliptin dihydrogen phosphate monohydrate crystals before and/or after drying. In another embodiment, the antioxidant, including propyl gallate and butylated hydroxyanisole, is a solid deposited on sitagliptin dihydrogen phosphate monohydrate. In another embodiment, the antioxidant, including propyl gallate and butylated hydroxyanisole, is a glass deposited on sitagliptin dihydrogen phosphate monohydrate.

“Core tablet” refers to a pharmaceutical composition that is formed or compressed into a tablet without film coating.

“Core formulation” refers to a pharmaceutical composition that is not formed or compressed into a tablet, and that is not film coated.

“JANUVIA® core formulation” refers to a pharmaceutical composition comprising sitagliptin dihydrogen phosphate monohydrate, microcrystalline cellulose, anhydrous dibasic calcium phosphate, croscarmellose sodium, magnesium stearate, and sodium stearyl fumarate. “JANUVIA® core tablet” is a compressed or formed tablet comprising sitagliptin dihydrogen phosphate monohydrate, microcrystalline cellulose, anhydrous dibasic calcium phosphate, croscarmellose sodium, magnesium stearate, and sodium stearyl fumarate without a film coating.

“JANUVIA® 100 mg core tablet” contains 128.5 mg of sitagliptin dihydrogen phosphate monohydrate, which is equivalent to 100 mg of the sitagliptin free base.

“JANUVIA®50 mg core tablet” contains 64.25 mg of sitagliptin dihydrogen phosphate monohydrate, which is equivalent to 50 mg of the sitagliptin free base.

“JANUVIA®) 25 mg core tablet” contains 32.13 mg of sitagliptin dihydrogen phosphate monohydrate, which is equivalent to 25 mg of the sitagliptin free base.

“STEGLUJAN® core formulation” is a pharmaceutical composition comprising sitagliptin dihydrogen phosphate monohydrate, ertugliflozin L-pyroglutamic acid, microcrystalline cellulose, dicalcium phosphate, croscarmellose sodium, magnesium stearate, and sodium stearyl fumarate, and optionally Opadry20 and/or camauba wax.

“STEGLUJAN® core tablet” is a compressed or formed tablet comprising sitagliptin dihydrogen phosphate monohydrate, ertugliflozin L-pyroglutamic acid, microcrystalline cellulose, dicalcium phosphate, croscarmellose sodium, magnesium stearate, and sodium stearyl fumarate, and optionally Opadry20 and/or carnauba wax, without a film coating.

“STEGLUJAN® 5 mg/100 mg core tablet” comprises 6.48 mg ertugliflozin L-pyroglutamic acid and 128.5 mg of sitagliptin phosphate monohydrate, which is equivalent to 5 mg of ertugliflozin and 100 mg of the sitagliptin free base.

“STEGLUJAN® 15 mg/100 mg core tablet” comprises 19.43 mg ertugliflozin L-pyroglutamic acid and 128.5 mg of sitagliptin phosphate monohydrate, which is equivalent to 15 mg of ertugliflozin and 100 mg of the sitagliptin free base.

### General Scheme

### Sitagliptin Dihydrogen Phosphate Monohydrate-Antioxidant Complex or Composition Crystallization Procedure

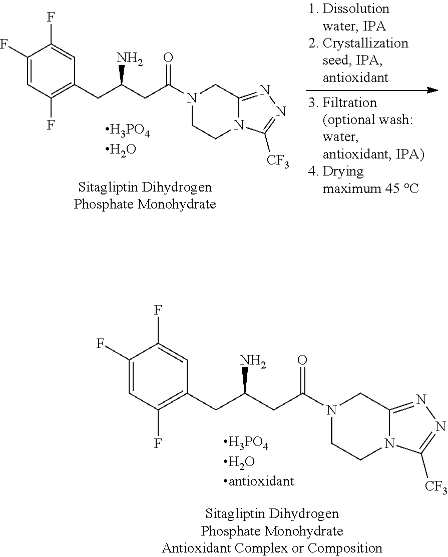 [SMILES: N[C@@H](CC(=O)N1CCn2c(nnc2C(F)(F)F)C1)Cc1cc(F)c(F)cc1F ; N[C@@H](CC(=O)N1CCn2c(nnc2C(F)(F)F)C1)Cc1cc(F)c(F)cc1F]

Sitagliptin dihydrogen phosphate monohydrate can be crystallized or recrystallized to form a complex or composition with an antioxidant as provided in the procedure below.

Procedure for the Dissolution of Sitagliptin Dihydrogen Phosphate Monohydrate (wherein X is the mass (or weight) of sitagliptin dihydrogen phosphate monohydrate):
1. Charge 0.920× (by mass) of deionized water to reactor 1;
2. Charge 2.247× (by mass) of isopropanol or 2-propanol to reactor 1;
3. Agitate and bring the contents of reactor 1 to about °15 °C to 25 °C;
4. Charge 1× of sitagliptin dihydrogen phosphate monohydrate to reactor 1;
5. Heat contents of reactor 1 to about 65 °C to 80 °C and age for a minimum of 1 hour until the solids are dissolved;
6. Transfer contents of reactor 1 to reactor 2 through a ≤1.0-micron filter, maintaining the batch temperature at about 75 °C to 80 °C;
7. Charge 0.109× (by mass) of deionized water to reactor 1 as a rinse;
8. Charge 0.271× (by mass) of isopropanol or 2-propanol to reactor 1 as a rinse;
9. Agitate and bring the contents of reactor 1 to 75 to 80 °C;
10. Transfer contents of reactor 1 to reactor 2 through a ≤1.0-micron filter, maintaining the batch temperature at about 75 to 80 °C;

### Procedure for the Formation, Crystallization and Isolation of the Sitagliptin-Propyl Gallate Complex or Composition (Wherein X is the Mass (Weight) of Sitagliptin Dihydrogen Phosphate Monohydrate):

11. Cool contents of reactor 2 to seed point temperature of about 65 °C to 67 °C;
12. Charge 0.005× (by mass) of sitagliptin dihydrogen phosphate monohydrate milled seed to reactor 2;
13. Agitate and age seed bed from Step 12 for about 2.75 hours to 5 hours at temperature of about 65 °C to 67 °C;
14. Ramp cool contents of reactor 2 to about 15 °C to 25 °C at a rate of −7.2 °C per hour;
15. Add Propyl Gallate as follows: Charge 1.05× (by mass) of IPA containing 0.172× (by mass) Propyl Gallate over about 1.5 hours to 3 hours to reactor 2; OR
16. Alternatively, add Propyl Gallate as a concentrated solution in IPA: make 1.451× (by mass) of 15 wt % Propyl Gallate in IPA solution in a separate vessel and transfer to reactor 2;
17. Ramp cool contents of reactor 2 to about −10 to 0 °C at a rate of −9.6 °C per hour;
18. Agitate and age the contents of reactor 2 for a minimum of 1 hour:
19. Isolate the resulting sitagliptin-antioxidant complex via filtration to form wet cake:
20. The wet cake is optionally washed with a cold wash solution comprising propyl gallate in aqueous isopropanol; and
21. Dry the sitagliptin-antioxidant complex under vacuum at a temperature of ≤45 °C. Additionally, the dry sitagliptin-antioxidant complex/cake may be de-lumped and blended.

In one embodiment, in Step 3 of the dissolution procedure the temperature is about 20 °C. In another embodiment, dry propyl gallate can be added in Step 4 of the dissolution procedure. In another embodiment, in Step 5 of the dissolution procedure the temperature is about 70 °C to 80 °C. In another embodiment, in Step 6 of the dissolution procedure the temperature is about 78 °C. In another embodiment, in Step 9 of the dissolution procedure the temperature is about 78 °C. In another embodiment, in Step 10 of the dissolution procedure the temperature is about 78 °C. In another embodiment, in Step 13 of the crystallization/isolation procedure the seed bed is aged for about 3 hours. In another embodiment, in Step 14 of the crystallization/isolation procedure the contents of reactor 2 are cooled to about 20 °C. In another embodiment, in Step 14 of the crystallization/isolation procedure the contents of reactor 2 are cooled for about 6.5 hours. In another embodiment, in Step 15 of the crystallization/isolation procedure the propyl gallate is added over about 2 hours. In another embodiment, in Step 15 of the crystallization/isolation procedure the contents of reactor 2 are cooled for about 2.5 hours. In another embodiment of Step 16 of the crystallization/isolation procedure, the 15 wt % propyl gallate in IPA solution comprises 0.218× (by mass) PG in 1.234× (by mass) IPA. In another embodiment, in Step 17 of the crystallization/isolation procedure the contents of reactor 2 are cooled to about −5 °C. In another embodiment, in Step 17 of the crystallization/isolation procedure the contents of reactor 2 are cooled for about 2.5 hours. In another embodiment, in optional Step 20 of the crystallization/isolation procedure the temperature of the cold wash solution of propyl gallate in aqueous isopropanol is about −10 °C to 0 °C. In another embodiment, in optional Step 20 of the crystallization/isolation procedure the cold wash solution comprises 3.6 wt % propyl gallate in a 10:90 water:isopropanol solution.

In another embodiment, in optional Step 20 of the crystallization/isolation procedure the cold wash solution comprises about 0.193× (by mass) deionized water, 1.735× (by mass) isopropanol, and 0.072× (by mass) propyl gallate. In another embodiment, in Step 21 of the crystallization/isolation procedure the batch is dried at a temperature of about 40 °C. In another embodiment, the propyl gallate is replaced with butylated hydroxyanisole as the antioxidant.

### Example 1

### Sitagliptin Dihydrogen Phosphate Monohydrate-Propyl Gallate Complex or Composition

TABLE 1

| | | Purity |
|---|---|---|
| MATERIALS | CAS# | (w/w %) |
| Sitagliptin Dihydrogen Phosphate | 486460-32-6 | 100 |
| Monohydrate | | |
| Sitagliptin Dihydrogen Phosphate | 486460-32-6 | 100 |
| Monohydrate Milled Seed | | |
| Isopropyl Alcohol (2-Propanol or | 67-63-0 | 100 |
| IPA) | | |
| Deionized (DI) Water | 7732-18-5 | 100 |
| Propyl Gallate (PG) | 121-79-9 | 100 |

The sitagliptin dihydrogen phosphate monohydrate-propyl gallate complex or composition was formed using the above starting materials and the following procedure.

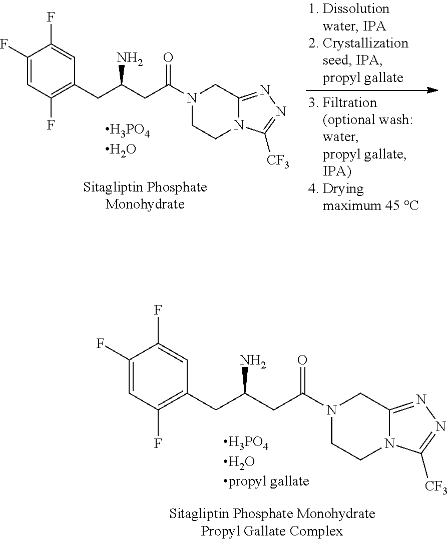 [SMILES: N[C@@H](CC(=O)N1CCn2c(nnc2C(F)(F)F)C1)Cc1cc(F)c(F)cc1F ; N[C@@H](CC(=O)N1CCn2c(nnc2C(F)(F)F)C1)Cc1cc(F)c(F)cc1F]

A vessel was charged with sitagliptin dihydrogen phosphate monohydrate (15 g) and a solution of 71:29 w/w isopropanol:water (53.2 g). The mixture was heated to 78 °C to dissolve the sitagliptin dihydrogen phosphate monohydrate. Then the mixture was cooled to 66 °C, and pin-milled sitagliptin dihydrogen phosphate monohydrate seed (77 mg) was added. The resulting seed bed mixture was aged for 3 hours, then cooled to 20 °C with a cooling rate of 7.2 °C per hour. 2-propanol (15.75 g) was added to the mixture over 2 hours. A solution of 20 wt % propyl gallate (20 g) in 2-propanol (80 g) was prepared. Then the 20 wt % propyl gallate/2-propanol solution (22.98 g) was added to the seed bed mixture to give a 5 wt % propyl gallate/2-propanol solution in the mother liquor. Then the seed bed mixture was cooled to −5 °C with a cooling rate of 9.6 °C per hour. The resulting slurry was aged for 1 hour, followed by filtration through a filter funnel. The solids in the filter funnel were isolated and dried in a vacuum oven at 40 °C for 3 days to give the sitagliptin dihydrogen phosphate monohydrate propyl gallate complex. PG Assay: 0.9736 wt %.

### Example 2

### Sitagliptin Dihydrogen Phosphate Monohydrate-Butylated Hydroxyanisole Complex or Composition

TABLE 2

| | Purity |
|---|---|
| MATERIALS | (w/w %) |
| Sitagliptin Dihydrogen Phosphate | 100 |
| Monohydrate | |
| Sitagliptin Dihydrogen Phosphate | 100 |
| Monohydrate Milled Seed | |
| Isopropyl Alcohol (2-Propanol or | 100 |
| IPA) | |
| Deionized (DI) Water | 100 |
| Butylated Hydroxyanisole (BHA) | 100 |

The sitagliptin dihydrogen phosphate monohydrate-butylated hydroxyanisole complex or composition was formed using the above starting materials and the following procedure.

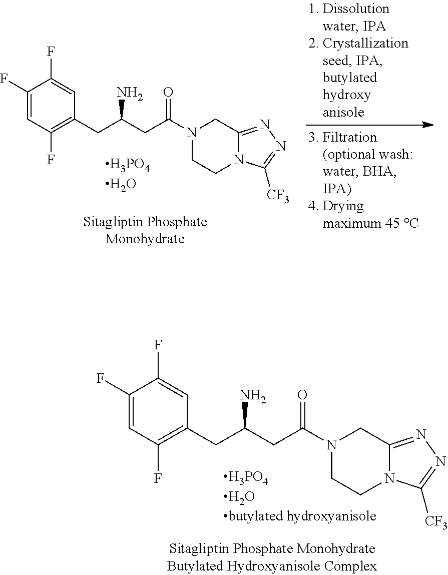 [SMILES: N[C@@H](CC(=O)N1CCn2c(nnc2C(F)(F)F)C1)Cc1cc(F)c(F)cc1F ; N[C@@H](CC(=O)N1CCn2c(nnc2C(F)(F)F)C1)Cc1cc(F)c(F)cc1F]

A vessel was charged with sitagliptin dihydrogen phosphate monohydrate (15.002 g) and a 71:29 w/w IPA:water solution (53.2 g). The mixture was heated to 80 °C to dissolve the resulting slurry. Then the resulting solution was cooled to the seeding temperature of 66 °C. The pin-milled sitagliptin dihydrogen phosphate monohydrate seed (76 mg) was added to the solution and the seed bed mixture was aged for 3 hours. The seed bed mixture was then cooled to 20 °C at a cooling rate of 7.2 °C per hour. 2-propanol (15.75 g) was added to the mixture over 2 hours. Then a solution of 10 wt % butylated hydroxyanisole in 2-propanol (17.2 g) was added to the seed bed mixture to give a 2 wt % BHA solution in the mother liquor. The resulting mixture was cooled to −5 °C at a cooling rate of 9.6 °C per hour. The resulting mixture was aged for one hour. The resulting solid was isolated by filtering through a filter funnel, and the wet filter cake was dried in a vacuum oven at 40 °C for about 1 day. BHA Assay: 0.411108 wt %, F

### Example 3

### NTTP Formation in JANUVIA® Core Tablets Made with Sitagliptin Dihydrogen Phosphate Monohydrate-Antioxidant Complex or Composition Powder

NTTP growth, after stability aging under the conditions shown in Table 3, in JANUVIA® core tablets made with sitagliptin dihydrogen phosphate monohydrate powder without an antioxidant (control) or with a sitagliptin dihydrogen phosphate monohydrate antioxidant complex powder, in which the antioxidant is either propyl gallate (PG) or butylated hydroxyanisole (BHA) at the wt % antioxidant concentration provided in Table 3.

TABLE 3

| | Antioxidant | | | | |
|---|---|---|---|---|---|
| | Concentration | | | | |
| | [wt. % of | | | | |
| | sitagliptin | JANUVIA ® Core Tablet | | JANUVIA ® Core Tablet | |
| | dihydrogen | Aging | | NTTP Growth [ppm] | |
| | phosphate | headspace | | Without | With |
| Antioxidant | monohydrate] | condition | time | Antioxidant | Antioxidant |
| Propyl Gallate | 0.15 | 40 C/75% RH | 1.5 months | 0.43 | 0.08 |
| | | 30 C/65% RH | 1.5 weeks | 0.06 | 0.01 |
| Butylated | 0.12 | 40 C/75% RH | 1.5 weeks | 0.13 | 0.00 |
| Hydroxyanisole | | 30 C/65% RH | 1.5 weeks | 0.06 | 0.00 |

Experimental Summary: Sitagliptin dihydrogen phosphate monohydrate powder was slurry washed 2× with a 90:10 w/w isopropanol:water solution containing either 0% (Table 3 control) or approximately 1% antioxidant (either propyl gallate or butylated hydroxyanisole). The sitagliptin dihydrogen phosphate monohydrate powder and sitagliptin dihydrogen phosphate monohydrate antioxidant complex powders were drained, and then dried. The sitagliptin-antioxidant complex powder was confirmed by assay. JANUVIA® (sitagliptin 100-mg dose) core tablets (13/32” diameter concave image) were compressed at 200 MPa after blending excipients with: 1) sitagliptin dihydrogen phosphate monohydrate powder (control); 2) sitagliptin dihydrogen phosphate monohydrate propyl gallate complex powder (PG; prepared in a procedure similar to the procedure of Example 1); or 3) sitagliptin dihydrogen phosphate monohydrate butylated hydroxyanisole complex powder (BHA; prepared in a procedure similar to the procedure of Example 2). The compressed tablets were aged under pseudo-open-dish conditions, with controlled humidity, temperature and time, as noted in Table 3. Samples were then stored at −20 °C until analytical testing to measure the amount of NTTP formed in each tablet.

Table 3 shows a reduction in the formation of NTTP in JANUVIA® core tablets using either a sitagliptin dihydrogen phosphate monohydrate propyl gallate complex powder or sitagliptin dihydrogen phosphate monohydrate butylated hydroxyanisole complex powder relative to JANUVIA® core tablets using sitagliptin dihydrogen phosphate monohydrate powder without an antioxidant (control).

### Example 4

### NTTP Reduction in JANUVIA® Core Tablets Containing Sitagliptin Dihydrogen Phosphate Monohydrate-Antioxidant Complex or Composition Powder

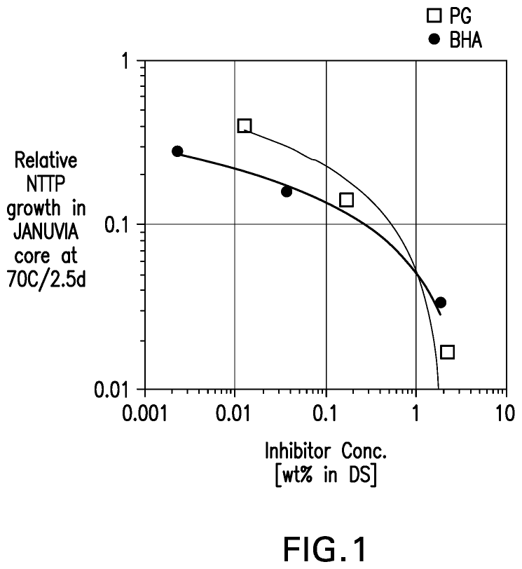

FIG. 1 shows the reduction of NTTP growth after accelerated aging at 70 °C for 2.5 days in JANUVIA®) core tablets made with sitagliptin dihydrogen phosphate monohydrate-antioxidant complex powder as a function of antioxidant concentration in the sitagliptin dihydrogen phosphate monohydrate-antioxidant complex powder. The NTTP growth is reported as a fraction of the NTTP growth of 0.6 ppm seen in tablets with unmodified sitagliptin dihydrogen phosphate monohydrate, the same ingredient/excipient lots and identical processing and stability aging. The lines are logarithmic fits to guide the eye.

FIG. 1 Experimental Summary: Sitagliptin dihydrogen phosphate monohydrate powder was slurry washed with a 90:10 w/w isopropanol:water solution containing antioxidant concentrations of either 10%, 1% or 0.1% by weight (or wt %) of propyl gallate or butylated hydroxyanisole as the drug substance (or wt % DS). The resulting powder was then drained and dried to yield sitagliptin dihydrogen phosphate monohydrate propyl gallate complex powder (PG) and sitagliptin dihydrogen phosphate monohydrate butylated hydroxyanisole complex powder (BHA) (which were confirmed by assay). JANUVIA® (sitagliptin free amine 100-mg dose) core tablets (13/32” diameter concave image) were compressed at 200 MPa after blending excipients with: 1) sitagliptin dihydrogen phosphate monohydrate powder (control DS), and 2) sitagliptin dihydrogen phosphate monohydrate antioxidant complex powder wherein the antioxidant was either propyl gallate (PG DS) or butylated hydroxyanisole (BHA DS). The compressed tablets were packaged in heat-sealed aluminum foil sachets and aged for approximately 60 hours at 70 °C. The tablet samples were stored at −20 °C until analytical testing to measure NTTP growth, which was 0.6 ppm for tablets with unmodified sitagliptin dihydrogen phosphate monohydrate powder (control DS without propyl gallate and butylated hydroxyanisole).

FIG. 1. shows a reduction in the formation of NTTP in JANUVIA®) core tablets using either a sitagliptin dihydrogen phosphate monohydrate propyl gallate complex powder (PG) or a sitagliptin dihydrogen phosphate monohydrate butylated hydroxyanisole complex powder (BHA) relative to JANUVIA® core tablets using sitagliptin dihydrogen phosphate monohydrate powder without an antioxidant (control).

### Example 5

### NTTP Formation in JANUVIA®7 Core Tablets Containing a Sitagliptin Antioxidant Complex or Composition

TABLE 5

| | Antioxidant % | | |
|---|---|---|---|
| | (weight % based on sitagliptin | | NTTP |
| Product | dihydrogen phosphate monohydrate) | Condition | [ppm] |
| JANUVIA ®_100 | Antioxidant-free (no antioxidant added) | 70 °C/2.5 d (sachet) | 0.71 |
| mg | 0.016% propyl gallate | 70 °C/2.5 d (sachet) | 0.34 |
| | 0.18% propyl gallate | 70 °C/2.5 d (sachet) | 0.18 |
| | 2.0% propyl gallate | 70 °C/2.5 d (sachet) | 0.11 |
| | 0.0044% butylated hydroxyanisole | 70 °C/2.5 d (sachet) | 0.28 |
| | 0.056% butylated hydroxyanisole | 70 °C/2.5 d (sachet) | 0.19 |
| | 1.7% butylated hydroxyanisole | 70 °C/2.5 d (sachet) | 0.11 |

Experimental Summary: JANUVIA® 100 mg core tablet formulations containing sitagliptin antioxidant complex or composition were compressed into tablets and stored at 70 °C for 2.5 days.

The control (antioxidant free) formulation listed in Table 6 was prepared by dry blending sitagliptin dihydrogen phosphate monohydrate with microcrystalline cellulose, dicalcium phosphate, croscarmellose sodium, magnesium stearate, and sodium stearyl fumarate, and directly compressed to give tablets. The antioxidant free tablets were stored at 70 °C for 2.5 days to simulate 2 years at 30 °C.

The other example formulations listed in Table 5 were prepared by dry blending either 1) the sitagliptin dihydrogen phosphate monohydrate-propyl gallate complex or composition, or 2) the sitagliptin dihydrogen phosphate monohydrate-butylated hydroxyanisole complex or composition with microcrystalline cellulose, dicalcium phosphate, croscarmellose sodium, magnesium stearate, and sodium stearyl fumarate. The JANUVIA® core tablet formulations were directly compressed to give tablets, which were stored at 70 °C for 2.5 days to simulate 2 years at 30 °C.

The data in Table 5 show a decrease in NTTP formation in the JANUVIA®(core tablets with increasing propyl gallate and butylated hydroxyanisole levels. The data in Table 5 also show that the NTTP concentration decreases 1) as the propyl gallate concentration increases, and 2) as the butylated hydroxyanisole concentration increases.

### Example 6

### NTTP Formation in STEGLUJAN® Core Tablets Containing a Sitagliptin Antioxidant Complex or Composition

TABLE 6

| | Antioxidant % | | |
|---|---|---|---|
| | (weight % based on | | |
| | sitagliptin dihydrogen | | NTTP |
| Product Core | phosphate monohydrate) | Condition | [ppm] |
| STEGLUJAN ® | Antioxidant-free (no | 70 °C/2.5 days | 0.77 |
| 5 mg/100 mg | antioxidant added) | (sachet) | |
| | 2.0% Propyl Gallate | 70 °C/2.5 days | 0.11 |
| | | (sachet) | |
| | 1.7% Butylated | 70 °C/2.5 days | 0.11 |
| | | (sachet) | |
| | Hydroxyanisole | | |

Experimental Summary: STEGLUJAN® 5 mg/100 mg core tablets containing sitagliptin-antioxidant complex or composition were compressed and stored at 70 °C for 2.5 days.

The control (antioxidant free) formulation listed in Table 6 was prepared by dry blending sitagliptin dihydrogen phosphate monohydrate with ertugliflozin L-pyroglutamic acid, microcrystalline cellulose, dicalcium phosphate, croscarmellose sodium, magnesium stearate, and sodium stearyl fumarate, and directly compressed to give tablets. The antioxidant free tablets were stored at 70 °C for 2.5 days to simulate 2 years at 30 °C.

The other example formulations in Table 6 were prepared by dry blending either sitagliptin dihydrogen phosphate monohydrate propyl gallate complex or composition, or 2) sitagliptin dihydrogen phosphate monohydrate butylated hydroxyanisole complex or composition, with ertugliflozin L-pyroglutamic acid, microcrystalline cellulose, dicalcium phosphate, croscarmellose sodium, magnesium stearate, and sodium stearyl fumarate. The resulting formulations were directly compressed into tablets, which were stored at 70 °C for 2.5 days to simulate 2 years at 30 °C.

The data in Table 6 show that addition of either the sitagliptin propyl gallate complex or composition, or the sitagliptin butylated hydroxyanisole complex or composition to the STEGLUJAN® 5 mg/100 mg core tablets resulted in lower levels of NTTP than in the antioxidant free STEGLUJAN® 5 mg/100 mg core tablets after storage at 70 °C for 2.5 days.

### Example 7

### NTTP Formation in JANUVIAC, Film Coated Tablets Prepared with a Sitagliptin Propyl Gallate Complex or Composition

TABLE 7

| | Antioxidant % | | |
|---|---|---|---|
| | (weight % based on | | |
| Film Coated | sitagliptin dihydrogen | | NTTP |
| Tablets | phosphate monohydrate) | Condition | [ppm] |
| JANUVIA ® | Antioxidant-free (no | 40 °C/75% RH/ | 0.28 |
| 100 mg | antioxidant added) | 1.5 months (pseudo- | |
| | | open-dish) | |
| JANUVIA ® | Antioxidant-free (no | 40 °C/75% RH/ | 0.31 |
| 25 mg | antioxidant added) | 1.5 months (pseudo- | |
| | | open-dish) | |
| JANUVIA ® | 0.29% Propyl Gallate | 40 °C/75% RH/ | 0.05 |
| 100 mg | | 1.5 months (pseudo- | |
| | | open-dish) | |
| JANUVIA ® | 0.29% Propyl Gallate | 40 °C/75% RH/ | 0.11 |
| 25 mg | | 1.5 months (pseudo- | |
| | | open-dish) | |

Experimental Summary: JANUVIA® 25 mg and 100 mg film coated tablets containing sitagliptin-propyl gallate complex or composition were stored at 40 °C and at 75% relative humidity for 1.5 months.

The control (antioxidant free) formulations listed in Table 7 were prepared dry blending sitagliptin dihydrogen phosphate monohydrate with microcrystalline cellulose, dicalcium phosphate, croscarmellose sodium, magnesium stearate, and sodium stearyl fumarate, and directly compressed to give tablets. The antioxidant free tablets were film coated, and stored at 40 °C and 75% RH for 1.5 months.

The other examples in Table 7 were prepared by dry blending sitagliptin dihydrogen phosphate monohydrate propyl gallate complex or composition with microcrystalline cellulose, dicalcium phosphate, croscarmellose sodium, magnesium stearate, and sodium stearyl fumarate. The resulting formulations were directly compressed into tablets, which were film coated and stored at 40 °C, and 75% relative humidity for 1.5 months.

The data in Table 7 show that propyl gallate effectively reduces NTTP formation in JANUVIAk 25 mg and 100 mg film coated tablets after aging at 40 °C and 75% relative humidity (RH) for 1.5 months.

### Example 8

### NTTP Formation in JANUVIA® Core Tablets Prepared by Dry Addition of Antioxidant

TABLE 8

| | Antioxidant % | | |
|---|---|---|---|
| | (weight % relative to | | |
| | sitagliptin dihydrogen | | NTTP |
| Product Core | phosphate monohydrate) | Condition | [ppm] |
| JANUVIA ® | Antioxidant-free (no | 70 °C/60% RH/ | 0.63 |
| 100 mg | antioxidant added) | 2.5 days (pseudo- | |
| | | open dish) | |
| | 0.125% propyl gallate | 70 °C/60% RH/ | 0.25 |
| | | 2.5 days (pseudo- | |
| | | open dish) | |
| | 0.125% butylated | 70 °C/60% RH/ | 0.22 |
| | hydroxyanisole | 2.5 days (pseudo- | |
| | | open dish) | |

Experimental Summary: Dry addition of antioxidants to JANUVIA® 100 mg core tablets stored at 70 °C and 60% relative humidity for 2.5 days.

The control (antioxidant free) formulation listed in Table 8 was prepared dry blending sitagliptin dihydrogen phosphate monohydrate with microcrystalline cellulose, dicalcium phosphate, croscarmellose sodium, magnesium stearate, and sodium stearyl fumarate, and directly compressed to give tablets. The control (antioxidant free) tablets were stored at 70 °C and 60% relative humidity for 2.5 days.

The other examples listed in Table 8 were prepared by the dry addition of either propyl gallate or butylated hydroxyanisole, each with particle sizes less than or equal to 150 μm, to sitagliptin dihydrogen phosphate monohydrate, microcrystalline cellulose, dicalcium phosphate, croscarmellose sodium, magnesium stearate, and sodium stearyl fumarate. The resulting formulations were directly compressed to form JANUVIA®-100 mg core tablets, which were stored at 70 °C and 60% relative humidity for 2.5 days.

The data in Table 8 show that the dry addition of either propyl gallate or butylated hydroxyanisole to JANUVIA® 100 mg core tablets reduces the formation of NTTP relative to the control tablets after accelerated aging at 70 °C and 60% relative humidity for 2.5 days.

### Example 9

### NTTP Formation in JANUVIA® Core Tablets Prepared by Dry Addition of Milled or Unmilled Propyl Gallate

TABLE 9

| | Propyl Gallate | | | |
|---|---|---|---|---|
| | particle size as | Propyl Gallate % | | |
| | Mean Particle | (weight % relative to | | |
| | Size (Mv) | sitagliptin phosphate | | NTTP |
| Product Core | [microns] | monohydrate) | Conditions | [ppm] |
| JANUVIA ® | Propyl Gallate- | No Antioxidant added | 60 °C/60% RH/1.5 | 1.11 |
| 100 mg | free (no propyl | | weeks? | |
| | gallate added) | | | |
| | 22 | 0.389% | 60 °C/60% RH/1.5 | 0.27 |
| | | | weeks | |
| | 22 | 0.0973% | 60 °C/60% RH/1.5 | 0.32 |
| | | | weeks | |
| | 44 | 0.389% | 60 °C/60% RH/1.5 | 0.25 |
| | | | weeks | |
| | 44 | 0.0973% | 60 °C/60% RH/1.5 | 0.39 |
| | | | weeks | |
| | 83 | 0.389% | 60 °C/60% RH/1.5 | 0.28 |
| | | | weeks | |
| | 83 | 0.0973% | 60 °C/60% RH/1.5 | 0.46 |
| | | | weeks | |
| | 375 unmilled | 1.56% | 60 °C/60% RH/1.5 | 0.28 |
| | | | weeks | |
| | 375 unmilled | 0.778% | 60 °C/60% RH/1.5 | 0.39 |
| | | | weeks | |
| | 375 unmilled | 0.389% | 60 °C/60% RH/1.5 | 0.51 |
| | | | weeks | |

Experimental Summary: Unmilled or milled propyl gallate at various concentrations was dry blended into the JANUVIA®, 100 mg core tablets, which were stored at 60 °C and 60% relative humidity for 1.5 weeks.

The control (propyl gallate free) formulation listed in Table 9 was prepared dry blending sitagliptin dihydrogen phosphate monohydrate, microcrystalline cellulose, dicalcium phosphate, croscarmellose sodium, magnesium stearate, and sodium stearyl fumarate, and directly compressed to give tablets. The control (propyl gallate free) tablets were stored at 60 °C and 60% relative humidity for 1.5 weeks.

The other example formulations in Table 9 were prepared by dry blending unmilled propyl gallate (Mv=375 μm) or propyl gallate pin-milled to smaller particle sizes of Mv=83 μm, 44 μm, or 22 μm as measured by static light scattering). The unmilled propyl gallate or milled propyl gallate was dry blended with sitagliptin dihydrogen phosphate monohydrate, microcrystalline cellulose, dicalcium phosphate, croscarmellose sodium, magnesium stearate, and sodium stearyl fumarate. The resulting formulations were directly compressed to give JANUVIA® 100 mg core tablets, which were stored at 60 °C and 60% relative humidity for 1.5 weeks.

The data in Table 9 show that both unmilled and milled propyl gallate dry blended into the JANUVIA® 100 mg core tablet formulation effectively suppresses NTTP formation in the core tablet relative to the control tablet.

Table 9 also shows that, when the propyl gallate particle size is reduced, a lower propyl gallate concentration is required to achieve the same reduction in NTTP formation in the JANUVIA® 100 mg core tablet.

### Example 10

### NTTP Formation in STEGLUJAN® Core Tablets Prepared by Dry Addition of Antioxidant

TABLE 10

| | Propyl Gallate % | | |
|---|---|---|---|
| | (weight % relative to | | |
| | sitagliptin phosphate | | NTTP |
| Product Core | monohydrate) | Conditions | [ppm] |
| STEGLUJAN ® | Propyl Gallate free (no | 60 °C/30% RH/ | 0.63 |
| 5 mg/100 mg | propyl gallate added) | 1.5 weeks (pseudo- | |
| | | open dish) | |
| | 1.56% propyl gallate | 60 °C/30% RH/ | 0.21 |
| | | 1.5 weeks (pseudo- | |
| | | open dish) | |

Experimental Summary: Propyl gallate was added by dry addition to the STEGLUJAN® 5 mg/100 mg core tablet formulation, which was stored at 60 °C and 30% relative humidity for 1.5 weeks.

The control (propyl gallate free) formulation listed in Table 10 was prepared dry blending sitagliptin dihydrogen phosphate monohydrate, ertugliflozin L-pyroglutamic acid, microcrystalline cellulose, dicalcium phosphate, croscarmellose sodium, magnesium stearate and sodium stearyl fumarate, and directly compressed to give tablets. The control (propyl gallate free) tablets were stored at 60 °C and 30% RH for 1.5 weeks.

The other example formulation listed in Table 10 was prepared as follows. Propyl gallate particles were dispersed via mortar and pestle and pushing antioxidant particles through a 150 μm sieve. The dispersed propyl gallate particles were dry blended with sitagliptin dihydrogen phosphate monohydrate, ertugliflozin L-pyroglutamic acid, microcrystalline cellulose, dicalcium phosphate, croscarmellose sodium, magnesium stearate and sodium stearyl fumarate. The core tablets were prepared with this formulation, and stored at 60 °C and 30% RH for 1.5 weeks, which is intended to simulate 2 years at 30 °C.

The data in Table 10 show that the dry addition of propyl gallate to the STEGLUJAN® 5 mg/100 mg core tablet formulation, stored at 60 °C and 30% RH for 1.5 weeks, effectively reduced the formation of NTTP in the core tablet formulation relative to the control tablet.

### Example 11

### NTTP Formation in JANUVIA® Core Tablets Prepared by Liquid Addition of Antioxidant

TABLE 11

| | Propyl Gallate % | | |
|---|---|---|---|
| | (weight % relative to | | |
| | sitagliptin phosphate | | NTTP |
| Tablet Core | monohydrate) | Condition | [ppm] |
| JANUVIA ® | Propyl Gallate Free | 60 °C/60% RH/ | 0.97 |
| 100 mg | (No propyl gallate | 1.5 weeks (pseudo- | |
| | added) | open dish) | |
| | 0.28% propyl gallate | 60 °C/60% RH/ | 0.21 |
| | | 1.5 weeks (pseudo- | |
| | | open dish) | |

Experimental Summary: Propyl gallate was added by liquid addition onto dibasic calcium phosphate, which was added to sitagliptin 100 mg formulation tablet cores and stored at 60 °C and 60% relative humidity for 1.5 weeks.

The control (propyl gallate free) formulation listed in Table 9 was prepared dry blending sitagliptin dihydrogen phosphate monohydrate with microcrystalline cellulose, dicalcium phosphate, croscarmellose sodium, magnesium stearate, and sodium stearvl fumarate, and directly compressed to give tablets. The control (propyl gallate free) tablets were stored at 60 °C and 60% relative humidity for 1.5 weeks.

The other formulation example in Table 11 was prepared by the addition of propyl gallate to the core tablet formulation as a liquid. Propyl gallate was dissolved with PEG400 (PG:PEG400 1:1 (w/w)) in non-denatured ethanol. The ethanol solution was added to dicalcium phosphate, fully wetting the dicalcium phosphate with minimal excess ethanol. The ethanol was then evaporated leaving propyl gallate/PEG400 on dicalcium phosphate, which was dry blended with sitagliptin dihydrogen phosphate monohydrate, microcrystalline cellulose, croscarmellose sodium, magnesium stearate, and sodium stearyl fumarate. The JANUVIA® core tablets were prepared, stored at 60 °C for 1.5 weeks and NTTP formation was determined.

The data in Table 11 show that the addition of liquid propyl gallate on dicalcium phosphate to the JANUVIA®100 mg core table formulation effectively suppresses NTTP formation in the core tablet.

While preferred embodiments of the present disclosure have been shown and described herein, it will be obvious to those skilled in the art that such embodiments are provided by way of example only. Numerous variations, changes, and substitutions will now occur to those skilled in the art without departing from the present disclosure. It should be understood that various alternatives to the embodiments of the present disclosure described herein may be employed in practicing the methods and uses disclosed herein.

1. A composition comprising:
(a) about 90% to 99.99% by weight of a sitagliptin, or a pharmaceutically acceptable salt and/or hydrate thereof, and
(b) about 0.01% to 10% by weight of an antioxidant.

2. The composition of claim 1 comprising:
(a) about 95% to 99.95% by weight of a sitagliptin, or a pharmaceutically acceptable salt and/or hydrate thereof, and
(b) about 0.05% to 5% by weight of an antioxidant.

3. The composition of claim 1 comprising:
(a) about 99% to 99.9% by weight of a sitagliptin, or a pharmaceutically acceptable salt and/or hydrate thereof, and
(b) about 0.1% to 1% by weight of an antioxidant.

4. The composition of claim 1 comprising:
(a) about 99.3% to 99.8% by weight of a sitagliptin, or a pharmaceutically acceptable salt and/or hydrate thereof, and
(b) about 0.2% to 0.7% by weight of an antioxidant.

5. The composition of claim 1 wherein the salt of sitagliptin is sitagliptin dihydrogen phosphate.

6. The composition of claim 1 wherein the salt and hydrate of sitagliptin is sitagliptin dihydrogen phosphate monohydrate.

7. The composition of claim 1 wherein the antioxidant is selected from: ascorbic acid, methyl gallate, ethyl gallate, propyl gallate, butyl gallate, pentyl gallate, octyl gallate, dodecyl gallate, lauryl gallate, butylated hydroxyanisole, butylated hydroxytoluene, thymol, tocopherol, and vitamin E.

8. The composition of claim 1 wherein the antioxidant is selected from: ascorbic acid, propyl gallate, butylated hydroxyanisole, butylated hydroxytoluene, thymol, tocopherol, and vitamin E.

9. The composition of claim 1 wherein the antioxidant is propyl gallate.

10. The composition of claim 1 wherein the antioxidant is butylated hydroxyanisole.

11. A process of preparing a sitagliptin and antioxidant composition of claim 1 of preparing a sitagliptin and antioxidant complex or composition comprising the steps of:
1) dissolving sitagliptin dihydrogen phosphate monohydrate in a suitable solvent at suitable temperature to provide a solution;
2) adding sitagliptin dihydrogen phosphate monohydrate seed to the solution of Step 1;
3) adding anti-solvent to the solution of Step 2 to form a mixture;
4) adding an antioxidant to the mixture of Step 3 to form a mixture;
5) cooling the mixture of Step 4; and
6) filtering the mixture of Step 5 to isolate the solid sitagliptin dihydrogen phosphate monohydrate-antioxidant complex or composition.

12. The process of claim 11 comprising the steps of:
1) dissolving sitagliptin dihydrogen phosphate monohydrate in a suitable solvent at suitable temperature to provide a solution;
2) cooling the solution of Step 1 to the seed point temperature;
3) adding sitagliptin dihydrogen phosphate monohydrate milled seed to the solution of Step 2;
4) cooling the solution of Step 3 to about 15 °C to 25 °C;
5) adding anti-solvent to the solution of Step 4 to form a mixture;
6) adding an antioxidant to the mixture of Step 5 to form a mixture;
7) cooling the mixture of Step 6 to about −10 to 0 °C;
8) filtering the mixture of Step 7 to isolate the sitagliptin dihydrogen phosphate monohydrate-antioxidant complex; and
9) drying the sitagliptin dihydrogen phosphate monohydrate-antioxidant complex.

13. The process of claim 12, further comprising de-lumping and blending the sitagliptin dihydrogen phosphate monohydrate-antioxidant complex of Step 9.

14. The process of claim 12 further comprising washing the wet sitagliptin dihydrogen phosphate monohydrate antioxidant complex with a cold wash solution comprising propyl gallate in aqueous isopropanol after filtering in Step 8.

15. The process of claim 12 wherein the suitable solvent in Step 1 is a mixture of water and isopropanol, and the suitable temperature in Step 1 is about 65 °C to 80 °C.

16. The process of claim 12 wherein the suitable solvent in Step 1 is a mixture of a 79:21 of isopropanol:water, and the suitable temperature in Step 1 is about 70 °C to 80 °C.

17. The process of claim 12 wherein the seed point temperature in Step 2 is about 65 °C to 67 °C.

18. The process of claim 12 wherein the antisolvent of Step 4 is isopropanol.

19. The process of claim 12 wherein the antioxidant of Step 6 is propyl gallate; and the sitagliptin dihydrogen phosphate monohydrate antioxidant complex of Step 9 is the sitagliptin dihydrogen phosphate monohydrate propyl gallate complex.

20. The process of claim 12 wherein the antioxidant of Step 6 is butylated hydroxyanisole; and the sitagliptin dihydrogen phosphate monohydrate antioxidant complex of Step 9 is the sitagliptin dihydrogen phosphate monohydrate butylated hydroxyanisole complex.

21. The process of claim 12 wherein the sitagliptin dihydrogen phosphate monohydrate-antioxidant complex of Steps 8 and 9 is a solid.

22. The process of claim 12 wherein the sitagliptin dihydrogen phosphate monohydrate-antioxidant complex of Steps 8 and 9 is a crystalline solid.

23. A pharmaceutical composition comprising
a) the composition of claim 1; and
b) at least one excipient selected from: microcrystalline cellulose, anhydrous dibasic calcium phosphate, croscarmellose sodium, magnesium stearate, and sodium stearyl fumarate.

24. A pharmaceutical composition comprising
a) the composition of claim 1;
b) microcrystalline cellulose;
c) anhydrous dibasic calcium phosphate;
d) croscarmellose sodium;
e) magnesium stearate; and
f) sodium stearyl fumarate.

25. A pharmaceutical composition comprising
a) the composition of claim 1;
b) microcrystalline cellulose;
c) anhydrous dibasic calcium phosphate;
d) croscarmellose sodium;
e) magnesium stearate; and
f) sodium stearyl fumarate;
wherein the composition of claim 1 is a sitagliptin dihydrogen phosphate monohydrate-butylated hydroxyanisole composition.

26. A pharmaceutical composition comprising
a) the composition of claim 1;
b) microcrystalline cellulose;
c) anhydrous dibasic calcium phosphate;
d) croscarmellose sodium;
e) magnesium stearate; and
f) sodium stearyl fumarate;
wherein the composition of claim 1 is a sitagliptin dihydrogen phosphate monohydrate-propyl gallate composition.

27. A pharmaceutical composition comprising
a) 10-50 weight % sitagliptin dihydrogen phosphate monohydrate;
b) 0.001-1 weight % antioxidant;
c) 10-50 weight % microcrystalline cellulose;
d) 10-50 weight % anhydrous dibasic calcium phosphate;
e) 0.5-5 weight % croscarmellose sodium;
f) 0.5-5 weight % sodium stearyl fumarate; and
g) 0-2 weight % magnesium stearate.

28. The pharmaceutical composition of claim 27 comprising 32.13, 64.25, or 128.5 mg of sitagliptin phosphate monohydrate.

29. The pharmaceutical composition of claim 27 further comprising a film coating.

30. A pharmaceutical composition comprising the sitagliptin and antioxidant composition of claim 1.

31. The pharmaceutical composition of claim 27 comprising an antioxidant deposited on an excipient.

32. The pharmaceutical composition of claim 31 wherein the antioxidant is propyl gallate and the excipient is dicalcium phosphate.

33. The pharmaceutical composition of claim 27 comprising an antioxidant as a dry powder.

34. The pharmaceutical composition of claim 33 wherein the antioxidant has a particle size between 1 to 400 microns.

35. A pharmaceutical composition comprising
a) the composition of claim 1;
b) ertugliflozin, or a pharmaceutically acceptable salt or co-crystal thereof; and
c) at least one excipient selected from: microcrystalline cellulose, dibasic calcium phosphate anhydrous, croscarmellose sodium, sodium stearyl fumarate, and magnesium stearate.

36. A pharmaceutical composition comprising
a) the composition of claim 1;
b) ertugliflozin, or a pharmaceutically acceptable salt or co-crystal thereof;
c) microcrystalline cellulose;
d) dibasic calcium phosphate anhydrous;
e) croscarmellose sodium;
f) sodium stearyl fumarate; and
g) magnesium stearate.

37. The pharmaceutical composition of claim 1 wherein the composition is a sitagliptin dihydrogen phosphate monohydrate-butylated hydroxyanisole composition.

38. The pharmaceutical composition of claim 1 wherein the composition is a sitagliptin dihydrogen phosphate monohydrate-propyl gallate composition.

39. The pharmaceutical composition of claim 35 comprising
a) 10-50 weight % of sitagliptin dihydrogen phosphate monohydrate;
b) 0.5-15 weight % ertugliflozin L-pyroglutamic acid co-crystal;
c) 0.001-1 weight % antioxidant;
d) 10-50 weight % microcrystalline cellulose;
e) 10-50 weight % dibasic calcium phosphate anhydrous;
f) 0.5-5 weight % croscarmellose sodium;
g) 0.5-5 weight % sodium stearyl fumarate; and
h) 0.5-2 weight % magnesium stearate.

40. The pharmaceutical composition of claim 39 comprising 128.5 mg of sitagliptin phosphate monohydrate.

41. The pharmaceutical composition of claim 39 comprising 6.48 mg, or 19.43 mg of ertugliflozin L-pyroglutamic acid co-crystal.

42. The pharmaceutical composition of claim 39 further comprising a film coating.

43. A pharmaceutical composition of claim 39 comprising a sitagliptin and antioxidant composition comprising:
(a) about 90% to 99.99% by weight of a sitagliptin, or a pharmaceutically acceptable salt and/or hydrate thereof, and
(b) about 0.01% to 10% by weight of an antioxidant.

44. The pharmaceutical composition of claim 39 comprising an antioxidant deposited on an excipient.

45. The pharmaceutical composition of claim 44 wherein the antioxidant is propyl gallate and the excipient is dicalcium phosphate.

46. The pharmaceutical composition of claim 39 comprising an antioxidant as a dry powder.

47. The pharmaceutical composition of claim 46 wherein the antioxidant has a particle size between 1 to 400 microns.

48. A core tablet comprising sitagliptin prepared using the sitagliptin and antioxidant composition of claim 1, wherein the NTTP level is less than or equal to 4 ppm after storing at 70 °C for 2.5 days.

49. The core tablet of claim 48 wherein the NTTP level is less than or equal to 1 ppm after storing at 70 °C for 2.5 days.

50. The core tablet of claim 48 wherein the NTTP level is less than or equal to 0.37 ppm after storing at 70 °C for 2.5 days.

51. The core tablet of claim 48 wherein the antioxidant is propyl gallate or butylated hydroxyanisole.

52. A core tablet comprising sitagliptin prepared using the sitagliptin and antioxidant composition of claim 1, wherein the NTTP level is less than or equal to 4 ppm after storing at 70 °C for 2.5 days, and wherein sitagliptin is equivalent to 25 mg sitagliptin free base, 50 mg sitagliptin free base, or 100 mg sitagliptin free base.

53. The core tablet of claim 52 further comprising a film coating.

54. A core tablet comprising sitagliptin prepared using an antioxidant deposited on an excipient, wherein the NTTP level is less than or equal to 4 ppm after storing at 70 °C for 2.5 days.

55. The core tablet of claim 54 wherein the NTTP level is less than or equal to 1 ppm after storing at 70 °C for 2.5 days.

56. The core tablet of claim 54 wherein the NTTP level is less than or equal to 0.37 ppm after storing at 70 °C for 2.5 days.

57. The core tablet of claim 54 wherein the antioxidant is propyl gallate and the excipient is dicalcium phosphate.

58. A core tablet comprising sitagliptin prepared using an antioxidant deposited on an excipient, wherein the NTTP level is less than or equal to 4 ppm after storing at 70 °C for 2.5 days, and wherein sitagliptin is equivalent to 25 mg sitagliptin free base, 50 mg sitagliptin free base, or 100 mg sitagliptin free base.

59. The core tablet of claim 58 further comprising a film coating.

60. A core tablet comprising sitagliptin prepared using an antioxidant as a dry powder, wherein the NTTP level is less than or equal to 4 ppm after storing at 70 °C for 2.5 days.

61. The core tablet of claim 60 wherein the NTTP level is less than or equal to 1 ppm after storing at 70 °C for 2.5 days.

62. The core tablet of claim 60 wherein the NTTP level is less than or equal to 0.37 ppm after storing at 70 °C for 2.5 days.

63. The core tablet of claim 60 wherein the antioxidant is propyl gallate or butylated hydroxyanisole.

64. A core tablet comprising sitagliptin prepared using an antioxidant as a dry powder, wherein the NTTP level is less than or equal to 4 ppm after storing at 70 °C for 2.5 days, and wherein sitagliptin is equivalent to 25 mg sitagliptin free base, 50 mg sitagliptin free base, or 100 mg sitagliptin free base.

65. The core tablet of claim 64 further comprising a film coating.

66. A core tablet comprising sitagliptin and ertugliflozin prepared using the sitagliptin and antioxidant composition of claim 1, wherein the NTTP level is less than or equal to 4 ppm after storing at 70 °C for 2.5 days.

67. The core tablet of claim 66 wherein the NTTP level is less than or equal to 1 ppm after storing at 70 °C for 2.5 days.

68. The core tablet of claim 66 wherein the NTTP level is less than or equal to 0.37 ppm after storing at 70 °C for 2.5 days.

69. The core tablet of claim 66 wherein the antioxidant is propyl gallate or butylated hydroxyanisole.

70. A core tablet comprising sitagliptin and ertugliflozin prepared using the sitagliptin and antioxidant composition of claim 1, wherein the NTTP level is less than or equal to 4 ppm after storing at 70 °C for 2.5 days, and wherein ertugliflozin is equivalent to 5 mg ertugliflozin and sitagliptin is equivalent to 100 mg sitagliptin free base; or ertugliflozin is equivalent to 15 mg ertugliflozin and sitagliptin is equivalent to 100 mg sitagliptin free base.

71. The core tablet of claim 70 further comprising a film coating.

72. A core tablet comprising sitagliptin and ertugliflozin prepared using an antioxidant deposited on an excipient, wherein the NTTP level is less than or equal to 4 ppm after storing at 70 °C for 2.5 days.

73. The core tablet of claim 72 wherein the NTTP level is less than or equal to 1 ppm after storing at 70 °C for 2.5 days.

74. The core tablet of claim 72 wherein the NTTP level is less than or equal to 0.37 ppm after storing at 70 °C for 2.5 days.

75. The core tablet of claim 72 wherein the antioxidant is propyl gallate and the excipient is dicalcium phosphate.

76. A core tablet comprising sitagliptin and ertugliflozin prepared using an antioxidant deposited on an excipient, wherein the NTTP level is less than or equal to 4 ppm after storing at 70 °C for 2.5 days, and wherein ertugliflozin is equivalent to 5 mg ertugliflozin and sitagliptin is equivalent to 100 mg sitagliptin free base; or ertugliflozin is equivalent to 15 mg ertugliflozin and sitagliptin is equivalent to 100 mg sitagliptin free base.

77. The core tablet of claim 76 further comprising a film coating.

78. A core tablet comprising ertugliflozin and sitagliptin prepared using an antioxidant as a dry powder, wherein the NTTP level is less than or equal to 4 ppm after storing at 70 °C for 2.5 days.

79. The core tablet of claim 78 wherein the NTTP level is less than or equal to 1 ppm after storing at 70 °C for 2.5 days.

80. The core tablet of claim 78 wherein the NTTP level is less than or equal to 0.37 ppm after storing at 70 °C for 2.5 days.

81. The core tablet of claim 78 wherein the antioxidant is propyl gallate or butylated hydroxyanisole.

82. A core tablet comprising sitagliptin and ertugliflozin prepared using an antioxidant as a dry powder, wherein the NTTP level is less than or equal to 4 ppm after storing at 70 °C for 2.5 days, and wherein ertugliflozin is equivalent to 5 mg ertugliflozin and sitagliptin is equivalent to 100 mg sitagliptin free base; or ertugliflozin is equivalent to 15 mg ertugliflozin and sitagliptin is equivalent to 100 mg sitagliptin free base.

83. The core tablet of claim 82 further comprising a film coating.

84. A pharmaceutical composition comprising sitagliptin, microcrystalline cellulose and an antioxidant wherein the level of NTTP is less than or equal to 4 ppm after storing at 70 °C for 2.5 days.

85. The pharmaceutical composition of claim 84 wherein the NTTP level is less than or equal to 1 ppm after storing at 70 °C for 2.5 days.

86. The pharmaceutical composition of claim 84 wherein the NTTP level is less than or equal to 0.37 ppm after storing at 70 °C for 2.5 days.

87. The pharmaceutical composition of claim 84 wherein the antioxidant is propyl gallate or butylated hydroxyanisole.

88. A pharmaceutical composition comprising sitagliptin, microcrystalline cellulose and an antioxidant wherein the level of NTTP is less than or equal to 4 ppm after storing at 70 °C for 2.5 days prepared
1) using the sitagliptin and antioxidant composition of claim 1;
2) using an antioxidant as a dry powder; or
3) using an antioxidant deposited on an excipient.

89. The pharmaceutical composition of claim 84 wherein the excipient is dicalcium phosphate.

90. A pharmaceutical composition comprising sitagliptin, dicalcium phosphate and an antioxidant wherein the level of NTTP is less than or equal to 4 ppm after storing at 70 °C for 2.5 days.

91. The pharmaceutical composition of claim 90 wherein the NTTP level is less than or equal to 1 ppm after storing at 70 °C for 2.5 days.

92. The pharmaceutical composition of claim 90 wherein the NTTP level is less than or equal to 0.37 ppm after storing at 70 °C for 2.5 days.

93. The pharmaceutical composition of claim 90 wherein the antioxidant is propyl gallate or butylated hydroxyanisole.

94. The pharmaceutical composition comprising sitagliptin, dicalcium phosphate and an antioxidant wherein the level of NTTP is less than or equal to 4 ppm after storing at 70 °C for 2.5 days prepared
1) using the sitagliptin and antioxidant composition of claim 1;
2) using an antioxidant as a dry powder; or
3) using an antioxidant deposited on an excipient.

95. The pharmaceutical composition of claim 90 wherein the excipient is dicalcium phosphate.

96. A pharmaceutical composition which is a core tablet comprising sitagliptin, or a pharmaceutically acceptable salt thereof, and an antioxidant wherein the antioxidant is added prior to tableting and wherein the level of NTTP is below 4 ppm after storing the composition at 70 °C for 2.5 days.

97. The pharmaceutical composition of claim 96 wherein the NTTP level is less than or equal to 1 ppm after storing at 70 °C for 2.5 days.

98. The pharmaceutical composition of claim 96 wherein the NTTP level is less than or equal to 0.37 ppm after storing at 70 °C for 2.5 days.

99. The pharmaceutical composition of claim 96 wherein the antioxidant is propyl gallate or butylated hydroxyanisole.

100. The pharmaceutical composition of claim 96 wherein the pharmaceutically acceptable salt of sitagliptin is sitagliptin dihydrogen phosphate.

101. The pharmaceutical composition of claim 96 wherein the pharmaceutically acceptable salt of sitagliptin is sitagliptin dihydrogen phosphate monohydrate.

102. The pharmaceutical composition of claim 96 wherein sitagliptin is equivalent to 25 mg sitagliptin free base, 50 mg sitagliptin free base, or 100 mg sitagliptin free base.

103. The pharmaceutical composition of claim 96 further comprising a film coating.
